(19) United States
(12) Patent Application Publication
(10) Pub. No.: US 2022/0184222 A1
(43) Pub. Date: Jun. 16, 2022

(54) BICYCLE TOXIN CONJUGATES AND USES THEREOF

(71) Applicant: BicycleTx Limited, Cambridge (GB)

(72) Inventors: Gavin BENNETT, Cambridge (GB); Johanna LAHDENRANTA, Lexington, MA (US)

(21) Appl. No.: 17/594,031

(22) PCT Filed: Apr. 2, 2020
(86) PCT No.: PCT/GB2020/050874

§ 371 Date: Sep. 30, 2021

Related U.S. Application Data
(60) Provisional application No. 62/893,835, filed on Aug. 30, 2019.
(60) Provisional application No. 62/845,954, filed on May 10, 2019.
(60) Provisional application No. 62/827,899, filed on Apr. 2, 2019.

(51) Int. Cl. A61K 47/64; A61P 35/00; A61K 47/65

(52) U.S. Cl. CPC A61K 47/6415; A61K 47/65; A61P 35/00

(57) ABSTRACT

The present invention relates to Bicycle toxin conjugates, or pharmaceutically acceptable salts thereof, or pharmaceutical compositions thereof, and uses thereof for preventing or treating a disease, disorder, or condition characterised by overexpression of EphA2 in diseased tissue, such as cancer.

### TECHNICAL FIELD OF THE INVENTION

The present invention relates to Bicycle toxin conjugates, or pharmaceutically acceptable salts thereof, or pharmaceutical compositions thereof. The present invention also provides uses of Bicycle toxin conjugates, or pharmaceutically acceptable salts thereof, or pharmaceutical compositions thereof, for preventing or treating a disease, disorder, or condition characterised by overexpression of EphA2 in diseased tissue.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al. (2007), Science 330, 1066-71), a cyclic peptide with the Arg-Gly-Asp motif binding to integrin αVb3 (355 Å²) (Xiong et al. (2002), Science 296 (5565), 151-5) or the cyclic peptide inhibitor upain-1 binding to urokinase-type plasminogen activator (603 Å²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8, (NIP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example TATA (1,1′,1″-(1,3,5-triazinane-1,3,5-triyl)triprop-2-en-1-one, Heinis et al. Angew Chem, Int Ed. 2014; 53:1602-1606).

Phage display-based combinatorial approaches have been developed to generate and screen large libraries of bicyclic peptides to targets of interest (Heinis et al. (2009), Nat Chem Biol 5 (7), 502-7 and WO 2009/098450). Briefly, combinatorial libraries of linear peptides containing three cysteine residues and two regions of six random amino acids (Cys-(Xaa)₆-Cys-(Xaa)₆-Cys) (SEQ ID NO: 2) were displayed on phage and cyclised by covalently linking the cysteine side chains to a small molecule scaffold.

### SUMMARY OF THE INVENTION

It had been found that Bicycle toxin conjugates BT5528 and BCY10188, and pharmaceutically acceptable salts and pharmaceutical compositions thereof, are effective in treating diseases, disorders, or conditions characterised by overexpression of EphA2, for example, cancers.

In one aspect, the present invention provides Bicycle toxin conjugate BT5528, or a pharmaceutically acceptable salt thereof, or a pharmaceutical composition thereof.

In one aspect, the present invention provides Bicycle toxin conjugate BCY10188, or a pharmaceutically acceptable salt thereof, or a pharmaceutical composition thereof.

In one aspect, the present invention provides a method of preventing or treating a disease, disorder, or condition characterised by overexpression of EphA2 in a patient, comprising administering to the patient BT5528, or a pharmaceutically acceptable salt thereof, or a pharmaceutical composition thereof.

In one aspect, the present invention provides a method of preventing or treating a disease, disorder, or condition characterised by overexpression of EphA2 in a patient, comprising administering to the patient BCY10188, or a pharmaceutically acceptable salt thereof, or a pharmaceutical composition thereof.

### BRIEF DESCRIPTION OF THE FIGURES

FIG. 1 depicts body weight changes and tumor volume trace after administering BT5528 and BCY10188 to female BALB/c nude mice bearing PC-3 xenograft. Data points represent group mean body weight and tumor volume. Error bars represent standard error of the mean (SEM).

FIG. 2 depicts body weight changes and tumor volume traces after administering BT5528, EphA2-ADC or Docetaxel to male Balb/c nude mice bearing PC-3 xenograft. Data points represent group mean body weight. Error bars represent standard error of the mean (SEM).

FIG. 3 depicts body weight changes and tumor volume traces after administering non-binding BTC or Docetaxel to male Balb/c nude mice bearing PC-3 xenograft. Data points represent group mean body weight. Error bars represent standard error of the mean (SEM).

FIG. 4 depicts (A) concentrations of tumor MMAE, plasma MMAE, and plasma BT5528; and (B) tumor pHH3, after a single dose of BT5528.

FIG. 5 depicts tumor volume traces after treatment in (A) PDX Panc033 xenograft; and (B) PDX Panc163 xenograft. Error bars represent standard error of the mean (SEM).

FIG. 6 depicts: (A) total bone signal; (B) BW change (%); and (C) percentage survival in metastatic PC3 xenograft model after the vehicle and BT5528 treatment.

FIG. 7 depicts tumor volume in models (A) CTG-0160; (B) CTG-0170; (C) CTG-0178; (D) CTG-0192; (E) CTG-0363; (F) CTG-0808; (G) CTG-0838; (H) CTG-0848; (I) CTG-1212; (J) CTG-1502; (K) CTG-1535; (L) CTG-2011; (M) CTG-2393; (N) CTG-2539; and (O) CTG-2540 with weekly dosing of 3 mg/kg BT5528.

FIG. 8 depicts tumor growth inhibition in 15 low-passage Champions TumorGraft® models with weekly dosing of 3 mg/kg BT5528.

### DETAILED DESCRIPTION OF CERTAIN EMBODIMENTS

### 1. General Description of Certain Embodiments of the Invention

It has been found that the compounds of the invention have a number of advantages in preventing and treating EphA2-overexpressing diseases, disorders, and conditions. BT5528 has been found to have a short systemic exposure, to lead to accumulation of MMAE in tumor tissue and mitotic arrest of tumor cells (24-48 h post-dose), and to result in measurable tumor regression by day 4 after dosing.

For example, a single dose of BT5528 is shown to produce high MMAE concentrations in tumour, which is stable from 2 h to >48 h, and to result a transient exposure of both BT5528 and MMAE in plasma. A single dose of BT5528 is also shown to induce mitotic arrest in tumor, which is measurable by pHH3 IHC within 24 hours. BT5528 is also found to show equivalent efficacy with a wide range of dosing paradigms, for example, via iv bolus (QW×2 and QW×4), 1 h iv infusion (QW×2), or via 24 h delivery from subcutaneously implanted osmotic pump (QW×2). BT5528 is also found to be efficacious with intermittent dosing, for example, dosing every 2 weeks.

Without wishing to be bound by any specific theory, BT5528 activity is likely a combination of targeted internalization and bystander effect of MMAE. It has been found that target-mediated internalization of poorly membrane permeable payload MMAF leads to suboptimal anti-tumor activity as compared to BT5528. For example, 1 mpk BT5528 leads to robust tumor regressions (TGI 111%), and 1 mpk BCY10188 slows down tumor growth (TGI 80%). Part of the EphA2-MMAE BTC (BT5528) activity in vivo is likely due to target-dependent internalization of the payload. EphA2-MMAF BTC (BCY10188) gets actively internalized into tumor cells. Bystander effect (i.e. release of the payload in the protease rich tumor microenvironment and diffusion into tumor cells) is required for maximum anti-tumor activity of BT5528.

In some embodiments, the present invention provides Bicycle toxin conjugate BT5528, or a pharmaceutically acceptable salt thereof.

In some embodiments, the present invention provides a method of preventing or treating a disease, disorder, or condition characterised by overexpression of EphA2 in a patient, comprising administering to the patient Bicycle toxin conjugate BT5528, or a pharmaceutically acceptable salt thereof, or a pharmaceutical composition thereof.

In some embodiments, the present invention provides Bicycle toxin conjugate BCY10188, or a pharmaceutically acceptable salt thereof.

In some embodiments, the present invention provides a method of preventing or treating a disease, disorder, or condition characterised by overexpression of EphA2 in a patient, comprising administering to the patient Bicycle toxin conjugate BCY10188, or a pharmaceutically acceptable salt thereof, or a pharmaceutical composition thereof.

### 2. Compounds and Definitions

The term “BT5528,” as used herein, is a Bicycle toxin conjugate having a structure as shown below, wherein the molecular scaffold is 1,1′,1″-(1,3,5-triazinane-1,3,5-triyl)triprop-2-en-1-one (TATA), and the peptide ligand comprises the amino acid sequence:

(β-Ala)-Sar₁₀-A(HArg)D-Cᵢ(HyP)LVNPLCᵢᵢLHP(D-Asp)W(HArg)Cᵢᵢᵢ (SEQ ID NO: 1) wherein Sar is sarcosine, HArg is homoarginine, and HyP is hydroxyproline.

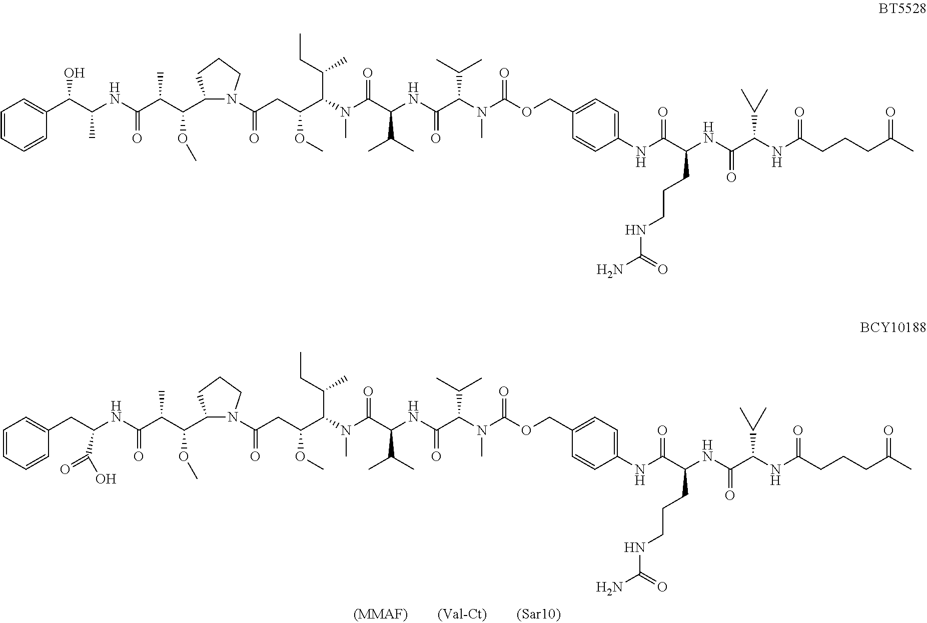

The term “BCY10188,” as used herein, is a Bicycle toxin conjugate having a structure as shown above, wherein the molecular scaffold is 1,1′,1″-(1,3,5-triazinane-1,3,5-triyl)triprop-2-en-1-one (TATA), and the peptide ligand comprises the amino acid sequence:

(β-Ala)-Sar₁₀-A(HArg)D-Cᵢ(HyP)LVNPLCᵢᵢLHP(D-Asp)W(HArg)Cᵢᵢᵢ(SEQ ID NO: 1) wherein Sar is sarcosine, HArg is homoarginine, and HyP is hydroxyproline. BCY10188 and BT5528 only differ in that the toxin moiety in BCY10188 is MMAF, while the toxin moiety in BT5528 is MMAE.

As used herein, the term “Monomethyl auristatin E” or “MMAE” refers to a compound of the following structure:

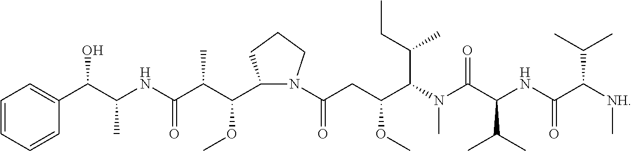 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

As used herein, the term “Monomethyl auristatin F” or “MMAF” refers to a compound of the following structure:

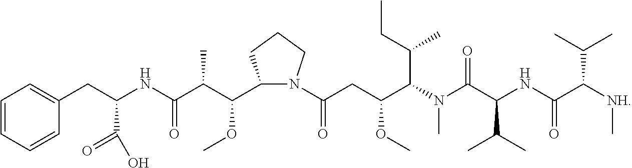 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

As used herein, the term “about” shall have the meaning of within 10% of a given value or range. In some embodiments, the term “about” refers to within 10%, 9%, 8%, 7%, 6%, 5%, 4%, 3%, 2%, or 1% of a given value.

As used herein, the term “mg/kg” refers to the milligram of medication per kilogram of the body weight of the subject taking the medication. As provided by the FDA guidance, a dose in mg/kg in an animal can be converted to a corresponding Human Equivalent Dose (HED) in mg/m². For example, a conversion between doses in mouse and HED is shown below:

| Mouse Dose | Human Equivalent |
|---|---|
| (mg/kg) | Dose (mg/m²) |
| 0.05 | 0.15 |
| 0.1 | 0.3 |
| 0.11 | 0.33 |
| 0.2 | 0.6 |
| 0.3 | 0.9 |
| 0.33 | 0.99 |
| 0.5 | 1.5 |
| 1.0 | 3.0 |
| 1.5 | 4.5 |
| 2.0 | 6.0 |
| 2.5 | 7.5 |
| 3.0 | 9.0 |
| 5.0 | 15.0 |
| 10.0 | 30.0 |
| 15.0 | 45.0 |

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

### 3. Description of Exemplary Embodiments

In some embodiments, the present invention provides Bicycle toxin conjugate BT5528, or a pharmaceutically acceptable salt thereof.

In some embodiments, the present invention provides a pharmaceutical composition comprising Bicycle toxin conjugate BT5528, or a pharmaceutically acceptable salt thereof, and a pharmaceutically acceptable carrier, adjuvant, or vehicle.

In some embodiments, the present invention provides a method of preventing or treating a disease, disorder, or condition characterised by overexpression of EphA2 in a patient, comprising administering to the patient Bicycle toxin conjugate BT5528, or a pharmaceutically acceptable salt thereof, or a pharmaceutical composition thereof.

In some embodiments, Bicycle toxin conjugate BT5528, or a pharmaceutically acceptable salt thereof, or a pharmaceutical composition thereof, is administered to a patient to provide a system exposure of BT5528 and/or MMAE for about 4 hours or less. In some embodiments, Bicycle toxin conjugate BT5528, or a pharmaceutically acceptable salt thereof, or a pharmaceutical composition thereof, is administered to a patient to provide a system exposure of BT5528 and/or MMAE for about 0.5-4, or 0.5-3, or 0.5-2, or 1-3, or 1-2 hours. In some embodiments, Bicycle toxin conjugate BT5528, or a pharmaceutically acceptable salt thereof, or a pharmaceutical composition thereof, is administered to a patient to provide a system exposure of BT5528 and/or MMAE for about 3.5, or 3.0, or 2.5, or 2.0, or 1.5, or 1.0, or 0.5 hours. In some embodiments, a system exposure of BT5528 and/or MMAE as described herein is achieved by an administration of BT5528, or a pharmaceutically acceptable salt thereof, or a pharmaceutical composition thereof, at a dosage level as described herein. In some embodiments, a system exposure of BT5528 and/or MMAE as described herein is achieved by an administration of BT5528, or a pharmaceutically acceptable salt thereof, or a pharmaceutical composition thereof, at a dosing interval as described herein. In some embodiments, a system exposure of BT5528 and/or MMAE as described herein is achieved by an administration of BT5528, or a pharmaceutically acceptable salt thereof, or a pharmaceutical composition thereof, via a route as described herein.

In some embodiments, a system exposure of BT5528 is measured by the time when the concentration of BT5528 in plasma is about 20% or more, or about 18% or more, or about 16% or more, or about 14% or more, or about 12% or more, or about 10% or more, of the maximum concentration of BT5528 in plasma. In some embodiments, a system exposure of BT5528 is measured by the time when the concentration of BT5528 in plasma is about 15% or more of the maximum concentration of BT5528 in plasma. In some embodiments, a system exposure of BT5528 is measured by the time when the concentration of BT5528 in plasma is about 8% or more, or about 6% or more, or about 4% or more, or about 2% or more, of the maximum concentration of BT5528 in plasma. In some embodiments, a system exposure of BT5528 is measured by the time when the concentration of BT5528 in plasma is about 5% or more of the maximum concentration of BT5528 in plasma. In some embodiments, a system exposure of BT5528 is measured by the time when the concentration of BT5528 in plasma is about 10 pmol/gram or more, or about 12 pmol/gram or more, or about 14 pmol/gram or more, or about 16 pmol/gram or more, or about 18 pmol/gram or more, or about 20 pmol/gram or more. In some embodiments, a system exposure of BT5528 is measured by the time when the concentration of BT5528 in plasma is about 15 pmol/gram or more. In some embodiments, a system exposure of BT5528 is measured by the time when the concentration of BT5528 in plasma is about 22 pmol/gram or more, or about 24 pmol/gram or more, or about 26 pmol/gram or more, or about 28 pmol/gram or more, or about 30 pmol/gram or more. In some embodiments, a system exposure of BT5528 is measured by the time when the concentration of BT5528 in plasma is about 32 pmol/gram or more, or about 34 pmol/gram or more, or about 36 pmol/gram or more, or about 38 pmol/gram or more, or about 40 pmol/gram or more. In some embodiments, a system exposure of BT5528 is measured by the time when the concentration of BT5528 in plasma is about 40-50 pmol/gram or more.

In some embodiments, a system exposure of MMAE is measured by the time when the concentration of MMAE in plasma is about 20% or more, or about 18% or more, or about 16% or more, or about 14% or more, or about 12% or more, of the maximum concentration of MMAE in plasma. In some embodiments, a system exposure of MMAE is measured by the time when the concentration of MMAE in plasma is about 10% or more of the maximum concentration of MMAE in plasma. In some embodiments, a system exposure of MMAE is measured by the time when the concentration of MMAE in plasma is about 8% or more, or about 6% or more, or about 4% or more, or about 2% or more, of the maximum concentration of MMAE in plasma. In some embodiments, a system exposure of MMAE is measured by the time when the concentration of MMAE in plasma is about 1 pmol/gram or more, or about 1.2 pmol/gram or more, or about 1.4 pmol/gram or more, or about 1.6 pmol/gram or more, or about 1.8 pmol/gram or more. In some embodiments, a system exposure of MMAE is measured by the time when the concentration of MMAE in plasma is about 2 pmol/gram or more. In some embodiments, a system exposure of MMAE is measured by the time when the concentration of MMAE in plasma is about 2.2 pmol/gram or more, or about 2.4 pmol/gram or more, or about 2.6 pmol/gram or more, or about 2.8 pmol/gram or more. In some embodiments, a system exposure of MMAE is measured by the time when the concentration of MMAE in plasma is about 3 pmol/gram or more.

In some embodiments, Bicycle toxin conjugate BT5528, or a pharmaceutically acceptable salt thereof, or a pharmaceutical composition thereof, is administered to a patient to provide a tumor MMAE concentration of about 20 pmol/gram or more, or about 22 pmol/gram or more, or about 24 pmol/gram or more, or about 26 pmol/gram or more, or about 28 pmol/gram or more. In some embodiments, Bicycle toxin conjugate BT5528, or a pharmaceutically acceptable salt thereof, or a pharmaceutical composition thereof, is administered to a patient to provide a tumor MMAE concentration of about 30 pmol/gram or more, or about 32 pmol/gram or more, or about 34 pmol/gram or more, or about 36 pmol/gram or more, or about 38 pmol/gram or more. In some embodiments, Bicycle toxin conjugate BT5528, or a pharmaceutically acceptable salt thereof, or a pharmaceutical composition thereof, is administered to a patient to provide a tumor MMAE concentration of about 40 pmol/gram or more, or about 42 pmol/gram or more, or about 44 pmol/gram or more, or about 46 pmol/gram or more, or about 48 pmol/gram or more. In some embodiments, Bicycle toxin conjugate BT5528, or a pharmaceutically acceptable salt thereof, or a pharmaceutical composition thereof, is administered to a patient to provide a tumor MMAE concentration of about 50 pmol/gram or more. In some embodiments, Bicycle toxin conjugate BT5528, or a pharmaceutically acceptable salt thereof, or a pharmaceutical composition thereof, is administered to a patient to provide a tumor MMAE concentration of about 55 pmol/gram or more, or about 60 pmol/gram or more. In some embodiments, a tumor MMAE concentration as described herein is achieved by an administration of BT5528, or a pharmaceutically acceptable salt thereof, or a pharmaceutical composition thereof, at a dosage level as described herein. In some embodiments, a tumor MMAE concentration as described herein is achieved by an administration of BT5528, or a pharmaceutically acceptable salt thereof, or a pharmaceutical composition thereof, at a dosing interval as described herein. In some embodiments, a tumor MMAE concentration as described herein is achieved by an administration of BT5528, or a pharmaceutically acceptable salt thereof, or a pharmaceutical composition thereof, via a route as described herein.

In some embodiments, Bicycle toxin conjugate BT5528, or a pharmaceutically acceptable salt thereof, or a pharmaceutical composition thereof, is administered to a patient to induce mitotic arrest in tumor within about 12-48 hours. In some embodiments, Bicycle toxin conjugate BT5528, or a pharmaceutically acceptable salt thereof, or a pharmaceutical composition thereof, is administered to a patient to induce mitotic arrest in tumor within about 12-18 hours, or about 18-24 hours, or about 24-30 hours, or about 30-36 hours, or about 36-42 hours, or about 42-48 hours. In some embodiments, Bicycle toxin conjugate BT5528, or a pharmaceutically acceptable salt thereof, or a pharmaceutical composition thereof, is administered to a patient to induce mitotic arrest in tumor within about 16, 18, 20, 22, 24, 26, 28, 30, or 32 hours. In some embodiments, an induction of mitotic arrest in tumor as described herein is achieved by an administration of BT5528, or a pharmaceutically acceptable salt thereof, or a pharmaceutical composition thereof, at a dosage level as described herein. In some embodiments, an induction of mitotic arrest in tumor as described herein is achieved by an administration of BT5528, or a pharmaceutically acceptable salt thereof, or a pharmaceutical composition thereof, at a dosing interval as described herein. In some embodiments, an induction of mitotic arrest in tumor as described herein is achieved by an administration of BT5528, or a pharmaceutically acceptable salt thereof, or a pharmaceutical composition thereof, via a route as described herein.

In some embodiments, mitotic arrest in tumor is induced when there is about 4% or more, or about 6% or more, or about 8% or more, or about 10% or more, pHH3+ nuclei in tumor. In some embodiments, mitotic arrest in tumor is induced when there is about 12% or more, or about 14% or more, or about 16% or more, or about 18% or more, or about 20% or more, pHH3+ nuclei in tumor. In some embodiments, mitotic arrest in tumor is induced when there is about 15% pHH3+ nuclei or more in tumor.

In some embodiments, Bicycle toxin conjugate BT5528, or a pharmaceutically acceptable salt thereof, or a pharmaceutical composition thereof, is administered to a patient to induce measurable tumor regression by day 7 post dosing. In some embodiments, Bicycle toxin conjugate BT5528, or a pharmaceutically acceptable salt thereof, or a pharmaceutical composition thereof, is administered to a patient to induce measurable tumor regression by day 6 post dosing. In some embodiments, Bicycle toxin conjugate BT5528, or a pharmaceutically acceptable salt thereof, or a pharmaceutical composition thereof, is administered to a patient to induce measurable tumor regression by day 5 post dosing. In some embodiments, Bicycle toxin conjugate BT5528, or a pharmaceutically acceptable salt thereof, or a pharmaceutical composition thereof, is administered to a patient to induce measurable tumor regression by day 4 post dosing. In some embodiments, Bicycle toxin conjugate BT5528, or a pharmaceutically acceptable salt thereof, or a pharmaceutical composition thereof, is administered to a patient to induce measurable tumor regression by day 3 post dosing. In some embodiments, Bicycle toxin conjugate BT5528, or a pharmaceutically acceptable salt thereof, or a pharmaceutical composition thereof, is administered to a patient to induce measurable tumor regression by day 2 post dosing. In some embodiments, an induction of measurable tumor regression as described herein is achieved by an administration of BT5528, or a pharmaceutically acceptable salt thereof, or a pharmaceutical composition thereof, at a dosage level as described herein. In some embodiments, an induction of measurable tumor regression as described herein is achieved by an administration of BT5528, or a pharmaceutically acceptable salt thereof, or a pharmaceutical composition thereof, at a dosing interval as described herein. In some embodiments, an induction of measurable tumor regression as described herein is achieved by an administration of BT5528, or a pharmaceutically acceptable salt thereof, or a pharmaceutical composition thereof, via a route as described herein.

In some embodiments, Bicycle toxin conjugate BT5528, or a pharmaceutically acceptable salt thereof, or a pharmaceutical composition thereof, is administered to a patient at about 0.1 mg/kg to about 3 mg/kg each dose. In some embodiments, Bicycle toxin conjugate BT5528, or a pharmaceutically acceptable salt thereof, or a pharmaceutical composition thereof, is administered to a patient at about 0.11 mg/kg each dose. In some embodiments, Bicycle toxin conjugate BT5528, or a pharmaceutically acceptable salt thereof, or a pharmaceutical composition thereof, is administered to a patient at about 0.33 mg/kg each dose. In some embodiments, Bicycle toxin conjugate BT5528, or a pharmaceutically acceptable salt thereof, or a pharmaceutical composition thereof, is administered to a patient at about 0.5 mg/kg each dose. In some embodiments, Bicycle toxin conjugate BT5528, or a pharmaceutically acceptable salt thereof, or a pharmaceutical composition thereof, is administered to a patient at about 1.0 mg/kg each dose. In some embodiments, Bicycle toxin conjugate BT5528, or a pharmaceutically acceptable salt thereof, or a pharmaceutical composition thereof, is administered to a patient at about 3 mg/kg each dose.

In some embodiments, Bicycle toxin conjugate BT5528, or a pharmaceutically acceptable salt thereof, or a pharmaceutical composition thereof, is administered to a patient at about 0.3 mg/m² to about 9 mg/m² each dose. In some embodiments, Bicycle toxin conjugate BT5528, or a pharmaceutically acceptable salt thereof, or a pharmaceutical composition thereof, is administered to a patient at about 0.33 mg/m² each dose. In some embodiments, Bicycle toxin conjugate BT5528, or a pharmaceutically acceptable salt thereof, or a pharmaceutical composition thereof, is administered to a patient at about 0.99 mg/m² each dose. In some embodiments, Bicycle toxin conjugate BT5528, or a pharmaceutically acceptable salt thereof, or a pharmaceutical composition thereof, is administered to a patient at about 1.5 mg/m² each dose. In some embodiments, Bicycle toxin conjugate BT5528, or a pharmaceutically acceptable salt thereof, or a pharmaceutical composition thereof, is administered to a patient at about 3.0 mg/m² each dose. In some embodiments, Bicycle toxin conjugate BT5528, or a pharmaceutically acceptable salt thereof, or a pharmaceutical composition thereof, is administered to a patient at about 9 mg/m² each dose.

In some embodiments, Bicycle toxin conjugate BT5528, or a pharmaceutically acceptable salt thereof, or a pharmaceutical composition thereof, is administered to a patient by an intravenous bolus injection.

In some embodiments, Bicycle toxin conjugate BT5528, or a pharmaceutically acceptable salt thereof, or a pharmaceutical composition thereof, is administered to a patient by an intravenous infusion. In some embodiments, an intravenous infusion of Bicycle toxin conjugate BT5528, or a pharmaceutically acceptable salt thereof, or a pharmaceutical composition thereof, is a 5-10 minute infusion. In some embodiments, an intravenous infusion of Bicycle toxin conjugate BT5528, or a pharmaceutically acceptable salt thereof, or a pharmaceutical composition thereof, is a 10-20 minute infusion. In some embodiments, an intravenous infusion of Bicycle toxin conjugate BT5528, or a pharmaceutically acceptable salt thereof, or a pharmaceutical composition thereof, is a 20-40 minute infusion. In some embodiments, an intravenous infusion of Bicycle toxin conjugate BT5528, or a pharmaceutically acceptable salt thereof, or a pharmaceutical composition thereof, is an about 45, or 50, or 55 minute infusion. In some embodiments, an intravenous infusion of Bicycle toxin conjugate BT5528, or a pharmaceutically acceptable salt thereof, or a pharmaceutical composition thereof, is an about 1 hour infusion. In some embodiments, an intravenous infusion of Bicycle toxin conjugate BT5528, or a pharmaceutically acceptable salt thereof, or a pharmaceutical composition thereof, is a 1-1.5 hr infusion. In some embodiments, an intravenous infusion of Bicycle toxin conjugate BT5528, or a pharmaceutically acceptable salt thereof, or a pharmaceutical composition thereof, is a 1.5-2 hr infusion. In some embodiments, an intravenous infusion of Bicycle toxin conjugate BT5528, or a pharmaceutically acceptable salt thereof, or a pharmaceutical composition thereof, is a 2-3 hr infusion. In some embodiments, an intravenous infusion of Bicycle toxin conjugate BT5528, or a pharmaceutically acceptable salt thereof, or a pharmaceutical composition thereof, is a more than 3 hr infusion.

In some embodiments, Bicycle toxin conjugate BT5528, or a pharmaceutically acceptable salt thereof, or a pharmaceutical composition thereof, is administered to a patient by a subcutaneous infusion. In some embodiments, a subcutaneous infusion of Bicycle toxin conjugate BT5528, or a pharmaceutically acceptable salt thereof, or a pharmaceutical composition thereof, is an about 1-5 hr infusion. In some embodiments, a subcutaneous infusion of Bicycle toxin conjugate BT5528, or a pharmaceutically acceptable salt thereof, or a pharmaceutical composition thereof, is an about 5-10 hr infusion. In some embodiments, a subcutaneous infusion of Bicycle toxin conjugate BT5528, or a pharmaceutically acceptable salt thereof, or a pharmaceutical composition thereof, is an about 10-15 hr infusion. In some embodiments, a subcutaneous infusion of Bicycle toxin conjugate BT5528, or a pharmaceutically acceptable salt thereof, or a pharmaceutical composition thereof, is an about 15-20 hr infusion. In some embodiments, a subcutaneous infusion of Bicycle toxin conjugate BT5528, or a pharmaceutically acceptable salt thereof, or a pharmaceutical composition thereof, is an about 20, or 21, or 22, or 24 hr infusion. In some embodiments, a subcutaneous infusion of Bicycle toxin conjugate BT5528, or a pharmaceutically acceptable salt thereof, or a pharmaceutical composition thereof, is an about 24 hr infusion. In some embodiments, a subcutaneous infusion of Bicycle toxin conjugate BT5528, or a pharmaceutically acceptable salt thereof, or a pharmaceutical composition thereof, is a 1-1.5 day infusion. In some embodiments, a subcutaneous infusion of Bicycle toxin conjugate BT5528, or a pharmaceutically acceptable salt thereof, or a pharmaceutical composition thereof, is a 1.5-2 day infusion. In some embodiments, a subcutaneous infusion of Bicycle toxin conjugate BT5528, or a pharmaceutically acceptable salt thereof, or a pharmaceutical composition thereof, is a 2-5 day infusion. In some embodiments, a subcutaneous infusion of Bicycle toxin conjugate BT5528, or a pharmaceutically acceptable salt thereof, or a pharmaceutical composition thereof, is a more than 5 day infusion.

In some embodiments, Bicycle toxin conjugate BT5528, or a pharmaceutically acceptable salt thereof, or a pharmaceutical composition thereof, is administered to a patient two or more times, with at least 24 hours in between two consecutive administrations. In some embodiments, Bicycle toxin conjugate BT5528, or a pharmaceutically acceptable salt thereof, or a pharmaceutical composition thereof, is administered to a patient two or more times, with 24-48 hours in between two consecutive administrations. In some embodiments, Bicycle toxin conjugate BT5528, or a pharmaceutically acceptable salt thereof, or a pharmaceutical composition thereof, is administered to a patient two or more times, with about 3, or 4, or 5, or 6 days in between two consecutive administrations. In some embodiments, Bicycle toxin conjugate BT5528, or a pharmaceutically acceptable salt thereof, or a pharmaceutical composition thereof, is administered to a patient two or more times, with about one week in between two consecutive administrations. In some embodiments, Bicycle toxin conjugate BT5528, or a pharmaceutically acceptable salt thereof, or a pharmaceutical composition thereof, is administered to a patient two or more times, with about 1.5 or 2 weeks in between two consecutive administrations. In some embodiments, Bicycle toxin conjugate BT5528, or a pharmaceutically acceptable salt thereof, or a pharmaceutical composition thereof, is administered to a patient two or more times, with about three weeks in between two consecutive administrations. In some embodiments, Bicycle toxin conjugate BT5528, or a pharmaceutically acceptable salt thereof, or a pharmaceutical composition thereof, is administered to a patient two or more times, with about four weeks in between two consecutive administrations.

In some embodiments, the present invention provides Bicycle toxin conjugate BCY10188, or a pharmaceutically acceptable salt thereof.

In some embodiments, the present invention provides a pharmaceutical composition comprising Bicycle toxin conjugate BCY10188, or a pharmaceutically acceptable salt thereof, and a pharmaceutically acceptable carrier, adjuvant, or vehicle.

In some embodiments, the present invention provides a method of preventing or treating a disease, disorder, or condition characterised by overexpression of EphA2 in a patient, comprising administering to the patient Bicycle toxin conjugate BCY10188, or a pharmaceutically acceptable salt thereof, or a pharmaceutical composition thereof.

In some embodiments, Bicycle toxin conjugate BCY10188, or a pharmaceutically acceptable salt thereof, or a pharmaceutical composition thereof is administered at about 0.05-15 mg/kg each dose. In some embodiments, Bicycle toxin conjugate BCY10188, or a pharmaceutically acceptable salt thereof, or a pharmaceutical composition thereof is administered at about 0.1-10 mg/kg each dose. In some embodiments, Bicycle toxin conjugate BCY10188, or a pharmaceutically acceptable salt thereof, or a pharmaceutical composition thereof is administered at about 0.2-5 mg/kg each dose. In some embodiments, Bicycle toxin conjugate BCY10188, or a pharmaceutically acceptable salt thereof, or a pharmaceutical composition thereof is administered at about 0.3-3 mg/kg each dose. In some embodiments, Bicycle toxin conjugate BCY10188, or a pharmaceutically acceptable salt thereof, or a pharmaceutical composition thereof is administered at about 0.5-3 mg/kg each dose. In some embodiments, Bicycle toxin conjugate BCY10188, or a pharmaceutically acceptable salt thereof, or a pharmaceutical composition thereof is administered at about 0.5, or 1.0, or 1.5, or 2.0, or 2.5, or 3.0 mg/kg each dose.

In some embodiments, Bicycle toxin conjugate BCY10188, or a pharmaceutically acceptable salt thereof, or a pharmaceutical composition thereof is administered at about 0.15-45 mg/m² each dose. In some embodiments, Bicycle toxin conjugate BCY10188, or a pharmaceutically acceptable salt thereof, or a pharmaceutical composition thereof is administered at about 0.3-30 mg/m² each dose. In some embodiments, Bicycle toxin conjugate BCY10188, or a pharmaceutically acceptable salt thereof, or a pharmaceutical composition thereof is administered at about 0.6-15 mg/m² each dose. In some embodiments, Bicycle toxin conjugate BCY10188, or a pharmaceutically acceptable salt thereof, or a pharmaceutical composition thereof is administered at about 0.9-9 mg/m² each dose. In some embodiments, Bicycle toxin conjugate BCY10188, or a pharmaceutically acceptable salt thereof, or a pharmaceutical composition thereof is administered at about 1.5-9 mg/m² each dose. In some embodiments, Bicycle toxin conjugate BCY10188, or a pharmaceutically acceptable salt thereof, or a pharmaceutical composition thereof is administered at about 1.5, or 3.0, or 4.5, or 6.0, or 7.5, or 9.0 mg/m² each dose.

In some embodiments, Bicycle toxin conjugate BT10188, or a pharmaceutically acceptable salt thereof, or a pharmaceutical composition thereof, is administered to a patient by an intravenous bolus injection.

In some embodiments, Bicycle toxin conjugate BT10188, or a pharmaceutically acceptable salt thereof, or a pharmaceutical composition thereof, is administered to a patient by an intravenous infusion. In some embodiments, Bicycle toxin conjugate BT10188, or a pharmaceutically acceptable salt thereof, or a pharmaceutical composition thereof, is administered to a patient by an intravenous infusion as described herein for BT5528.

In some embodiments, Bicycle toxin conjugate BT10118, or a pharmaceutically acceptable salt thereof, or a pharmaceutical composition thereof, is administered to a patient by a subcutaneous infusion. In some embodiments, Bicycle toxin conjugate BT10188, or a pharmaceutically acceptable salt thereof, or a pharmaceutical composition thereof, is administered to a patient by a subcutaneous infusion as described herein for BT5528.

In some embodiments, Bicycle toxin conjugate BT10188, or a pharmaceutically acceptable salt thereof, or a pharmaceutical composition thereof, is administered to a patient two or more times, with an interval between two consecutive administrations as described herein for BT5528.

In some embodiments, a disease, disorder, or condition characterised by overexpression of EphA2 is a cancer. In some embodiments, a cancer is selected from those as described herein. In some embodiments, a cancer is a pancreatic cancer. In some embodiments, a cancer is metastatic cancer as described herein. In some embodiments, a cancer is a drug-resistant cancer as described herein. In some embodiments, a cancer is prostate cancer. In some embodiments, a cancer is metastatic prostate cancer.

In some embodiments, the present invention provides a method of preventing or treating a disease, disorder, or condition characterised by overexpression of EphA2 in a patient, comprising administering to the patient Bicycle toxin conjugate BT5528 or BCY10188, or a pharmaceutically acceptable salt thereof, or a pharmaceutical composition thereof, in combination with one or more other therapeutic agent as described herein.

### 4. Uses, Formulation and Administration

### Pharmaceutically Acceptable Compositions

According to some embodiments, the invention provides a composition comprising a compound of this invention (BT5528 or BCY10188), or a pharmaceutically acceptable derivative thereof, and a pharmaceutically acceptable carrier, adjuvant, or vehicle.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

According to some embodiments, the present invention provides a method of preventing or treating a disease, disorder, or condition characterised by overexpression of EphA2 in a patient, comprising administering to the patient a compound of this invention (BT5528 or BCY10188), or a pharmaceutically acceptable derivative thereof, or a pharmaceutical composition thereof.

In some embodiments, a disease, disorder, or condition characterised by overexpression of EphA2 is cancer.

### Cancer

Cancer includes, in one embodiment, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, a cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, a cancer is a viral-associated cancer, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/NCT02426892)

In some embodiments, a cancer is melanoma cancer. In some embodiments, a cancer is breast cancer. In some embodiments, a cancer is lung cancer. In some embodiments, a cancer is small cell lung cancer (SCLC). In some embodiments, a cancer is non-small cell lung cancer (NSCLC).

In some embodiments, a cancer is treated by arresting further growth of the tumor. In some embodiments, a cancer is treated by reducing the size (e.g., volume or mass) of the tumor by at least 5%, 10%, 25%, 50%, 75%, 90% or 99% relative to the size of the tumor prior to treatment. In some embodiments, a cancer is treated by reducing the quantity of the tumor in the patient by at least 5%, 10%, 25%, 50%, 75%, 90% or 99% relative to the quantity of the tumor prior to treatment.

The compounds and compositions, according to the method of the present invention, may be administered using any amount and any route of administration effective for treating or lessening the severity of a cancer. The exact amount required will vary from subject to subject, depending on the species, age, and general condition of the subject, the severity of the disease or condition, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the disease or disorder being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### 5. Co-Administration with One or More Other Therapeutic Agent

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours apart.

As used herein, the term “combination,” “combined,” and related terms refers to the simultaneous or sequential administration of therapeutic agents in accordance with this invention. For example, a compound of the present invention may be administered with one or more other therapeutic agent simultaneously or sequentially in separate unit dosage forms or together in a single unit dosage form. Accordingly, the present invention provides a single unit dosage form comprising a compound of the current invention, one or more other therapeutic agent, and a pharmaceutically acceptable carrier, adjuvant, or vehicle.

The amount of a compound of the invention and one or more other therapeutic agent (in those compositions which comprise an additional therapeutic agent as described above) that may be combined with the carrier materials to produce a single dosage form will vary depending upon the host treated and the particular mode of administration. Preferably, a composition of the invention should be formulated so that a dosage of between 0.01-100 mg/kg body weight/day of a compound of the invention can be administered.

In those compositions which comprise one or more other therapeutic agent, the one or more other therapeutic agent and a compound of the invention may act synergistically. Therefore, the amount of the one or more other therapeutic agent in such compositions may be less than that required in a monotherapy utilizing only that therapeutic agent. In such compositions a dosage of between 0.01-1,000 μg/kg body weight/day of the one or more other therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is approvided for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### Exemplary Other Therapeutic Agents

In some embodiments, one or more other therapeutic agent is a Poly ADP ribose polymerase (PARP) inhibitor. In some embodiments, a PARP inhibitor is selected from olaparib (Lynparza®, AstraZeneca); rucaparib (Rubraca®, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (Zolinza®, Merck); romidepsin (Istodax®, Celgene); panobinostat (Farydak®, Novartis); belinostat (Beleodaq®, Spectrum Pharmaceuticals); entinostat (SNDX-275, Syndax Pharmaceuticals) (NCT00866333); and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance®, Pfizer); ribociclib (Kisqali®, Novartis); abemaciclib (Ly2835219, Eli Lilly); and trilaciclib (G1T28, G1 Therapeutics).

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In some embodiments, one or more other therapeutic agent is a platinum-based therapeutic, also referred to as platins. Platins cause cross-linking of DNA, such that they inhibit DNA repair and/or DNA synthesis, mostly in rapidly reproducing cells, such as cancer cells. In some embodiments, a platinum-based therapeutic is selected from cisplatin (Platinol®, Bristol-Myers Squibb); carboplatin (Paraplatin®, Bristol-Myers Squibb; also, Teva; Pfizer); oxaliplatin (Eloxitin® Sanofi-Aventis); nedaplatin (Aqupla®, Shionogi), picoplatin (Poniard Pharmaceuticals); and satraplatin (JM-216, Agennix).

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaecuticals); vatalanib (Bayer/Novartis); lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is a proteasome inhibitor. Approved proteasome inhibitors useful in the present invention include bortezomib (Velcade®, Takeda); carfilzomib (Kyprolis®, Amgen); and ixazomib (Ninlaro®, Takeda).

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

In some embodiments, one or more other therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, one or more other therapeutic agent is a folic acid inhibitor. Approved folic acid inhibitors useful in the present invention include pemetrexed (Alimta®, Eli Lilly).

In some embodiments, one or more other therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (Xtandi®, Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFβ). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGFβ trap compound (NCT02699515); and (NCT02517398). M7824 is comprised of a fully human IgG1 antibody against PD-L1 fused to the extracellular domain of human TGF-beta receptor II, which functions as a TGFβ “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™ Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™) daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™ Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; Zd₆474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code numbers, generic or trade names may be taken from the actual edition of the standard compendium “The Merck Index” or from databases, e.g. Patents International (e.g. IMS World Publications).

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MEDI4736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+ (Th17) and CD8+ (Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams ET. AL., “Big opportunities for small molecules in immuno-oncology,” Cancer Therapy 2015, Vol. 14, pages 603-622, the content of which is incorporated herein by reference in its entirety. In some embodiment, an immuno-oncology agent is selected from the examples described in Table 1 of Jerry L. Adams ET. AL. In some embodiments, an immuno-oncology agent is a small molecule targeting an immuno-oncology target selected from those listed in Table 2 of Jerry L. Adams ET. AL. In some embodiments, an immuno-oncology agent is a small molecule agent selected from those listed in Table 2 of Jerry L. Adams ET. AL.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negatgive cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (up) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NK(G2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

The following examples are intended to illustrate the invention and are not to be construed as being limitations thereon. All amino acids, unless noted otherwise, were used in the L-configurations.

| Abbre- | | | Precursor | |
|---|---|---|---|---|
| viations | Name | Precursor Name | CAS | Supplier |
| Ac | Acetyl | | | |
| β-Ala | β-Alanine | Fmoc-β-alanine | 35737- | Fluorochem |
| | | | 10-1 | |
| D-Asp | D-Aspartic acid | Fmoc-D-aspartic acid | 112883- | Sigma |
| | | 4-tert-butyl ester | 39-3 | aldrich |
| HArg | HomoArginine | Fmoc-L- | 401915- | Fluorochem |
| | | HomoArg(Pbf)-OH | 53-5 | |
| HyP | Hydroxyproline | Fmoc- | 122996- | Sigma |
| | | Hydroxyproline(tBu)- | 47-8 | |
| | | OH | | |
| Sar | Sarcosine, such | Fmoc-Sarcosine-OH | 77128- | Sigma |
| | that Sarₓ | | 70-2 | |
| | represents x Sar | | | |
| | residues | | | |

### Example 1: Synthesis of BT5528 and BCY10188

### Preparation of Bicycle Peptide 1

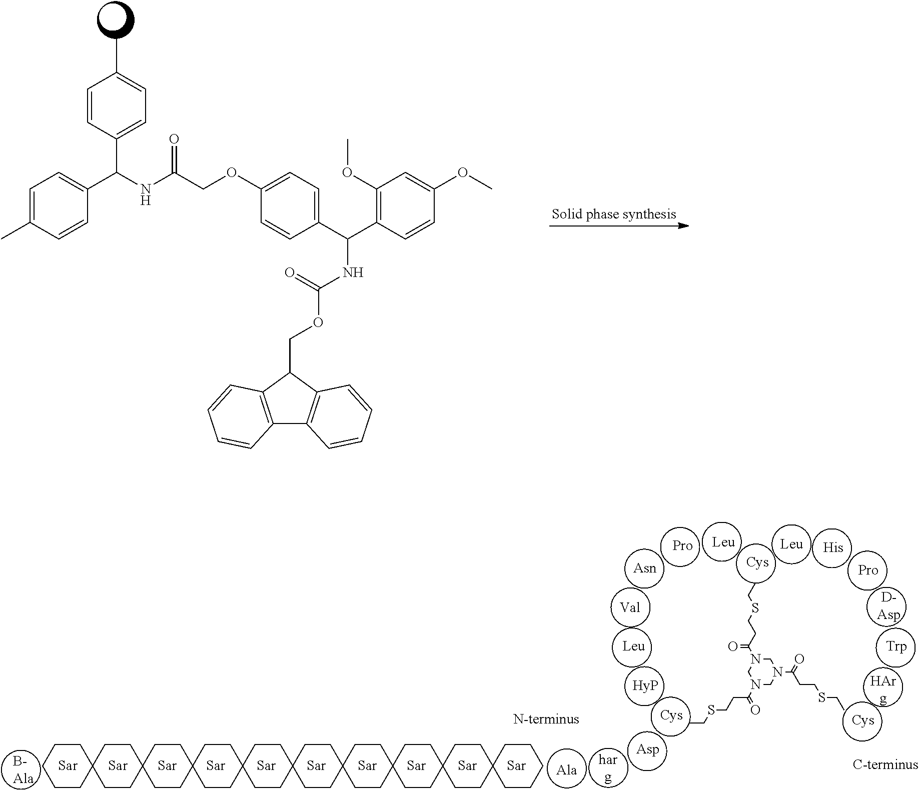

Peptides were synthesized by solid phase synthesis. Rink Amide MBHA Resin was used. To a mixture containing Rink Amide MBHA (0.4-0.45 mmol/g) and Fmoc-Cys(Trt)-OH (3.0 eq) was added DMF, then DIC (3 eq) and HOAt (3 eq) were added and mixed for 1 hour. 20% piperidine in DMF was used for deblocking. Each subsequent amino acid was coupled with 3 eq using activator reagents, DIC (3.0 eq) and HOAT (3.0 eq) in DMF. The reaction was monitored by ninhydrin color reaction or tetrachlor color reaction. After synthesis completion, the peptide resin was washed with DMF×3, MeOH×3, and then dried under N₂ bubbling overnight. The peptide resin was then treated with 92.500 TFA/2.5% TIS/2.5% EDT/2.5% H₂O for 3h. The peptide was precipitated with cold isopropyl ether and centrifuged (3 min at 3000 rpm). The pellet was washed twice with isopropyl ether and the crude peptide was dried under vacuum for 2 hours and then lyophilised. The lyophilised powder was dissolved in of ACN/H₂O (50:50), and a solution of 100 mM TATA in ACN was added, followed by ammonium bicarbonate in H₂O (1M) and the solution mixed for 1 h. Once the cyclisation was complete, the reaction was quenched with 1M aq. Cysteine hydrochloride (10 eq relative to TATA), then mixed and left to stand for an hour. The solution was lyophilised to afford crude product. The crude peptide was purified by Preparative HPLC and lyophilized to give Bicycle Peptide 1, having amino acid Sequence: (β-Ala)-Sar₁₀-(SEQ ID NO: 1)-CONH₂.

8.0 g of resin was used to generate 2.1 g Bicycle Peptide 1 (99.2% purity; 16.3% yield) as a white solid.

Bicycle Peptide 1 Analytical Data

| Mobile Phase: | A: 0.1% TFA in H2O B: 0.1% TFA in ACN |
|---|---|
| Flow: | 1.0 ml/min |
| Column: | Gemini-NX C18 5um 110A 150*4.6 mm |
| Instrument: | Agilent 1200 HPLC-BE(1-614) |
| Method: | 15-45% B over 20 minutes, then 3 min 95% B |
| Retention Time: | 11.31 min |
| LCMS (ESI): | m/z 1061.8 [M + 3H]³⁺, 796.5 [M + 4H]⁴⁺ |
| Peptide mw | 3183.68 |

### Preparation of MMAE-PABC-Cit-Val-Glutarate-NHS

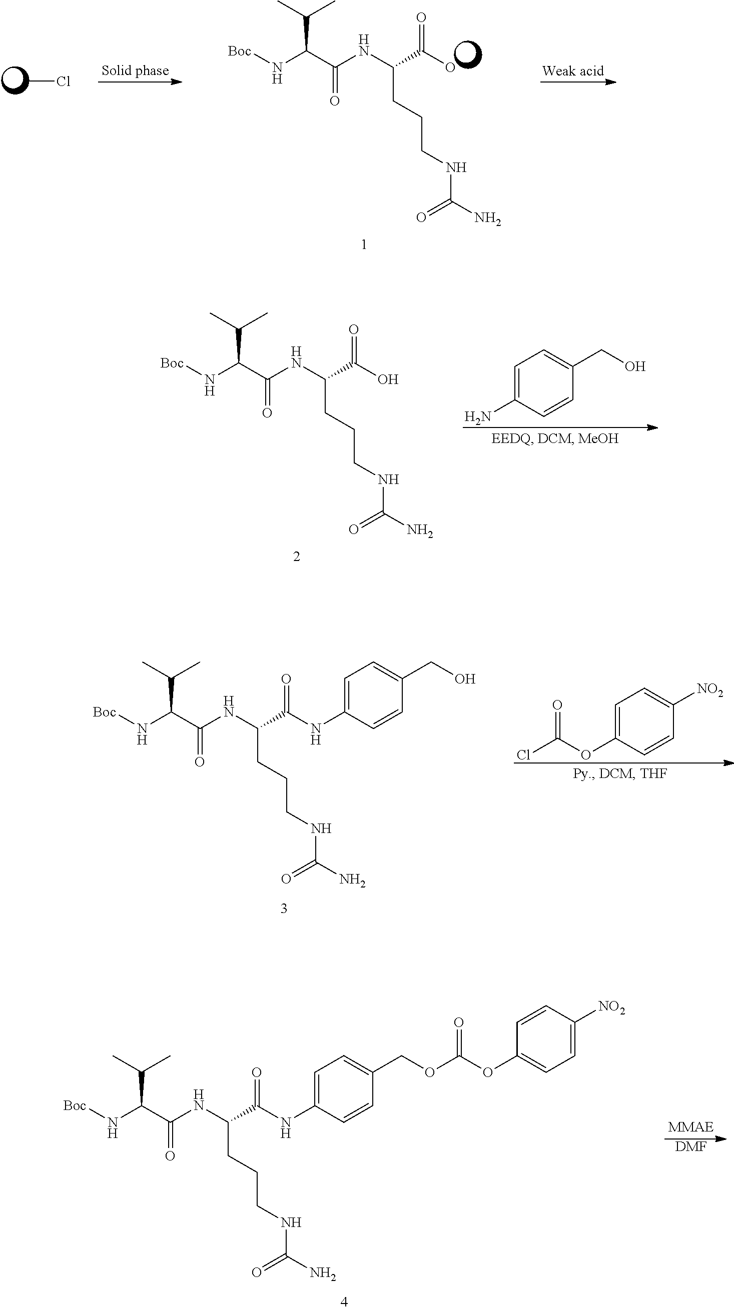

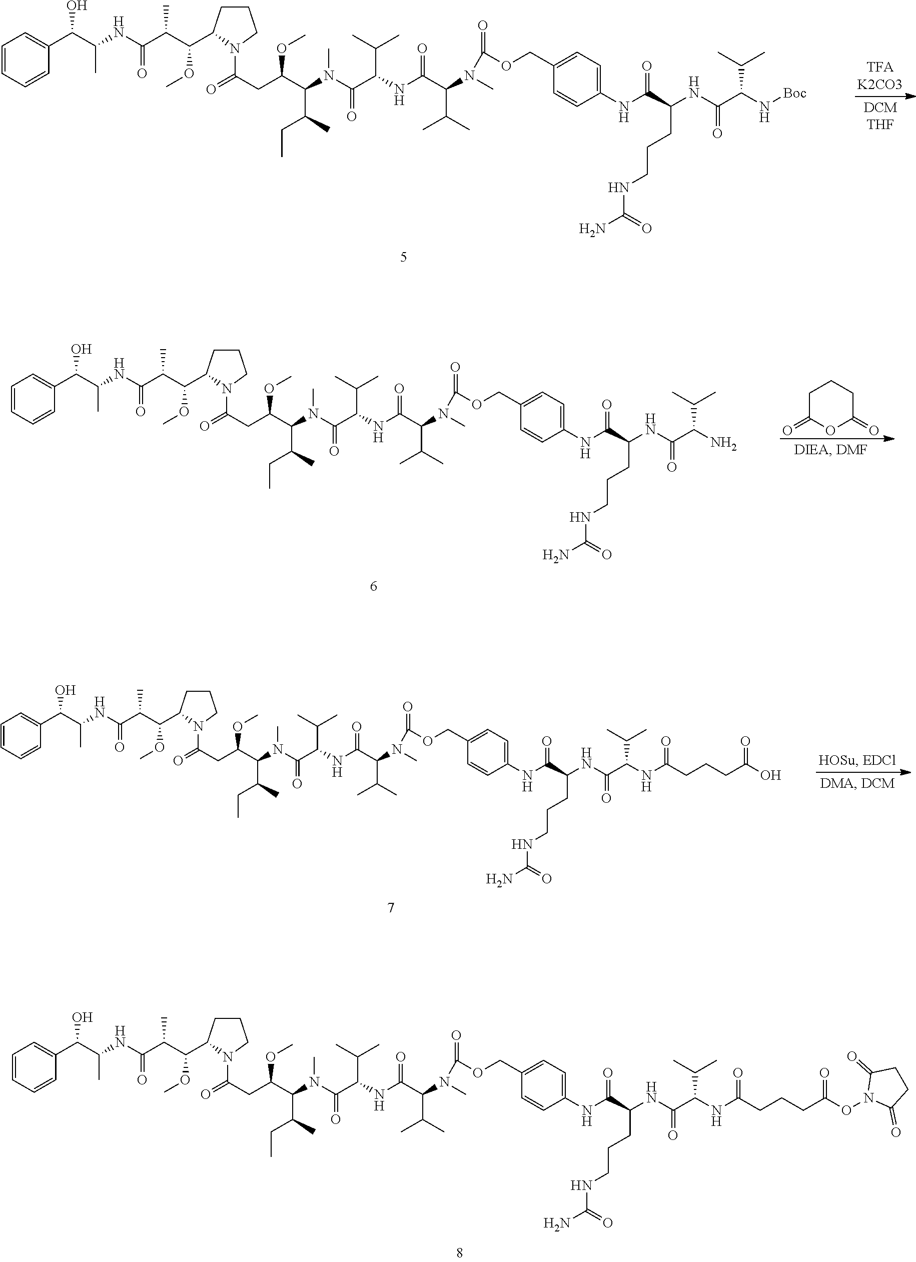

### Preparation of Compound 2

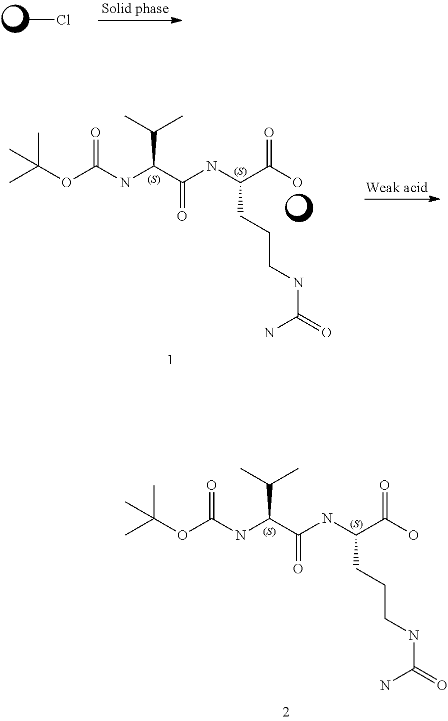

The peptide was synthesized by solid phase synthesis. 50 g CTC Resin (sub: 1.0 mmol/g) was used. To a mixture containing CTC Resin (50 mmol, 50 g, 1.0 mmol/g) and Fmoc-Cit-OH (19.8 g, 50 mmol, 1.0 eq) was added DCM (400 mL), then DIEA (6.00 eq) was added and mixed for 3 hours. And then MeOH (50 mL) was added and mixed for 30 min for capping. 20% piperidine in DMF was used for deblocking. Boc-Val-OH (32.5 g, 150 mmol, 3 eq) was coupled with 3 eq using HBTU (2.85 eq) and DIPEA (6.0 eq) in DMF (400 mL). The reaction was monitored by ninhydrin colour reaction test. After synthesis completion, the peptide resin was washed with DMF×3, MeOH×3, and then dried under N₂ bubbling overnight. After that the peptide resin was treated with 20% HFIP/DCM for 30 min for 2 times. The solution was removed on a rotary evaporator to give the crude. The crude peptide was dissolved in ACN/H2O, then llyophilized twice to give the peptide product (17.3 g crude).

| LCMS (ESI): | m/z 374.9 [M + H]⁺ |
|---|---|
| Molecular weight | 374.44 |

### Preparation of Compound 3

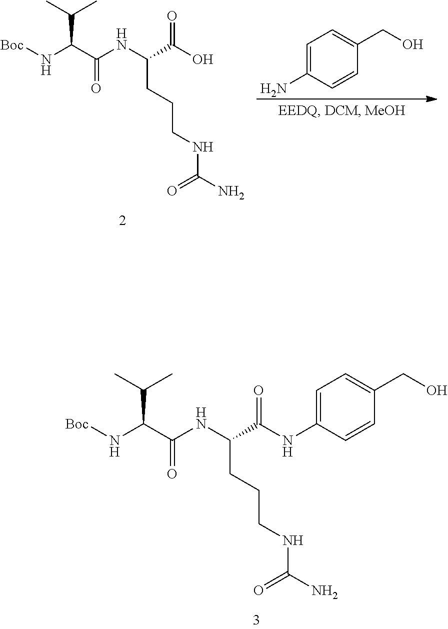 [SMILES: CC(C)[C@H](NC(=O)OC(C)(C)C)C(=O)N[C@@H](CCCNC(N)=O)C(=O)O ; Nc1ccc(CO)cc1 ; CC(C)[C@H](NC(=O)OC(C)(C)C)C(=O)N[C@@H](CCCNC(N)=O)C(=O)Nc1ccc(CO)cc1]

A solution of Compound 2 (4.00 g, 10.68 mmol, 1.00 eq) in DCM (40.00 mL) and MeOH (20.00 mL) was stirred at room temperature, then (4-aminophenyl)methanol (1.58 g, 12.82 mmol, 1.20 eq) and EEDQ (5.28 g, 21.37 mmol, 2.00 eq) were added and the mixture stirred in the dark for 9 hrs. TLC (dichloromethane/methanol=5/1, Rf=0.56) indicated one new spot had formed. The reaction mixture was concentrated under reduced pressure to remove solvent. The resulting residue was purified by flash silica gel chromatography (ISCO®; 120 g SepaFlash® Silica Flash Column, Eluent of 0˜20% MeOH/DCM @ 80 mL/min). Compound 3 (3.00 g, 6.26 mmol, 58.57% yield) was obtained as a white solid.

| LCMS (ESI): | m/z 480.1 [M + H]⁺ |
|---|---|
| Molecular weight | 479.58 |

### Preparation of Compound 4

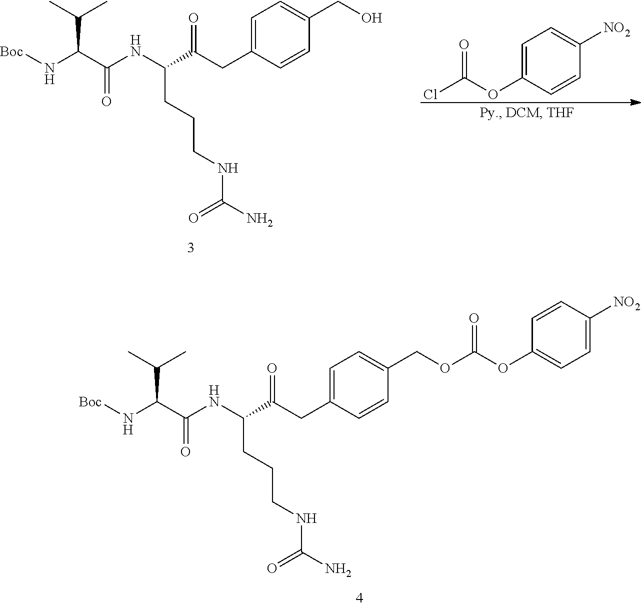 [SMILES: CC(C)[C@H](NC(=O)OC(C)(C)C)C(=O)N[C@@H](CCCNC(N)=O)C(=O)Cc1ccc(CO)cc1 ; O=C(Cl)Oc1ccc([N+](=O)[O-])cc1 ; CC(C)[C@H](NC(=O)OC(C)(C)C)C(=O)N[C@@H](CCCNC(N)=O)C(=O)Cc1ccc(COC(=O)Oc2ccc([N+](=O)[O-])cc2)cc1]

To a solution of Compound 3 (3.00 g, 6.26 mmol, 1.00 eq) in anhydrous THF (35.00 mL) and anhydrous DCM (15.00 mL) was added (4-nitrophenyl) chloroformate (6.31 g, 31.30 mmol, 5.00 eq) and pyridine (2.48 g, 31.30 mmol, 2.53 mL, 5.00 eq), and the mixture was stirred at 25° C. for 5 hrs. TLC (dichloromethane/methanol=10/1, Rf=0.55) indicated a new spot had formed. The reaction mixture was filtered, and the filtrate was concentrated under reduced pressure to give a residue. The residue was purified by flash silica gel chromatography (ISCO®; 120 g SepaFlash® Silica Flash Column, Eluent of 0˜10% DCM/MeOH@ 80 mL/min). Compound 4 (2.00 g, 3.10 mmol, 49.56% yield) was obtained as a white solid.

| LCMS (ESI): | m/z 667.3 [M + Na]⁺ |
|---|---|
| Molecular weight | 644.68 |

### Preparation of Compound 5

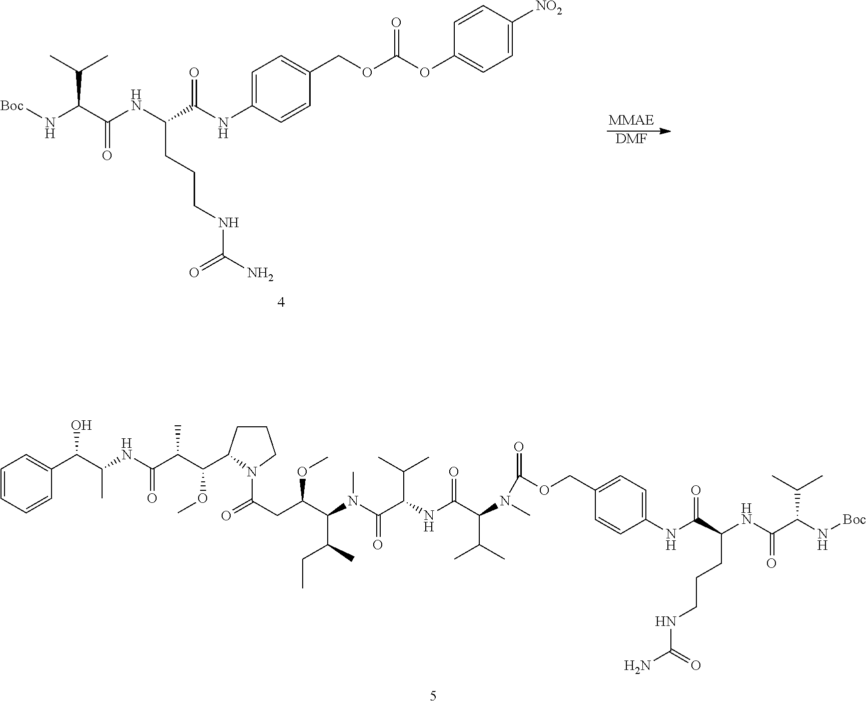

A mixture of Compound 4 (278.43 mg, 387.80 pmol, 1.00 eq and DIEA (501.19 mg, 3.88 mmol, 677.29 μL, 10.00 eq in DMF (5.00 mL) was stirred under nitrogen for 10 min. MMAE (250.00 mg, 387.80 pmol, 1.00 eq) and HOBt (52.40 mg, 387.80 pmol, 1.00 eq) were added and the mixture was stirred at 0° C. under nitrogen for 20 min and stirred at 30° C. for additional 18 hrs. LC-MS showed one main peak with desired mass was detected. The resulting mixture was purified by flash C18 gel chromatography (ISCO®; 130 g SepaFlash® C18 Flash Column, Eluent of 0˜50% MeCN/H₂O @ 75 mL/min). Compound 5 (190.00 mg, 155.29 μmol, 40.04% yield) was obtained as a white solid.

| LCMS (ESI): | m/z 1223.4 [M + H]⁺ |
|---|---|
| Molecular weight | 1223.57 |

### Preparation of Compound 6

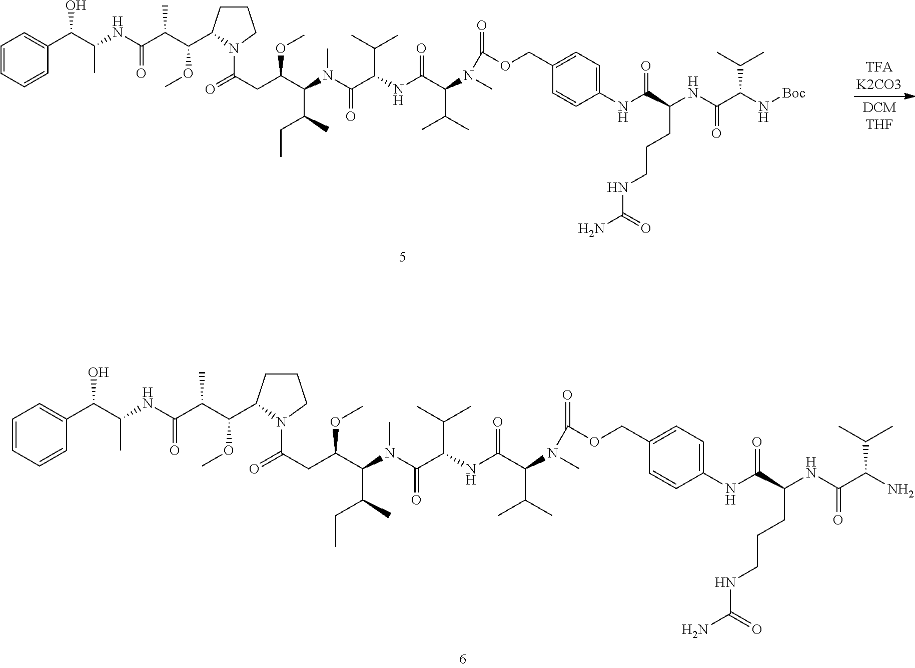 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)OC(C)(C)C)C(C)C)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C]

To a solution of Compound 5 (170.00 mg, 138.94 μmol, 1.00 eq in DCM (2.70 mL) was added 2,2,2-trifluoroacetic acid (413.32 mg, 3.62 mmol, 268.39 μL, 26.09 eq), and the mixture was stirred at 25° C. for 1 hr. LC-MS showed Compound 5 was consumed completely. The mixture was concentrated under reduced pressure to give a residue. The residue was dissolved in THF (10.00 mL) and was added K₂CO₃ (192.03 mg, 1.39 mmol, 10.00 eq), the mixture was stirred at room temperature for additional 3 hrs. LC-MS showed one main peak with desired mass was detected. The resulting reaction mixture was concentrated under reduced pressure to remove solvent to give a residue. The residue was purified by flash C18 gel chromatography (ISCO®; 130 g SepaFlash® C18 Flash Column, Eluent of 0˜50% MeCN/H₂O @ 75 mL/min). Compound 6 (110.00 mg, 97.92 μmol, 70.48% yield) was obtained as a white solid.

| LCMS (ESI): | m/z 1123.4 [M + H]⁺ |
|---|---|
| Molecular weight | 1123.45 |

### Preparation of Compound 7

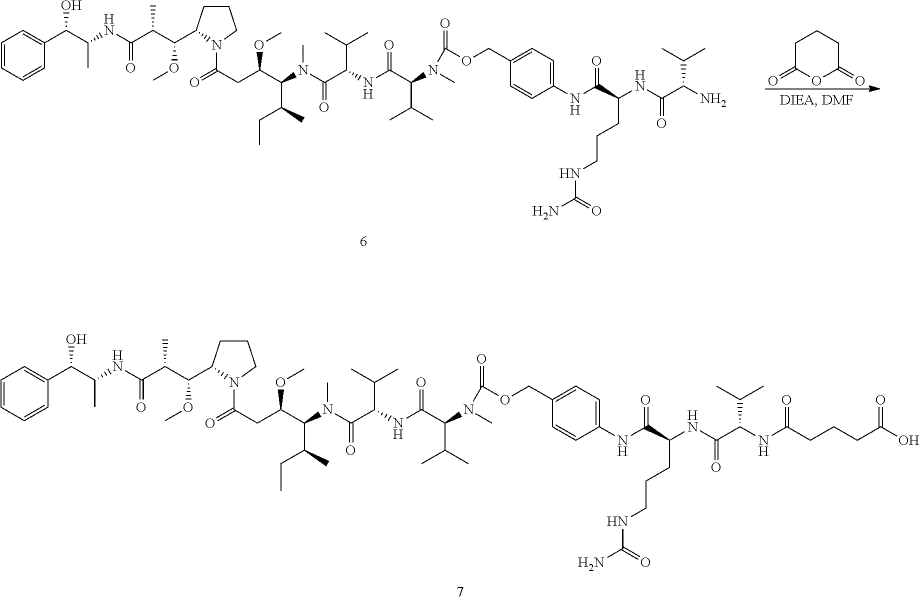 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C ; O=C1CCCC(=O)O1 ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)O)C(C)C)cc1)C(C)C]

To a solution of Compound 6 (110.00 mg, 97.92 μmol, 1.00 eq) in DMA (5 mL), DIEA (25.31 mg, 195.83 μmol, 34.20 μL, 2.00 eq) and tetrahydropyran-2,6-dione (22.34 mg, 195.83 μmol, 2.00 eq). The mixture was stirred at room temperature for 18 hrs. LC-MS showed Compound 6 was consumed completely and one main peak with desired mass was detected. The reaction mixture was purified by flash C18 gel chromatography (ISCO®; 130 g SepaFlash® C18 Flash Column, Eluent of 0˜50% MeCN/H₂O @ 75 mL/min). Compound 7 (100.00 mg, 80.81 μmol, 82.53% yield) was obtained as a white solid.

| LCMS (ESI): | m/z 1237.4 [M + H]⁺ |
|---|---|
| Molecular weight | 1236.74 |

### Preparation of Compound 8 (MMAE-PABC-Cit-Val-Glutarate-NHS)

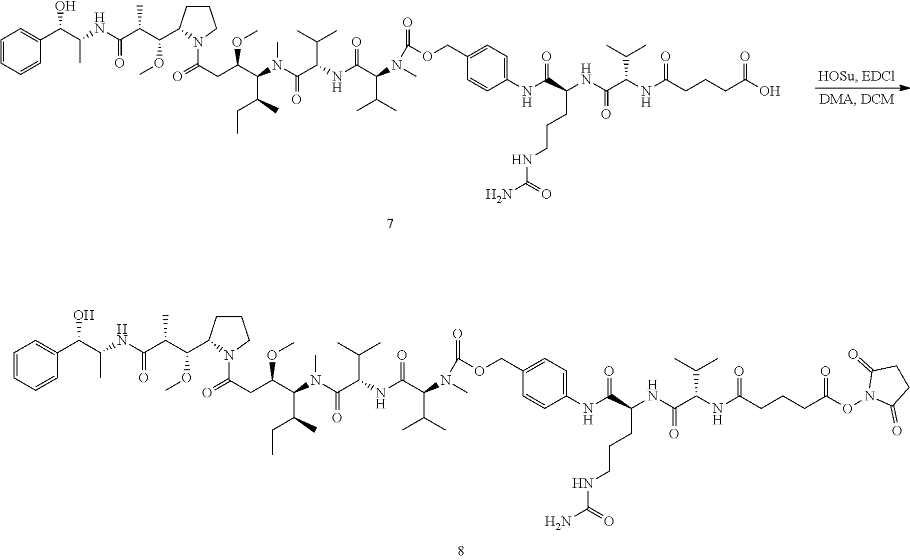 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)O)C(C)C)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)ON2C(=O)CCC2=O)C(C)C)cc1)C(C)C]

To a solution of Compound 7 (100.00 mg, 80.81 μmol, 1.00 eq in DMA (4.5 mL) and DCM (1.5 mL) was added 1-hydroxypyrrolidine-2,5-dione (27.90 mg, 242.42 μmol, 3.00 eq under N₂, the mixture was stirred at 0° C. for 30 min EDCI (46.47 mg, 242.43 μmol, 3.00 eq was added in the mixture, and the mixture was stirred at 25° C. for additional 16 hrs. LC-MS showed Compound 7 was consumed completely and one main peak with desired mass was detected. The reaction mixture was purified by flash C18 gel chromatography (ISCO®; 130 g SepaFlash® C18 Flash Column, Eluent of 0˜50% MeCN/H₂O @ 75 mL/min). Compound 8 (90.00 mg, 60.69 μmol, 75.11% yield) was obtained as a white solid.

| LCMS (ESI): | m/z 1334.5 [M + H]⁺ |
|---|---|
| Molecular weight | 1334.62 |

### Preparation of BT5528

To a solution of Bicycle Peptide 1 (1.0-1.3 eq) in DMA was added DIEA (3 eq) and compound 8 (1 eq). The mixture was stirred at 25° C. for 18 hr. The reaction was monitored by LC-MS and once complete, was directly purified by preparative HPLC.

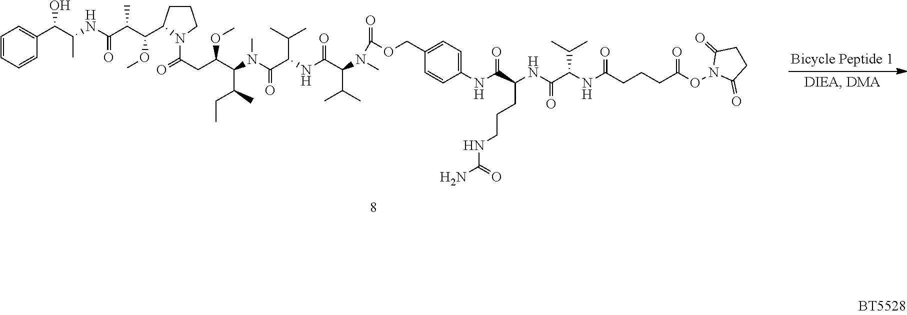 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)ON2C(=O)CCC2=O)C(C)C)cc1)C(C)C]

Bicycle Peptide 1 (71.5 mg, 22.48 μmol) was used as the bicycle reagent. BT5528 (40.9 mg, 9.05 μmol, 40.27% yield, 97.42% purity) was obtained as a white solid.

BT5528 Analytical Data

| Mobile Phase: | A: 0.1% TFA in H2O B: 0.1% TFA in ACN |
|---|---|
| Flow: | 1.0 ml/min |
| Column: | Gemini-NX C18 5um 110A 150*4.6 mm |
| Instrument: | Agilent 1200 HPLC-BE(1-614) |
| Method: | 28-68% B over 30 minutes, then 3 min 95% B |
| Retention Time: | 11.35 min |
| LCMS (ESI): | m/z 1468.1 [M + 3H]3⁺, 1101.2 [M + 4H]4⁺, |
| | 881.3 [M + 5H]5⁺ |
| Peptide mw | 4404.2 |

BCY10188 can be synthesized similarly, for example, by coupling Bicycle Peptide 1 with the corresponding MMAF intermediate MMAF-PABC-Cit-Val-Glutarate-NHS.

### Example 2: In Vivo Efficacy Study of BT5528 and BCY10188 in Treatment of PC-3 Xenograft in BALB/c Nude Mice

### 1. Study Objective

The objective of the research was to evaluate the in vivo anti-tumor efficacy of BT5528 and BCY10188 in treatment of PC-3 xenograft model in BALB/c nude mice.

### 2. Experimental Design

| | | Dose | | Dosing | |
|---|---|---|---|---|---|
| Group | Treatment | (mg/kg) | N | Route | Schedule |
| 1 | Vehicle | — | 5 | i.v. | qw × 4 weeks |
| 2 | BT5528 | 3 | 5 | i.v. | qw × 4 weeks |
| 3 | BT5528 | 1 | 5 | i.v. | qw × 4 weeks |
| 4 | BT5528 | 0.33 | 5 | i.v. | qw × 4 weeks |
| 5 | BT5528 | 0.11 | 5 | i.v. | qw × 4 weeks |
| 6 | BCY10188 | 3 | 5 | i.v. | qw × 4 weeks |
| 7 | BCY10188 | 1 | 5 | i.v. | qw × 4 weeks |
| 8 | BCY10188 | 0.33 | 5 | i.v. | qw × 4 weeks |
| 9 | BCY10188 | 0.11 | 5 | i.v. | qw × 4 weeks |
| 10 | BT5528 | 1 | 5 | i.v. | qw × 2 weeks |
| | | | | | monitor until D28 |
| 11 | BT5528 | 1 | 5 | i.v. | qw × 2 weeks |
| | | | | 1 h infusion | monitor until D28 |
| 12 | BT5528 | 1 | 5 | sc. 24 h | qw × 2 weeks |
| | | | | minipump | monitor until D28 |

Note:
N: animal number; Dosing volume: adjust dosing volume based on body weight 10 μl/g.

### 3. Materials

### 3.1. Animals and Housing Condition

### 3.1.1. Animals

Species: Mus Musculus

Strain: BALB/c nude

Age: 6-8 weeks

Sex: female

Body weight: 18-22 g

Number of animals: 60 mice plus spare

### 3.1.2. Housing Condition

The mice were kept in individual ventilation cages at constant temperature and humidity with 5 animals in each cage.

Temperature: 20˜26° C.

Humidity 40-70%.

Cages: Made of polycarbonate. The size is 300 mm×180 mm×150 mm. The bedding material is corn cob, which is changed twice per week.

Diet: Animals had free access to irradiation sterilized dry granule food during the entire study period.

Water: Animals had free access to sterile drinking water.

Cage identification: The identification labels for each cage contained the following information: number of animals, sex, strain, the date received, treatment, study number, group number and the starting date of the treatment.

Animal identification: Animals were marked by ear coding.

### 3.2. Test and Positive Control Articles

Product identification: BT5528

Physical description: Lyophilised powder

Molecular weight: 4402.17

Purity: 98.5%

Package and storage condition: stored at −80° C.

Product identification: BCY10188

Physical description: Lyophilised powder

Molecular weight: 4416.15

Purity: 98.39%

Package and storage condition: stored at −80° C.

### 4. Experimental Methods and Procedures

### 4.1 Cell Culture

The cells growing in an exponential growth phase were harvested and counted for tumor inoculation.

### 4.2. Tumor Inoculation

Each mouse was inoculated subcutaneously at the right flank with PC-3 tumor cells (10×1\hat{ }6) in 0.2 ml of PBS for tumor development. 60 animals were randomized when the average tumor volume reached 464 mm³. The test article administration and the animal numbers in each group were shown in the experimental design table.

### 4.3. Testing Article Formulation Preparation

| | Dose | |
|---|---|---|
| Treatment | (mg/ml) | Formulation |
| Vehicle | — | 25 mM Histidine, 10% sucrose |
| BT5528 | 0.3 | Dissolve 8.28 mg BT5528 in 27.186 ml |
| | | Histidine buffer. |
| | 0.1 | Dilute 6 ml 0.3 mg/ml BT5528 with 12 ml |
| | | Histidine buffer. |
| | 0.13 | Dilute 1.56 ml 0.3 mg/ml BT5528 with |
| | | 2.04 ml Histidine buffer. |
| | 0.033 | Dilute 2.376 ml 0.1 mg/ml BT5528 with |
| | | 4.824 ml Histidine buffer. |
| | 0.011 | Dilute 0.792 ml 0.1 mg/ml BT5528 with |
| | | 6.408 ml Histidine buffer. |
| BCY10188 | 0.3 | Dissolve 3.65 mg BCY10188 in 11.970 ml |
| | | Acetate buffer. |
| | 0.1 | Dilute 3.6 ml 0.3 mg/ml BCY10188 with |
| | | 7.2 ml Acetate buffer. |
| | 0.033 | Dilute 2.376 ml 0.1 mg/ml BCY10188 with |
| | | 4.824 ml Acetate buffer. |
| | 0.011 | Dilute 0.792 ml 0.1 mg/ml BCY10188 with |
| | | 6.408 ml Acetate buffer. |

### 4.4. Observations

All the procedures related to animal handling, care and the treatment in the study were performed according to the guidelines. At the time of routine monitoring, the animals were checked for any effects of tumor growth and treatments on normal behavior such as mobility, food and water consumption (by looking only), body weight gain/loss, eye/hair matting and any other abnormal effect as stated in the protocol. Death and observed clinical signs were recorded on the basis of the numbers of animals within each subset.

### 4.5. Intravenous Infusion and Alzet Pump Slow-Release

### 4.5.1 Intravenous Infusion

1) 0.1 mg/mL solution (with 50 mM Acetate 10% sucrose pH 5 buffer) was prepared before the operation.
2) The syringe loaded with dosing solution was fixed on the Syringe Pump Machine, then a needle was bound to the pipe jointed with the syringe.
3) The injection volume (10 ml/kg, calculated based on the bodyweight), injection time (1 h for this study), and infusion rate was set to the Pump, then a quick running was conducted to check the working condition of the whole system.
4) The mouse was fixed comfortably, then the operator injected the syringe needle into the caudal vein, and fixed the mouse tail as well as the needle firmly and stably(no need anesthesia for less than 1 h)
5) Then the infusion was started, and the operator kept monitoring the dosing condition till the end.

### 4.5.2 Alzet Pump Preparation and Embedding

1) 0.130 mg/mL solution (with 50 mM Acetate 10% sucrose pH 5 buffer) was prepared before the operation.
2) The pump was fully filled with the dosing solution (˜200 μl, see the hand book of the Alzet pump 2001D).
3) The animal in group 12 was anesthetized with 80 mg/kg pentobarbital sodium, and the pump was embedded under the skin of left side of mouse.
4) 26 h later, the animals were anesthetized with pentobarbital sodium, and the pump was taken out. (The infusion rate is not stable at the first 1-3 h, thus the pump was taken out at 26 h instead of 24 h to ensure that all solution was pumped-out).

### 4.6. Tumor Measurements and the Endpoints

The major endpoint was to see if the tumor growth could be delayed or mice could be cured. Tumor volume was measured 3 times per week in two dimensions using a caliper, and the volume was expressed in mm³ using the formula: V=0.5 a×b² where a and b are the long and short diameters of the tumor, respectively. The tumor size was then used for calculations of T/C value. The T/C value (in percent) is an indication of antitumor effectiveness; T and C are the mean volumes of the treated and control groups, respectively, on a given day.

TGI was calculated for each group using the formula: TGI (%)=[1−(Tᵢ−T₀)/(Vᵢ−V₀)]×100; Tᵢ is the average tumor volume of a treatment group on a given day, T₀ is the average tumor volume of the treatment group on the day of treatment start, Vᵢ is the average tumor volume of the vehicle control group on the same day with Tᵢ, and V₀ is the average tumor volume of the vehicle group on the day of treatment start.

### 4.7. Statistical Analysis

Summary statistics, including mean and the standard error of the mean (SEM), are provided for the tumor volume of each group at each time point.

Statistical analysis of difference in tumor volume among the groups was conducted on the data obtained at the best therapeutic time point after the final dose.

A one-way ANOVA was performed to compare tumor volume among groups, and when a significant F-statistics (a ratio of treatment variance to the error variance) was obtained, comparisons between groups were carried out with Games-Howell test. All data were analyzed using GraphPad Prism 5.0. P<0.05 was considered to be statistically significant.

### 5. Results

5.1. Body Weight change and Tumor Growth Curve

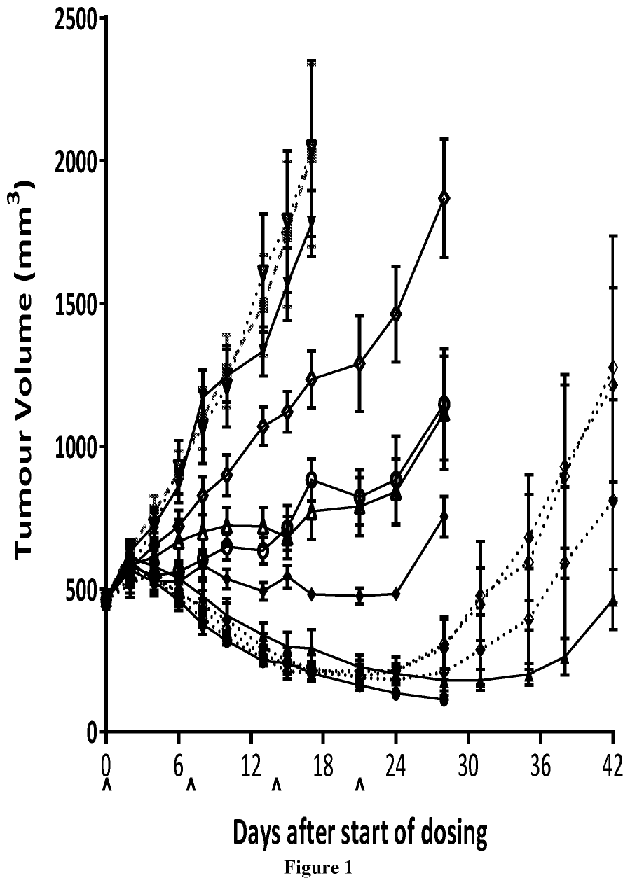

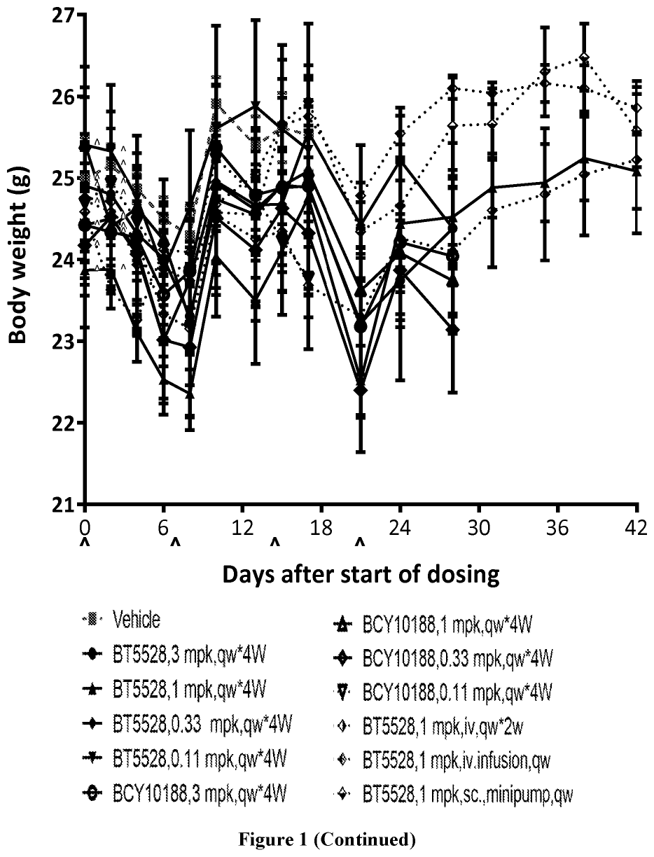

Body weight and tumor growth are shown in FIG. 1.

### 5.2. Tumor Volume Trace

Mean tumor volume over time in female BALB/c nude mice bearing PC-3 xenograft is shown in Table 2-1.

TABLE 2-1
Tumor volume trace over time

| | | Days after the start of treatment | | | | | | | | |
|---|---|---|---|---|---|---|---|---|---|---|
| Gr. | Treatment | 0 | 2 | 4 | 6 | 8 | 10 | 13 | 15 | 17 |
| 1 | Vehicle, qw | 464 ± | 640 ± | 772 ± | 919 ± | 1097 ± | 1264 ± | 1495 ± | 1744 ± | 2019 ± |
| | | 22 | 30 | 54 | 68 | 108 | 128 | 176 | 254 | 318 |
| 2 | BT5528, | 464 ± | 566 ± | 526 ± | 462 ± | 377 ± | 320 ± | 249 ± | 243 ± | 205 ± |
| | 3 mpk, iv | 31 | 59 | 49 | 37 | 36 | 24 | 21 | 21 | 14 |
| | qw * 4 weeks | | | | | | | | | |
| 3 | BT5528, | 463 ± | 610 ± | 579 ± | 538 ± | 476 ± | 408 ± | 341 ± | 299 ± | 293 ± |
| | 1 mpk, iv | 35 | 59 | 56 | 57 | 52 | 61 | 43 | 51 | 66 |
| | qw * 4 weeks | | | | | | | | | |
| 4 | BT5528, | 463 ± | 597 ± | 530 ± | 525 ± | 584 ± | 536 ± | 493 ± | 544 ± | 482 ± |
| | 0.33 mpk, iv | 33 | 44 | 43 | 34 | 43 | 34 | 31 | 40 | 22 |
| | qw * 4 weeks | | | | | | | | | |
| 5 | BT5528, | 463 ± | 634 ± | 725 ± | 861 ± | 1172 ± | 1247 ± | 1333 ± | 1568 ± | 1780 ± |
| | 0.11 mpk, iv | 20 | 39 | 46 | 58 | 96 | 93 | 86 | 127 | 116 |
| | qw * 4 weeks | | | | | | | | | |
| 6 | BCY10188, | 464 ± | 580 ± | 547 ± | 561 ± | 604 ± | 649 ± | 635 ± | 715 ± | 883 ± |
| | 3 mpk, iv | 23 | 35 | 50 | 38 | 31 | 45 | 48 | 79 | 73 |
| | qw * 4 weeks | | | | | | | | | |
| 7 | BCY10188, | 463 ± | 585 ± | 612 ± | 668 ± | 700 ± | 722 ± | 720 ± | 681 ± | 772 ± |
| | 1 mpk, iv | 26 | 66 | 56 | 77 | 63 | 66 | 68 | 74 | 97 |
| | qw * 4 weeks | | | | | | | | | |
| 8 | BCY10188, | 463 ± | 573 ± | 654 ± | 720 ± | 828 ± | 899 ± | 1070 ± | 1121 ± | 1235 ± |
| | 0.33 mpk, iv | 28 | 43 | 44 | 57 | 66 | 72 | 68 | 71 | 99 |
| | qw * 4 weeks | | | | | | | | | |
| 9 | BCY10188, | 464 ± | 567 ± | 722 ± | 926 ± | 1067 ± | 1210 ± | 1607 ± | 1788 ± | 2044 ± |
| | 0.11 mpk, iv | 30 | 50 | 63 | 93 | 128 | 142 | 206 | 248 | 308 |
| | qw * 4 weeks | | | | | | | | | |
| 10 | BT5528, | 464 ± | 572 ± | 529 ± | 496 ± | 427 ± | 361 ± | 269 ± | 239 ± | 214 ± |
| | 1 mpk, iv. | 33 | 34 | 40 | 51 | 41 | 36 | 34 | 29 | 33 |
| | qw * 2 weeks | | | | | | | | | |
| 11 | BT5528, | 463 ± | 538 ± | 539 ± | 467 ± | 399 ± | 330 ± | 260 ± | 213 ± | 209 ± |
| | 1 mpk, iv. | 26 | 50 | 45 | 27 | 33 | 26 | 29 | 26 | 27 |
| | 1 h infusion. | | | | | | | | | |
| | qw * 2 weeks | | | | | | | | | |
| 12 | BT5528, | 464 ± | 517 ± | 64 ± | 480 ± | 443 ± | 393 ± | 298 ± | 249 ± | 223 ± |
| | 1 mpk, sc. | 29 | 45 | 47 | 53 | 53 | 58 | 47 | 47 | 46 |
| | 24 h minipump | | | | | | | | | |
| | qw * 2 weeks | | | | | | | | | |

### 5.3. Tumor Growth Inhibition Analysis

Tumor growth inhibition rate of BT5528 and BCY10188 in the PC-3 xenograft model was calculated based on tumor volume measurements on day 17 after the start of treatment.

TABLE 2-2
Tumor growth inhibition analysis

| | | Tumor | T/C^{b} | TGI | |
|---|---|---|---|---|---|
| Gr | Treatment | Volume (mm3)^{a} | (%) | (%) | P value |
| 1 | Vehicle, qw | 2019 ± 318 | — | — | — |
| 2 | BT5528, 3 mpk, iv | 205 ± 14 | 10.2 | 116.7 | p < 0.001 |
| | qw*4 weeks | | | | |
| 3 | BT5528, 1 mpk, iv | 293 ± 66 | 14.5 | 111.0 | p < 0.001 |
| | qw*4 weeks | | | | |
| 4 | BT5528, 0.33 mpk, iv | 482 ± 22 | 23.9 | 98.8 | p < 0.001 |
| | qw*4 weeks | | | | |
| 5 | BT5528, 0.11 mpk, iv | 1780 ± 116 | 88.1 | 15.3 | p > 0.05 |
| | qw*4 weeks | | | | |
| 6 | BCY10188, 3 mpk, iv | 883 ± 73 | 43.7 | 73.1 | p < 0.001 |
| | qw*4 weeks | | | | |
| 7 | BCY10188, 1 mpk, iv | 772 ± 97 | 38.2 | 80.1 | p < 0.001 |
| | qw*4 weeks | | | | |
| 8 | BCY10188, 0.33 mpk, | 1235 ± 99 | 61.2 | 50.4 | p < 0.01 |
| | iv qw*4 weeks | | | | |
| 9 | BCY10188, 0.11 mpk, | 2044 ± 308 | 101.2 | −1.6 | p > 0.05 |
| | iv qw*4 weeks | | | | |
| 10 | BT5528, 1 mpk, iv. | 214 ± 33 | 10.6 | 116.0 | p < 0.001 |
| | qw *2 weeks | | | | |
| 11 | BT5528, 1 mpk, iv. | 209 ± 27 | 10.3 | 116.4 | p < 0.001 |
| | 1 h infusion qw * | | | | |
| | 2 weeks | | | | |
| 12 | BT5528, 1 mpk, sc | 223 ± 46 | 11.1 | 115.5 | p < 0.001 |
| | 24 h minipump qw | | | | |
| | *2 weeks | | | | |

^{a}Mean ± SEM.
^{b}Tumor Growth Inhibition is calculated by dividing the group average tumor volume for the treated group by the group average tumor volume for the control group (T/C).

### 6. Results Summary and Discussion

In this study, the therapeutic efficacy of BT5528 and BCY10188 in the PC-3 xenograft model was evaluated. The measured tumor volumes of all treatment groups at various time points are shown in FIG. 1 and Tables 2-1 and 2-2.

The mean tumor size of vehicle treated mice reached 2019 mm³ on day 17. BT5528 at 3 mg/kg qw*4 weeks (TV=205 mm³, TGI=116.7%, p<0.001), 1 mg/kg qw*4 weeks (TV=282 mm³, TGI=111.0%, p<0.001) and 0.33 mg/kg qw*4 weeks (TV=482 mm³, TGI=98.8%, p<0.001) produced significant antitumor activity. BT5528 at 0.11 mg/kg qw*4 weeks (TV=1780 mm³, TGI=15.3%, p>0.05) didn't show obvious antitumor activity.

BCY10188 at 3 mg/kg qw*4 weeks (TV=883 mm³, TGI=73.0%, p<0.001), 1 mg/kg qw*4 weeks (TV=772 mm³, TGI=80.1%, p<0.001) and 0.33 mg/kg qw*4 weeks (TV=1235 mm³, TGI=50.4%, p<0.01) produced significant antitumor activity. BCY10188 at 0.11 mg/kg qw*4 weeks (TV=2044 mm³, TGI=−1.6%, p>0.05) didn't show any antitumor activity.

BT5528 at 1 mg/kg administered via intravenous bolus (TV=214 mm³, TGI=116.0%, p<0.001), intravenous infusion (TV=209 mm³, TGI=116.4%, p<0.001) or subcutaneous ALZET pump (TV=223 mm³, TGI=115.5%, p<0.001) showed comparable anti-tumor activity. The tumors in those groups showed obvious relapse 2 weeks later after ceasing the treatment.

In this study, animals were supplied with nutritional support (sunflower seeds) to reverse the body weight loss associated with PC-3 tumor growth induced cachexia.

### Example 3: In Vivo Efficacy Study of Test Articles in Treatment of PC-3 Xenograft in Balb/c Nude Mice

### 1. Study Objective

The objective of the research is to evaluate the in vivo anti-tumor efficacy of test articles in treatment of PC-3 xenograft in Balb/c nude mice.

### 2. Experimental Design

| | | Dose | | Dosing | |
|---|---|---|---|---|---|
| Group | Treatment | (mg/kg) | N^{a} | Route | Schedule |
| 1 | Vehicle | — | 4 | i.v. | qw × 4 weeks |
| 2 | BT5528 | 0.167 | 4 | i.v. | qw × 4 weeks |
| 3^{b} | BT5528 | 0.5 | 4 | i.v. | qw × 4 weeks |
| 4 | BT5528 | 1.5 | 4 | i.v. | qw × 4 weeks |
| 5^{b} | BT5528 | 0.5 | 4 | i.v. | q2w × 2 weeks |
| 6^{b} | BT5528 | 1.5 | 4 | i.v. | q2w × 2 weeks |
| 7 | Non-binding BTC | 0.167 | 4 | i.v. | qw × 4 weeks |
| 8 | Non-binding BTC | 0.5 | 4 | i.v. | qw × 4 weeks |
| 9 | Non-binding BTC | 1.5 | 4 | i.v. | qw × 4 weeks |
| 10 | EphA2-ADC | 0.33 | 4 | i.v. | qw × 4 weeks |
| 11 | EphA2-ADC | 1 | 4 | i.v. | qw × 4 weeks |
| 12 | EphA2-ADC | 3 | 4 | i.v. | qw × 4 weeks |
| 13^{c} | Docetaxel | 15 | 4 | i.v. | qw × 4 weeks |

^{a}N, the number of animals in each group.
^{b}After 4 weeks' treatment demonstrated in the experimental design table, the mice of group 3, 5 and 6 were treated with BT5528 1.5 mg/kg qw from day 52 during the monitoring schedule.
^{c}Due to the severe body weight loss of the Docetaxel treated mice after the first dosing, the treatment was suspended for 2 weeks, then a lower dosage (Docetaxel, 10 mg/kg) was performed on day 28. After that, the mice were treated with BT5528 1.5 mg/kg qw from day 42 to day 70.

### 3. Materials

### 3.1. Animals and Housing Condition

### 3.1.1. Animals

Species: Mus Musculus

Strain: Balb/c nude

Age: 6-8 weeks

Sex: male

Body weight: 18-22 g

Number of animals: 52 mice plus spare

### 3.1.2. Housing Condition

The mice were kept in individual ventilation cages at constant temperature and humidity with 4 animals in each cage.

Temperature: 20˜26° C.

Humidity 40-70%.

Cages: Made of polycarbonate. The size is 300 mm×180 mm×150 mm. The bedding material is corn cob, which is changed twice per week.

Diet: Animals had free access to irradiation sterilized dry granule food during the entire study period.

Water: Animals had free access to sterile drinking water.

Cage identification: The identification labels for each cage contained the following information: number of animals, sex, strain, the date received, treatment, study number, group number and the starting date of the treatment.

Animal identification: Animals were marked by ear coding.

### 3.2. Test and Positive Control Articles

Product identification: BT5528

Physical description: Lyophilised powder

Molecular weight: 4402.17

Purity: 98.6%

Product identification: Non-binding BTC (as a negative control)

Physical description: Lyophilised powder

Molecular weight: 4173.85

Purity: 97.7%

### 4. Experimental Methods and Procedures

### 4.1. Cell Culture

The tumor cells were maintained in F-12K medium supplemented with 10% heat inactivated fetal bovine serum at 37° C. in an atmosphere of 5% CO2 in air. The tumor cells were routinely subcultured twice weekly. The cells growing in an exponential growth phase were harvested and counted for tumor inoculation.

### 4.2. Tumor Inoculation

Each mouse was inoculated subcutaneously at the right flank with PC-3 tumor cells (10×10⁶) in 0.2 ml of PBS for tumor development. 52 animals were randomized when the average tumor volume reached 454 mm³. The test article administration and the animal numbers in each group were shown in the experimental design table.

### 4.3. Testing Article Formulation Preparation

| Test | | Conc. | |
|---|---|---|---|
| article | Purity | (mg/ml) | Formulation |
| Vehicle | — | — | 25 mM Histidine pH 7 10% sucrose |
| Non- | 97.7% | 1 | Dissolve 6.23 mg a Non-binding BTC in 6.087 ml Histidine |
| binding | | | buffer¹ |
| BTC | | 0.3 | Dilute 300 μl 1 mg/ml a Non-binding BTC stock with 700 μl |
| | | | Histidine buffer |
| | | 0.15 | Dilute 600 μl 0.3 mg/ml a Non-binding BTC stock with |
| | | | 600 μl Histidine buffer |
| | | 0.05 | Dilute 200 μl 0.3 mg/ml a Non-binding BTC stock with |
| | | | 1000 μl Histidine buffer |
| | | 0.0167 | Dilute 66.7 μl 0.3 mg/ml a Non-binding BTC stock with |
| | | | 1133.3 μl Histidine buffer |
| BT5528 | 98.6% | — | 50 mM Acetate 10% sucrose pH 5 |
| | | 1 | Dissolve 2.70 mg BT5528 in 2.662 ml Acetate buffer |
| | | 0.3 | Dilute 300 μl 1 mg/ml BT5528 stock with 700 μl Acetate |
| | | | buffer² |
| | | 0.15 | Dilute 600 μl 0.3 mg/ml BT5528 stock with 600 μl Acetate |
| | | | buffer |
| | | 0.05 | Dilute 200 μl 0.3 mg/ml BT5528 stock with 1000 μl Acetate |
| | | | buffer |
| | | 0.0167 | Dilute 66.7 p10.3 mg/ml BT5528 stock with 1133.3 μl Acetate |
| | | | buffer |
| EphA2- | — | — | 25 mM Histidine pH 5.5 |
| ADC | | 0.033 | Dilute 9.3 μl 4.24 mg/ml EphA2-ADC stock with 1191 μl His |
| | | | buffer |
| | | 0.1 | Dilute 28 μl 4.24 mg/ml EphA2-ADC stock with 1172 μl His |
| | | | buffer |
| | | 0.3 | Dilute 84.9 μl 4.24 mg/ml EphA2-ADC stock with 1115 μl |
| | | | His buffer |
| Docetaxel | — | 10 | Mix 0.5 ml 20 mg Docetaxel with 1.5 ml buffer |
| | | 1.5 | Dilute 180 μl 10 mg/ml Docetaxel stock with 1020 μl saline |
| | | | buffer |

¹25 mM Histidine pH 7 10% sucrose
²50 mM Acetate 10% sucrose pH 5
³25 mM Histidine pH 5.5

### 4.4. Observations

All the procedures related to animal handling, care and the treatment in the study were performed according to the guidelines following the guidance of the Association for Assessment and Accreditation of Laboratory Animal Care (AAALAC). At the time of routine monitoring, the animals were daily checked for any effects of tumor growth and treatments on normal behavior such as mobility, food and water consumption (by looking only), body weight gain/loss, eye/hair matting and any other abnormal effect as stated in the protocol. Death and observed clinical signs were recorded on the basis of the numbers of animals within each subset.

### 4.5. Tumor Measurements and the Endpoints

The major endpoint was to see if the tumor growth could be delayed or mice could be cured. Tumor volume was measured three times weekly in two dimensions using a caliper, and the volume was expressed in mm3 using the formula: V=0.5 a×b² where a and b are the long and short diameters of the tumor, respectively. The tumor size was then used for calculations of T/C value. The T/C value (in percent) is an indication of antitumor effectiveness; T and C are the mean volumes of the treated and control groups, respectively, on a given day.

TGI was calculated for each group using the formula: TGI (%)=[1−(Tᵢ−T₀)/(Vᵢ−V₀)]×100; Tᵢ is the average tumor volume of a treatment group on a given day, T₀ is the average tumor volume of the treatment group on the day of treatment start, Vᵢ is the average tumor volume of the vehicle control group on the same day with Tᵢ, and V₀ is the average tumor volume of the vehicle group on the day of treatment start.

### 4.6. Statistical Analysis

Summary statistics, including mean and the standard error of the mean (SEM), were provided for the tumor volume of each group at each time point.

Statistical analysis of difference in tumor volume among the groups was conducted on the data obtained at the best therapeutic time point after the final dose.

A one-way ANOVA was performed to compare tumor volume among groups, and when a significant F-statistics (a ratio of treatment variance to the error variance) was obtained, comparisons between groups were carried out with Games-Howell test. All data were analyzed using GraphPad 5.0. P<0.05 was considered to be statistically significant.

### 5. Results

5.1. Body Weight change and Tumor Growth Curve

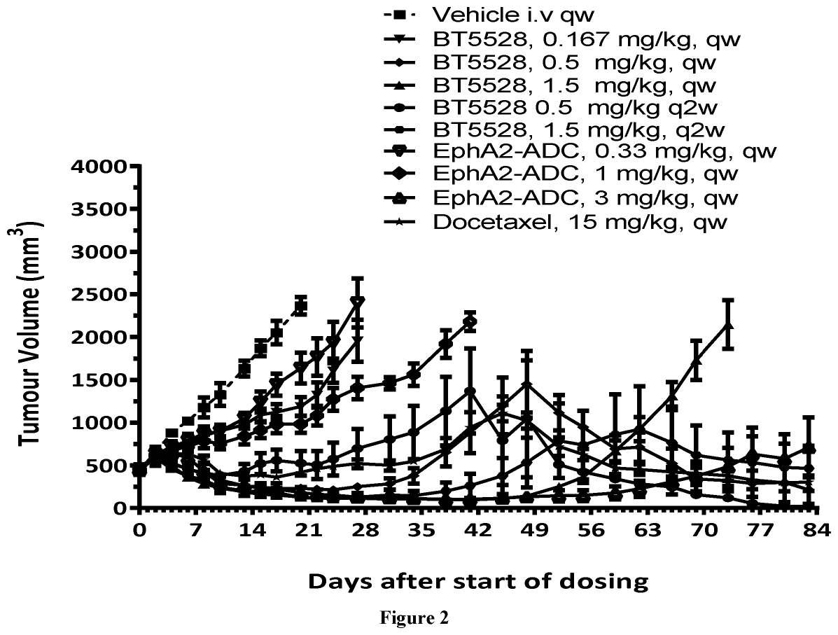

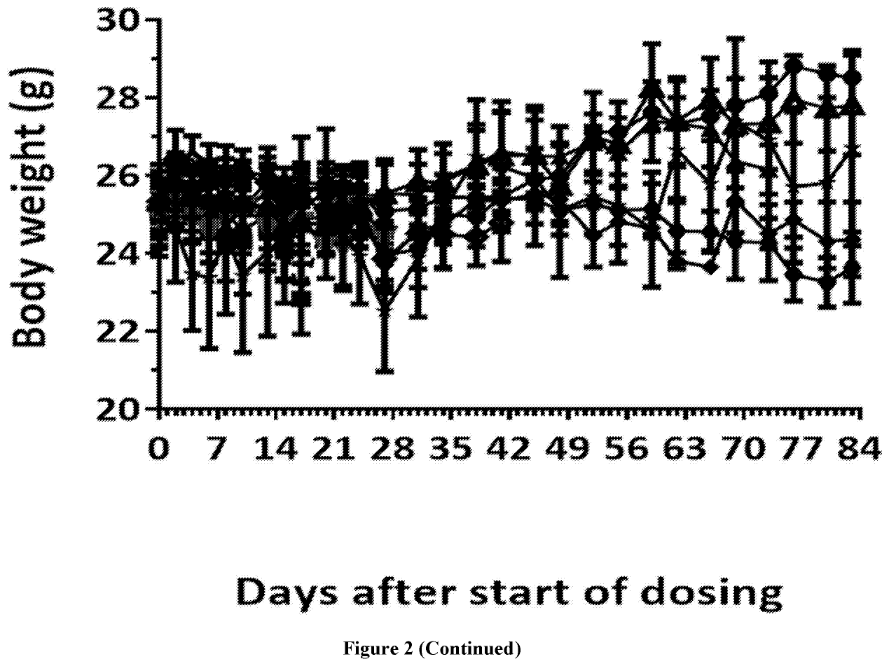

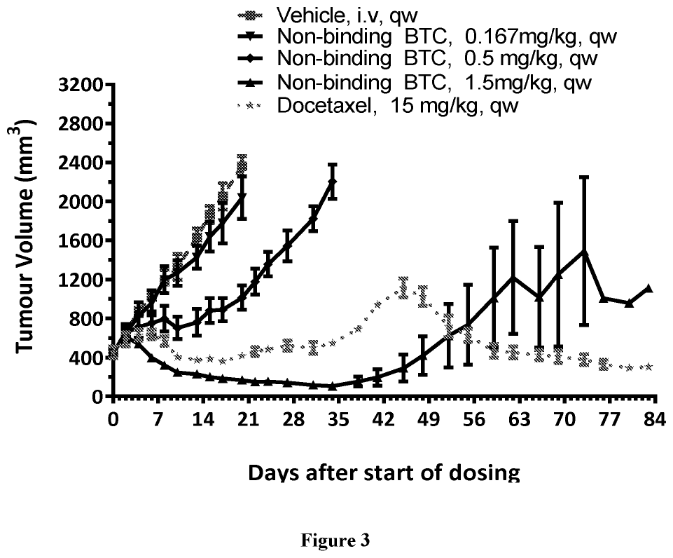

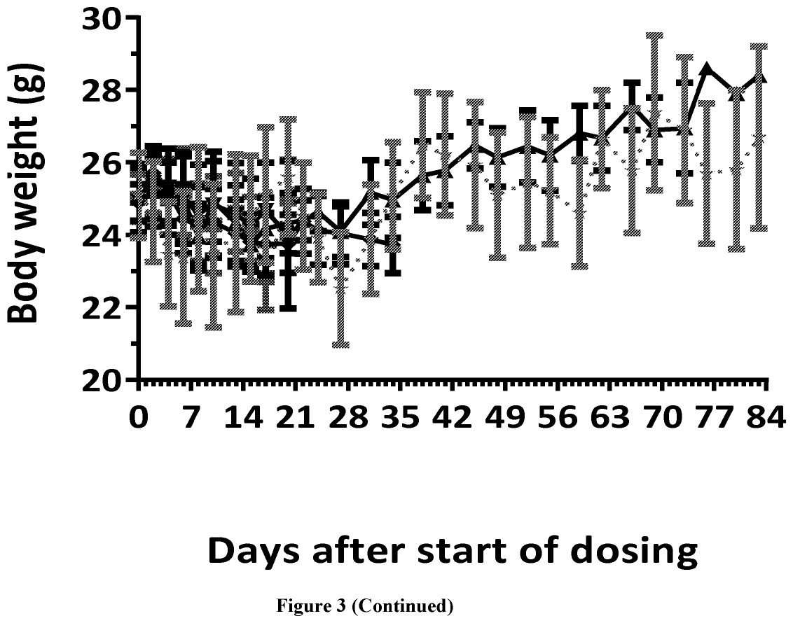

Body weight and tumor growth curve is shown in FIGS. 2 and 3.

### 5.2. Tumor Volume Trace

Mean tumor volume over time in male Balb/c nude mice bearing PC-3 xenograft is shown in Table 3-1.

TABLE 3-1
Tumor volume trace over time (Day 0 to day 20)

| | | Days after the start of treatment | | | | | | | | | |
|---|---|---|---|---|---|---|---|---|---|---|---|
| Gr. | Treatment | 0 | 2 | 4 | 6 | 8 | 10 | 13 | 15 | 17 | 20 |
| 1 | Vehicle, qw | 456 ± | 648 ± | 880 ± | 1022 ± | 1178 ± | 1327 ± | 1631 ± | 1868 ± | 2052 ± | 2364 ± |
| | | 25 | 50 | 23 | 29 | 118 | 133 | 93 | 90 | 139 | 102 |
| 2 | BT5528 | 450 ± | 631 ± | 695 ± | 739 ± | 850 ± | 904 ± | 975 ± | 1089 ± | 1124 ± | 1188 ± |
| | 0.167 mpk, qw | 33 | 55 | 78 | 39 | 68 | 73 | 47 | 74 | 92 | 111 |
| 3 | BT5528 | 451 ± | 622 ± | 519 ± | 460 ± | 398 ± | 329 ± | 260 ± | 249 ± | 231 ± | 234 ± |
| | 0.5 mpk, qw | 47 | 96 | 70 | 55 | 50 | 38 | 33 | 33 | 38 | 42 |
| 4 | BT5528 | 458 ± | 587 ± | 494 ± | 363 ± | 283 ± | 237 ± | 192 ± | 164 ± | 155 ± | 131 ± |
| | 1.5 mpk, qw | 49 | 63 | 54 | 32 | 32 | 24 | 13 | 16 | 20 | 19 |
| 5 | BT5528 | 454 ± | 643 ± | 531 ± | 458 ± | 411 ± | 382 ± | 430 ± | 522 ± | 560 ± | 530 ± |
| | 0.5 mpk, q2w | 37 | 25 | 37 | 33 | 32 | 49 | 88 | 124 | 129 | 147 |
| 6 | BT5528 | 452 ± | 590 ± | 457 ± | 375 ± | 328 ± | 242 ± | 206 ± | 197 ± | 182 ± | 128 ± |
| | 1.5 mpk, q2w | 42 | 75 | 49 | 44 | 47 | 63 | 61 | 62 | 55 | 36 |
| 7 | Non-binding BTC | 453 ± | 651 ± | 824 ± | 960 ± | 1197 ± | 1265 ± | 1428 ± | 1640 ± | 1777 ± | 2038 ± |
| | 0.167 mpk, qw | 42 | 58 | 140 | 127 | 139 | 132 | 119 | 153 | 210 | 218 |
| 8 | Non-binding BTC | 455 ± | 651 ± | 717 ± | 756 ± | 798 ± | 705 ± | 761 ± | 881 ± | 890 ± | 101 ± |
| | 0.5 mpk, qw | 43 | 93 | 112 | 120 | 131 | 115 | 138 | 126 | 119 | 123 |
| 9 | Non-binding BTC | 457 ± | 618 ± | 538 ± | 397 ± | 323 ± | 250 ± | 231 ± | 202 ± | 185 ± | 171 ± |
| | 1.5 mpk, qw | 48 | 58 | 38 | 29 | 29 | 9 | 15 | 10 | 8 | 14 |
| 10 | EphA2-ADC | 457 ± | 636 ± | 712 ± | 792 ± | 870 ± | 900 ± | 1049 ± | 1242 ± | 1443 ± | 1637 ± |
| | 0.33 mpk, qw | 43 | 57 | 70 | 78 | 87 | 58 | 66 | 123 | 129 | 181 |
| 11 | EphA2-ADC | 450 ± | 617 ± | 673 ± | 721 ± | 782 ± | 755 ± | 840 ± | 913 ± | 978 ± | 981 ± |
| | 1 mpk, qw | 49 | 48 | 50 | 61 | 78 | 67 | 93 | 91 | 100 | 100 |
| 12 | EphA2-ADC | 452 ± | 593 ± | 643 ± | 593 ± | 433 ± | 290 ± | 268 ± | 232 ± | 225 ± | 184 ± |
| | 3 mpk, qw | 60 | 98 | 141 | 106 | 103 | 81 | 64 | 60 | 66 | 62 |
| 13 | Docetaxel | 453 ± | 584 ± | 632 ± | 636 ± | 568 ± | 408 ± | 374 ± | 388 ± | 361 ± | 419 ± |
| | 15 mpk, qw | 62 | 72 | 56 | 48 | 50 | 31 | 26 | 36 | 25 | 31 |

### 5.3. Tumor Growth Inhibition Analysis

Tumor growth inhibition rate for test articles in the PC-3 xenograft model was calculated based on tumor volume measurements at day 20 after the start of the treatment.

TABLE 3-2
Tumor growth inhibition analysis

| | | | | | P value |
|---|---|---|---|---|---|
| | | Tumor | | | compared |
| | | Volume | T/C^{b} | TGI | with |
| Gr | Treatment | (mm³)^{a} | (%) | (%) | vehicle |
| 1 | Vehicle, qw | 2364 ± 102 | — | — | — |
| 2 | BT5528, | 1188 ± 111 | 50.2 | 61.4 | p < 0.001 |
| | 0.167 mpk, qw | | | | |
| 3 | BT5528, | 234 ± 42 | 9.9 | 111.4 | p < 0.001 |
| | 0.5 mpk, qw | | | | |
| 4 | BT5528, | 131 ± 19 | 5.5 | 117.2 | p < 0.001 |
| | 1.5 mpk, qw | | | | |
| 5 | BT5528, | 530 ± 147 | 22.4 | 96.0 | p < 0.001 |
| | 0.5 mpk, q2w | | | | |
| 6 | BT5528, | 128 ± 36 | 5.4 | 117.0 | p < 0.001 |
| | 1.5 mpk, q2w | | | | |
| 7 | Non-binding | 2038 ± 8 | 86.2 | 16.9 | p > 0.05 |
| | BTC, | | | | |
| | 0.167 mpk, qw | | | | |
| 8 | Non-binding | 1013 ± 123 | 42.9 | 70.7 | p < 0.001 |
| | BTC, | | | | |
| | 0.5 mpk, qw | | | | |
| 9 | Non-binding | 171 ± 14 | 7.2 | 115.0 | p < 0.001 |
| | BTC, 1.5 | | | | |
| 10 | EphA2-ADC, | 1637 ± 181 | 69.2 | 38.1 | p < 0.001 |
| | 0.33 mpk,qw | | | | |
| 11 | EphA2-ADC, | 981 ± 100 | 41.5 | 72.2 | p < 0.001 |
| | 1 mpk,qw | | | | |
| 12 | EphA2-ADC, | 184 ± 62 | 7.8 | 114.0 | p < 0.001 |
| | 3 mpk,qw | | | | |
| 13 | Docetaxel, | 419 ± 31 | 17.7 | 101.8 | p < 0.001 |
| | 15 mpk,qw | | | | |

^{a}Mean ± SEM.
^{b}Tumor Growth Inhibition is calculated by dividing the group average tumor volume for the treated group by the group average tumor volume for the control group (T/C).

### 6. Results Summary and Discussion

In this study, the therapeutic efficacy of test articles in the PC-3 xenograft model was evaluated. The measured body weights and tumor volumes of all treatment groups at various time points are shown in the FIGS. 2 and 3, and Tables 3-1 and 3-2.

The mean tumor size of vehicle treated mice reached 2364 mm³ on day 20. BT5528 at 0.167 mg/kg, qw (TV=1188 mm³, TGI=61.4%, p<0.001), 0.5 mg/kg, q2w (TV=530 mm³, TGI=96.0%, p<0.001), 0.5 mg/kg, qw (TV=234 mm³, TGI=111.4%, p<0.001) and 1.5 mg/kg, qw (TV=131 mm³, TGI=117.2%, p<0.001) produced significant anti-tumor activity in dose or dose-frequency dependent manner on day 20. BT5528 at 1.5 mg/kg, q2w (TV=128 mm³, TGI=117.0%, p<0.001) produced comparable anti-tumor activity with BT5528 1.5 mg/kg qw. Among them, the mice treated with BT5528, 0.5 mg/kg qw or BT5528, 0.5 mg/kg q2w showed obvious tumor relapse after ceasing the treatment, further treatment with BT5528, 1.5 mg/kg qw from day 52 worked well on the tumor regression. The mice treated with BT5528, 1.5 mg/kg q2w also showed tumor relapse after ceasing the treatment, but further dosing didn't work on complete tumor regression. The mice treated with BT5528, 1.5 mpk qw didn't show any tumor relapse until day 48.

Non-binding BTC at 0.5 mg/kg, qw (TV=1013 mm³, TGI=70.7%, p<0.001) and 1.5 mg/kg, qw (TV=171 mm³, TGI=115.0%, p<0.001) produced significant anti-tumor activity in dose dependent manner on day 20. Non-binding BTC at 0.167 mg/kg, qw (TV=2038 mm³, TGI=16.9%, p>0.05) didn't show any anti-tumor activity. After ceasing the treatment, the mice treated with Non-binding BTC, 1.5 mg/kg qw showed obvious tumor relapse from day 38.

EphA2-ADC at 0.33 mg/kg, qw (TV=1637 mm³, TGI=38.1%, p<0.001), 1 mg/kg, qw (TV=981 mm³, TGI=72.2%, p<0.001) and 3 mg/kg, qw (TV=184 mm³, TGI=114.0%, p<0.001) produced significant anti-tumor activity in dose dependent manner on day 20. The mice treated with EphA2-ADC, 3 mg/kg qw didn't show any tumor relapse until day 59.

Docetaxel at 15 mg/kg, qw (TV=419 mm³, TGI=101.8%, p<0.001) produced significant anti-tumor activity but caused severe animal body weight loss. After ceasing the treatment, the mice showed obvious tumor relapse. The treatment with BT5528, 1.5 mg/kg qw from day 42 worked well on tumor regression of these mice.

### Example 4. In Vivo PK/PD Study of Test Agents in Treatment of PC-3 CDX Model in Balb/c Nude Mice

### 1. Study Objective

The objective of the research is to evaluate the in vivo PK/PD of test agents in treatment of PC-3 CDX model in Balb/c nude mice.

### 2. Experimental Design

| | | | Dose | Time | | Mice #/ |
|---|---|---|---|---|---|---|
| Group | Compound | Route | (mp/kg) | point | Tissue | Timepoint |
| 1 | Vehicle | i.v. | — | 24, 96 h | Plasma (2 aliquots, 50 ul); | 3 |
| 2 | BT5528 | i.v. | 0.167 | 1, 2, 8, 24, | Serum (1 aliquot, 100 ul); | 3 |
| 3 | BT5528 | i.v. | 0.5 | 48, 72, 96 h | Tumor (1 piece frozen for PK, | 3 |
| 4 | BT5528 | i.v. | 1.5 | | 2 pieces frozen for PD/backup, | 3 |
| 5 | Non-binding BTC | i.v. | 0.5 | | 1 piece for FFPE) | 3 |
| 6 | Non-binding BTC | i.v. | 1.5 | | Muscle (~0.2 g quadricep, | 3 |
| 7 | EphA2-ADC | i.v. | 1 | | frozen for back-up) | 3 |
| 8 | EphA2-ADC | i.v. | 3 | | | 3 |

IV injection: Inject the solution by mouse tail vein based on the mouse bodyweight of 10 mL/kg.

### 3. Materials

### 3.1. Animals and Housing Condition

### 3.1.1. Animals

Species: Mus Musculus

Strain: Balb/c nude

Age: 6-8 weeks

Sex: male

Body weight: 19-22 g

Number of animals: 153 plus spare

### 3.1.2. Housing Condition

The mice were kept in individual ventilation cages at constant temperature and humidity with 3 animals in each cage.

Temperature: 20˜26° C.

Humidity 40-70%.

Cages: Made of polycarbonate. The size is 300 mm×180 mm×150 mm. The bedding material is corn cob, which is changed twice per week.

Diet: Animals had free access to irradiation sterilized dry granule food during the entire study period.

Water: Animals had free access to sterile drinking water.

Cage identification: The identification labels for each cage contained the following information: number of animals, sex, strain, the date received, treatment, study number, group number and the starting date of the treatment.

Animal identification: Animals were marked by ear coding.

### 3.2. Test Articles

Product identification: BT5528

Physical description: Lyophilised powder

Molecular weight: 4402.17, purity=98.60%

Package and storage condition: stored at −80° C.

Product identification: non-binding BTC (as a negative control)

Physical description: Lyophilised powder

Molecular weight: 4173.85, purity=96.10%

Package and storage condition: stored at −80° C.

Product identification: EphA2-ADC

Physical description: 4.24 mg/mL solution

Package and storage condition: stored at −80° C.

### 4. Experimental Methods and Procedures

### 4.1. Cell Culture

The PC-3 tumor cells were maintained in vitro in medium supplemented with 10% heat inactivated fetal bovine serum at 37° C. in an atmosphere of 5% CO₂ in air. The tumor cells were routinely sub-cultured twice weekly by trypsin-EDTA treatment. The cells growing in an exponential growth phase were harvested and counted for tumor inoculation.

### 4.2. Tumor Inoculation

Each mouse was inoculated subcutaneously at the right flank with PC-3 tumor cells (10×10⁶) in 0.2 mL of PBS for tumor development. The animals were randomized and dosed when the average tumor volume reached approximately 440 mm³ for the PK/PD study. The test article administration and time points in each group were shown in the experimental design table.

### 4.3. Testing Article Formulation Preparation

| | Conc. | |
|---|---|---|
| Treatment | (mg/mL) | Formulation |
| Vehicle | — | 25 mM Histidine 10% Sucrose pH 7 |
| BT5528 | 0.15 | Dissolve 1.88 mg BT5528 in 3.71 mL Ace-buffer¹ to make |
| | | a 0.5 mg/mL stock. Dilute 3 mL 0.5 mg/mL stock with 7 |
| | | mL Ace-buffer |
| | 0.05 | Dilute 3 mL 0.15 mg/mL BT5528 with 6 mL Ace-buffer |
| | 0.0167 | Dilute 3 mL 0.05 mg/mL BT5528 with 6 mL Ace-buffer |
| Non-binding | 0.15 | Dissolve 1.93 mg Non-binding BTC in 3.71 mL His- |
| BTC | | buffer² to make a 0.5 mg/mL stock. Dilute 3 mL 0.5 |
| | | mg/mL stock with 7 mL His-buffer |
| | 0.05 | Dilute 3 mL 0.15 mg/mL Non-binding BTC with 6 mL |
| | | His-buffer |
| EphA2-ADC | 0.3 | Dilute 0.6 mL 4.24 mg/mL with 7.88 mL ADC-buffer³ |
| | 0.1 | Dilute 2.5 mL 0.3 mg/mL with 5 mL ADC-buffer |

¹Ace-buffer: 50 mM Acetate 10% Sucrose pH 5
²His-buffer: 25 mM Histidine 10% Sucrose pH 7
³ADC-buffer: 25 mM Histidine pH 5.

### 4.4. Observations

All the procedures related to animal handling, care and the treatment in the study were performed according to the guidelines, following the guidance of the Association for Assessment and Accreditation of Laboratory Animal Care (AAALAC). At the time of routine monitoring, the animals were daily checked for any effects of tumor growth and treatments on normal behavior such as mobility, food and water consumption (by looking only), body weight gain/loss (body weights were measured every day), eye/hair matting and any other abnormal effect as stated in the protocol. Death and observed clinical signs were recorded on the basis of the numbers of animals within each subset.

### 4.5. Sample Collection

Mice were randomly grouped based on tumor volume and dosed as experimental design. Plasma, serum, muscle and tumors were collected at 1 h, 2 h, 8 h, 24 h, 48 h, 72 h and 96 h post dosing.

3 tumors of a mouse cohort were embed into 1 FFPE block.

### 5. Assay Methods

### 5.1. Ventana Discovery Protocol for CC3:

5 μm FFPE tissue section loaded on Ventana XT;
Deparaffinization was selected;
EDTA based CC1 Standard heat antigen retravel was selected;
Option 1 (Protein blocker, Invitrogen Cat #1890588) was selected and incubate for 32 Min;
CC3 antibody (CST Cat #9661) 1:200 dilution was applied and incubated for 60 min at 37° C.
Apply One Drop of [OMap anti-Rb HRP] (Multimer HRP, Cat #760-4311), and Incubate for 16 Minutes;
ChromMap DAB (Cat #760-159) was applied after multimer HRP incubation.
Counterstain with HEMATOXYLIN for 8 min;
Apply One Drop of BLUING REAGENT and incubation for 4 mins;
Rinse with diluted detergent to remove LCS and tap water wash a few times;
Dehydration in an ascending series alcohol and clear in xylene 3 times;
Mounting and cover slipping
   5.2. Ventana Discovery Protocol for pHH3:
5 μm FFPE tissue section loaded on Ventana XT;
Deparaffinization was selected;
EDTA based CC1 Standard heat antigen retravel was selected;
Option 1 (Protein blocker, Invitrogen Cat #1890588) was selected and incubate for 32 Min;
pHH3 antibody (CST Cat #9701) 1:200 dilution was applied and incubated for 60 min at 37° C.
Apply One Drop of [OMap anti-Rb HRP] (Multimer HRP, Cat #760-4311), and Incubate for 16 Minutes;
ChromMap DAB (Cat #760-159) was applied after multimer HRP incubation.
Counterstain with HEMATOXYLIN for 8 min;
Apply One Drop of BLUING REAGENT and incubation for 4 mins;
Rinse with diluted detergent to remove LCS and tap water wash a few times;
Dehydration in an ascending series alcohol and clear in xylene 3 times;
Mounting and cover slipping

### 5.3. LC-MS Quantitation of MMAE in Plasma, Tumour and Muscle

LIST OF ABBREVIATIONS AND DEFINITIONS OF TERMS

| Abbreviation | Definition of Abbreviation |
|---|---|
| ° C. | Degrees Celsius |
| AEBSF | 4-(2-Aminoethyl)benzenesulfonyl fluoride |
| DIL | Dilution |
| L | Litre |
| LC | Liquid Chromatography |
| LC-MS/MS | Liquid Chromatography Coupled to Tandem |
| | Mass Spectrometry |
| LLOQ | Lower Limit of Quantitation |
| MMAE | Monomethyl Auristatin E |
| μg | Microgram |
| μL | Microlitre |
| mg | Milligram |
| mL | Millilitre |
| mm | Millimetre |
| mM | Millimolar |
| MRM | Multiple Reaction Monitoring |
| MS | Mass Spectrometer |
| ng | Nanogram |
| QC | Quality Control |
| WS | Working Solution |
| YBS | York Bioanalytical Solutions |

### REFERENCE MATERIALS

Test Compounds and Internal Standard

| | Molecular | | Purity | |
|---|---|---|---|---|
| Compound | Weight | Batch Number | (%) | Supplier |
| MMAE | 717.98 | 18841 | 98.29 | MedChem Express |
| D8 MMAE | 726.03 | HY-15162A-1M | 98.31 | MedChem Express |

### Protease Inhibitor Solutions

Two protease inhibitor products were used: Complete™ mini EDTA-free protease inhibitor cocktail tablets and AEBSF (200 mM, prepared by dissolving 100 mg in water (2086 μL)). The AEBSF solution was stored at −20° C. when not in use. For the dilution of plasma, a solution (inhibitor solution A) was prepared by dissolving 12 Complete™ tablets in 50/50 methanol/water (12 mL) and adding 200 mM AEBSF (12 μL). For the dilution and homogenisation of tissue, a solution (inhibitor solution B) was prepared by dissolving 70 Complete™ tablets in 50/50 methanol/water (700 mL) and adding 200 mM AEBSF (700 μL). Both solutions were stored at 4° C. and kept on ice when in use.

### Control Matrices and Blank Matrix Preparation

Control mouse matrices (tumour and muscle) were received with the study samples from Wuxi, Shanghai, China (study YEA/007). Additional plasma was supplied by Charles River, UK. All control matrices were stored at −80° C.

Control matrices were diluted with chilled inhibitor solution to make blank matrix. Plasma was diluted 1:1 (v/v) with inhibitor solution A, tumour and muscle were diluted 1:9 (w/v) with inhibitor solution B.

With the exception of plasma, all matrices were homogenized with a Precellys Evolution tissue homogeniser, using a cryolys filled with dry ice or liquid nitrogen. Metal MK28 beads (n=4) were added to the diluted tissue in re-inforced 2 mL tubes and the mixture was homogenized at 8500 rpm for 20 seconds, followed by a resting period to maintain the temperature. This process was repeated over 4 cycles. The resulting control matrix was used to prepare standards, QCs and blanks and for sample dilution.

### Stock Solutions

Stock solutions of MMAE and D8-MMAE were made at 1 mM and 1.5 mM respectively in DMSO (accounting for purity).

### Internal Standard Working Solutions

Internal standard working solutions were prepared in 50/50 methanol/water at 100 and 10 μmol/mL for the analysis of plasma and tissue respectively.

### Quantitative Assay Methodology

### Study Sample Preparation

Study samples were diluted as described for the preparation of blank matrix. Plasma samples were thawed on ice and an accurate volume taken for dilution with chilled inhibitor solution. Chilled inhibitor solution was added to tissue samples using the weights provided. Tissue samples were homogenized following the same methodology as used for the blank matrix preparation.

### Standards and Quality Controls

The MMAE stock solution was diluted directly into blank matrix for the preparation of standards and QCs. For each matrix, QCs were prepared at the low (S2), mid (S7) and high (S8) levels. Dilution QCs were prepared by diluting WS1 10-fold in the relevant blank matrix.

Standards and QC Preparation for the Analysis
of Plasma Samples

| | | | | Volume |
|---|---|---|---|---|
| | | Volume | Standard | Blank |
| | Standard | to Add | to Use | Matrix |
| ID | pmol/mL | μL | pmol/mL | μL |
| S1 | 0.5 | 10 | 5 | 90 |
| S2 | 1 | 20 | 5 | 80 |
| S3 | 5 | 10 | 50 | 90 |
| S4 | 50 | 10 | 500 | 90 |
| S5 | 150 | 3 | 5000 | 97 |
| S6 | 400 | 8 | 5000 | 92 |
| S7 | 500 | 10 | 5000 | 90 |
| S8 | 900 | 18 | 5000 | 82 |
| S9 | 1000 | 20 | 5000 | 80 |
| WS1 | 5000 | 2.5 | Stock solution | 497.5 |

Standards and QC Preparation for the Analysis
of Tissue Samples

| | | | | Volume |
|---|---|---|---|---|
| | | Volume | Standard | Blank |
| | Standard | to Add | to Use | Matrix |
| | pmol/mL | μL | pmol/mL | μL |
| S1 | 0.1 | 10 | 1 | 90 |
| S2 | 0.2 | 20 | 1 | 80 |
| S3 | 1 | 10 | 10 | 90 |
| S4 | 10 | 10 | 100 | 90 |
| S5 | 30 | 3 | 1000 | 97 |
| S6 | 80 | 8 | 1000 | 92 |
| S7 | 100 | 10 | 1000 | 90 |
| S8 | 180 | 18 | 1000 | 82 |
| S9 | 200 | 20 | 1000 | 80 |
| WS1 | 1000 | 25 | 5000 pmol/mL | 100 |
| | | | plasma WS1 | |

### Sample Extraction

Samples, standards, QCs and blanks were transferred (10 μL diluted plasma, 30 μL tissue homogenate) to a 96-well plate on ice. Dilution QCs were prepared by diluting 5 μL WS1 into 45 μL blank matrix. During sample analysis, 6 samples were selected to re-run diluted (to check for any matrix effects by comparing to the neat sample) and were prepared in the same way. After mixing, samples were transferred (10 μL diluted plasma, 30 μL tissue homogenate) to the plate. Internal standard working solution was added (10 μL) to all samples except the double blank. Cold methanol was added to all wells (300 μL for plasma, 200 μL for all other matrices) and the plate was mixed on a plate shaker and then centrifuged (1780 g at 4° C. for 10 minutes). All Standards, QCs, samples and blanks were diluted into a fresh 96-well plate containing 50/50 methanol/water. For the plasma analysis, 100 μL was diluted with 400 μL; for the tissue analysis, 100 μL was diluted with 100 μL. The plate was mixed briefly and sealed ready for injection onto the LC-MS/MS.

### LC-MS/MS Conditions

Samples were injected onto a LC-MS/MS system, which consisted of an API5000 mass spectrometer (Sciex), a 1290 pump (Agilent) and an HTS Pal auto-sampler (CTC analytics).

MRM Parameters

| Analyte | Q1 Mass | Q3 Mass | CE | DP |
|---|---|---|---|---|
| MMAE | 718.7 | 686.7 | 37 | 120 |
| D8-MMAE | 726.6 | 694.6 | 37 | 90 |

MS Conditions

| Polarity | CAD | CUR | GS1 | GS2 | IS | TEM | EP | CXP |
|---|---|---|---|---|---|---|---|---|
| Positive | 8 | 30 | 50 | 50 | 4500 | 700 | 10 | 15 |

### LC Parameters

Pump: Agilent 1290 binary pump

### Analytical Column: 2.1×100 mm Acquity CSH C18, 1.7 μm (Waters)

Mobile Phase A: 0.1% formic acid in water
Mobile Phase B: 0.1% formic acid in acetonitrile
Wash Solvents: Water containing 1% acetic acid
40/30/30 methanol/isopropylalcohol/acetone

### Injection Volume: 30 μL

### LC Gradient:

| Time | Flow Rate | A | B |
|---|---|---|---|
| (min) | (μL/min) | (%) | (%) |
| 0.0 | 600 | 75 | 25 |
| 0.2 | 600 | 75 | 25 |
| 2.0 | 600 | 50 | 50 |
| 2.1 | 600 | 10 | 90 |
| 2.8 | 600 | 10 | 90 |
| 2.9 | 600 | 75 | 25 |
| 4.0 | 600 | 75 | 25 |

### 6. Results

### 6.1. BT5528 Delivers MMAE to Tumor

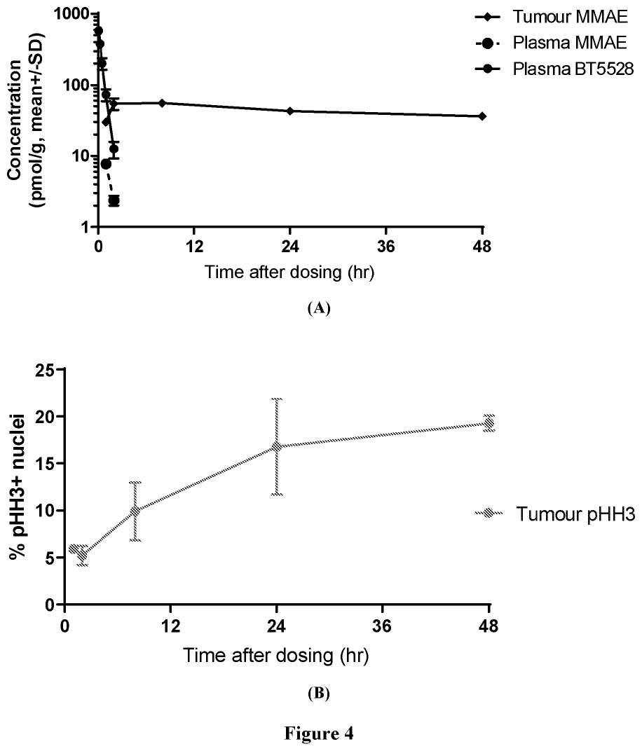

The concentrations of tumor MMAE, plasma MMAE, and plasma BT5528 after a single dose of BT5528 are shown in FIG. 4(A). A single dose of BT5528 is shown to produce high MMAE concentrations in tumour, which is stable from 2h to >48h, and to result a transient exposure of both BT5528 and MMAE in plasma.

### 6.2. BT5528 Induces Mitotic Arrest in Tumor

Tumor pHH3 after a single dose of BT5528 is shown in FIG. 4(B). A single dose of BT5528 is shown to induce mitotic arrest in tumor, which is measurable by pHH3 IHC within 24 hours.

### Example 5. In Vivo of Efficacy Study of Test Articles in Treatment of Pancreatic Ductal Adenocarcinoma (PDAC) in PDX Models

BT5528 and Vehicle were prepared as described in the examples above, and tested in treatment of Pancreatic Ductal Adenocarcinoma (PDAC) in PDX models. PDX models effectively capture patient responses to oncology therapy in a heterogeneous cohort of patients with solid tumors with 80-100% correlation between the PDX and patient response (Izumchenko et al. 2017)

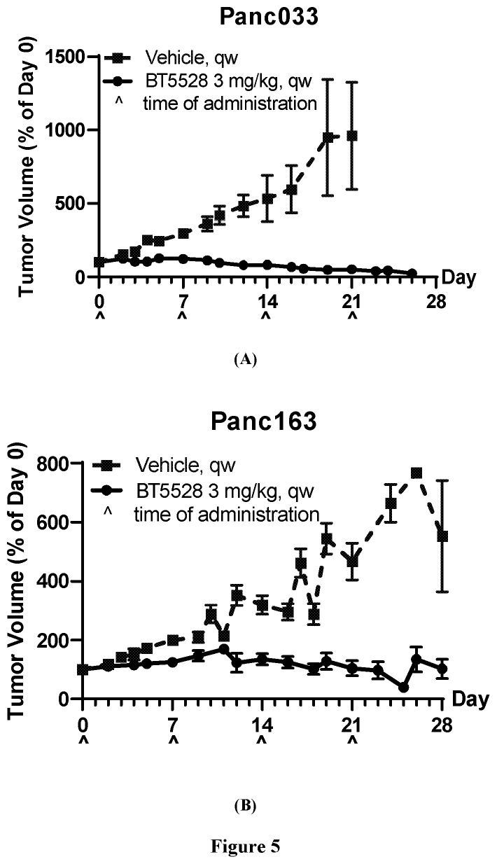

Pancreatic ductal adenocarcinoma patient derived xenograft tumors (PDAC PDX; Panc033 and Panc163) were implanted subcutaneously from source tumors into the flank of NSG mice. Tumor bearing animals were randomized to receive intravenously a weekly dosing of vehicle or 3 mg/kg BT5528. Tumor sizes were monitored by caliper measurements. BT5528 treatment demonstrated significant anti-tumor activity from reduced tumor growth rate to decreasing tumor volumes over 4-week treatment period. The tumor volume traces after the treatment is shown in FIG. 5.

### Example 6. Efficacy Evaluation of BT5528 Against Established Intracardially Implanted PC-3M-Luc-C6 Human Prostate Carcinoma in Male Nude Mice

The purpose of this study was to evaluate the efficacy and overall impact of Bicycle toxin conjugate BT5528 against established intracardially implanted PC-3M-luc-C6 human prostate carcinoma in male nude mice, with treatment regimens starting at different disease stages (Day 14 and Day 21). Response was monitored using bioluminescence imaging (BLI) on Days 14, 21, 28, 35, and 42 coupled with traditional survival endpoints.

All treatments were well tolerated with body weight loss associated with advancing disease and no treatment-related deaths. Group 3 mice had treatment initiated a week later than Groups 1 and 2 (by study design), which affected lifespan measurements.

Treatment with BT5528, starting Day 14, resulted in an increased lifespan (ILS) of 177.3%, a tumor growth delay of >35.1 days, a Day 26 T/C of 1%, and a 60% incidence of partial regressions. Group 2 Mouse 2 had no remarkable necropsy findings at study end on Day 78.

Treatment with BT5528, starting Day 21, resulted in nearly identical levels of activity with an increased lifespan (ILS) of 145.5%, a tumor growth delay of >35.1 days, a Day 26 T/C of 3.4%. However, starting treatment one week later resulted in 40% less incidence of partial regressions.

### 1. Materials

### 1.1. Test Agents and Vehicles

Vehicle: 50 mM Acetate, 10% Sucrose:
Storage: −80° C.
Formulation pH: 5
Dose volume: 0.01 mL/g

BT5528:
Formulation pH: 5.4
High Dose Formulation: 0.15 mg/mL
Vehicle: 50 mM Acetate, 10% Sucrose
Storage: 4° C.
Dose volume: 0.01 mL/g

### 1.2. Animals

All procedures carried out in this experiment were conducted in compliance with the applicable laws, regulations and guidelines of the National Institutes of Health (NIH).
Species: Mouse
Strain: Envigo nude mice (Hsd:Athymic Nude-Foxn1^{nu})
Age at implant: 5-6 weeks
Sex: male
Min weight (D14): 24.9 g
Mean weight (D14): 28.4 g (range of group means, 27.7-28.8 g)

The mice were kept in innovive disposable ventilated cages with corn cob bedding inside Biobubble clean rooms, with 3 animals in each cage.
Temperature: 70±2° F.
Humidity 30-70%.
Diet: Teklad 2918.15 Rodent Diet.
Water: Ad libitum.
Acclimation: 3 days
Animal identification: Animals were marked by ear punch.

### 1.3. Cell Preparation/Implantation

| Model | PC-3M-Luc-C6 | Histotype | Human Prostate |
|---|---|---|---|
| | | | Adenocarcinoma |
| Source | PE (Xenogen) (Caliper) | Implant type | cells |
| Media | Modified Eagle Medium | Dissociation | 0.25% trypsin/2.21 mM |
| | (MEM) supplemented with 1 | solution | EDTA in HBSS |
| | mM Na pyruvate, 1% NEAA, | | |
| | 2 mM L-glutamine, 1% MEM | | |
| | vitamins and modified with | | |
| | 10% NHI FBS + 1% PSG | | |
| Route | Intracardiac | Location | Left ventricular space |
| Inoculum | 3.0E+06 trypan-excluding cells | Implant media | Dulbecco's Phosphate |
| | | | Buffered Saline (DPBS) |
| Matrigel | 0% | Inj. Volume | 0.1 mL |
| Viability (pre) | 95% | Viability (post) | 88% |

Mice were anesthetized for implant.

### 1.4. Intracardiac Implantation

Animals were anesthetized with an IP injection of a ketamine (100 mg/kg)/xylazine (6 mg/kg) cocktail. When the animals were non-responsive (determined by a toe pinch test), 100 μl of cell suspension (3.0 E+06 cells) were drawn into a 1 ml syringe and a 27×½″ gauge needle was then attached. A small air bubble is created in the plunger side of the syringe before injection. The needle was then inserted slowly through the center of the second intercostal space, approximately 3 mm to the left of the sternum and aimed centrally until a continuous pulsation of bright red oxygenated blood into the needle hub was observed. 100 μl of cell suspension was then slowly injected over 5 seconds. A fresh needle and syringe were used for each animal.

Animals were injected with firefly D-luciferin (150 mg/kg) by IP administration according to body weight (0.2 ml/20 g). The success of the intracardiac injections was verified by a one minute bioluminescence scan using large binning (high sensitivity) of the CCD chip immediately after the injection. Mice with signals from the entire body, from snout to base of the tail, were deemed to have been successfully injected, while mice with signals localized to the chest area only, or not fully extending into the snout, were triaged from the study immediately.

Mice were maintained throughout the procedure on a heated water blanket. Successfully injected mice were allowed to recover from anesthetic and monitored until fully awake and able to walk.

### 2. Treatment

All mice were sorted into study groups based on body weight and BLI-derived estimation of tumor burden.

| Group | N | Treatment | Dose | ROA | Regimen | Days of treatment |
|---|---|---|---|---|---|---|
| 1 | 5 | Vehicle | 0.2 mL/ | IV | Q7D × 4; | Days 14, 21, |
| | | Control | 120 g | | D14 | 28 and 35 |
| 2 | 5 | BT5528 | 150 mg/kg | IV | Q7D × 4; | Days 14, 21, |
| | | | | | D14 | 28 and 35 |
| 3 | 5 | BT5528 | 150 mg/kg | IV | Q7D × 4; | Days 21, 28, |
| | | | | | D21 | 35 and 42 |

### 3. Sampling

| Group(s) | Animals | Tissue (s) | Time Pts | Product | Description |
|---|---|---|---|---|---|
| 1 | 3 | Tumor | End of life | Tumor | Tumor nodules from the thoracic |
| | | nodules | (D36) | nodules in | cavity were collected and placed in |
| | | | | 10% NBF | 10% NBF and transferred to the |
| | | | | | histologist for formalin fixed |
| | | | | | paraffin embedding (FFPE) into |
| | | | | | blocks |

### 4. Imaging

| Group(s) | Animals | Modality | Imaging days | Output | Comments |
|---|---|---|---|---|---|
| 1 | All | BLI | Days 0, 7, 14, | Total flux: | The total tumor burden (total |
| | | | 21, 28 and 35 | photons/second | bone signal) of the animals |
| | | | | | was calculated by the |
| | | | | | summation of signal from ROIs |
| | | | | | placed over the left hind limb, |
| | | | | | right hind limb and the |
| | | | | | mandible, in both the prone |
| | | | | | and supine positions, |
| 2, 3 | All | BLI | Days 0, 7, 14, | Total flux: | The total tumor burden (total |
| | | | 21, 28, 35, | photons/second | bone signal) of the animals |
| | | | 42, 49, 56 | | was calculated by the |
| | | | and 63 | | summation of signal from ROIs |
| | | | | | placed over the left hind limb, |
| | | | | | right hind limb and the |
| | | | | | mandible, in both the prone |
| | | | | | and supine positions. |

BLI imaging on Day 0 was immediately after cell injections to determine if the injections were successful. BLI imaging on Day 7 was for all animals prior to study enrollment to assess disease progression. Group 1 animals' last imaging time point was Day 35 and they were all deceased by Day 41.

5. In vivo Bioluminescence Imaging (BLI)

Bioluminescence refers to light produced by the enzymatic reaction of a luciferase enzyme with its substrate. Bioluminescence imaging (BLI) of luciferase-expressing tumor cell lines enables a noninvasive determination of site-localized tumor burden. The quantity of emitted light from the tumor after systemic injection of D-luciferin is expected to correlate with viable tumor burden.

D-Luciferin (lot #0000307215) was obtained from Promega as a white powder and stored at −80° C. in a covered box to minimize light exposure. Saline was added to the D-luciferin powder to produce a clear yellow solution. A 15 mg/ml solution was prepared for in vivo imaging. D-Luciferin was prepared immediately prior to each bioluminescence imaging session and stored protected from light on wet ice during use.

BLI was performed using an IVIS S5 Lumina system (PerkinElmer, Waltham, Mass.). Animals were imaged five at a time under 1-2% isoflurane gas anesthesia. Each mouse was injected IP with 150 mg/kg (15 mg/ml) D-luciferin and imaged in the prone then supine positions 10 minutes after the injection. Large binning of the CCD chip was used, and the exposure time was adjusted (10 seconds to 2 minutes) to obtain at least several hundred counts per image and to avoid saturation of the CCD chip. BLI images were collected on Days 0, 7, 14, 21, 28, 35, 42, 49, 56 and 63 post-implant.

Images were analyzed using Living Image 4.7.1 (PerkinElmer, MA) software. Each unique tissue signal was circled manually and labeled based on anatomical site as mandible or hind limb for both prone and supine images. For limbs, the signal was also designated as being from the right or left side of the mouse.

Signal flux (photons/sec) were calculated for each unique metastatic signal and exported for all ROIs to facilitate analyses between groups.

### 6. Pharmacology and Imaging Endpoints

| | | Primary | Primary | Primary | Other |
|---|---|---|---|---|---|
| Data Type | Method | Endpoint 1 | Endpoint 2 | Endpoint 3 | Endpoints |
| Pharmacology | Body | Increase in | Treatment- | Treatment- | |
| | weights, | Lifespan (ILS) | related weight | related deaths | |
| | clinical | (%) | change (%) | (%) | |
| | observations | | | | |
| Imaging | BLI | Tumor growth | % T/C (Day 26) | Regressions | Tumor doubling |
| | | delay (Evaluation | | (PR, CR, TFS) | time (days) |
| | | size of | | | |
| | | 9.0E+07 p/s) | | | |

Study was terminated on Day 78. All BLI endpoint calculations were derived from total bone values. The BLI background signal for this study was measured at 1.20 E+05 p/s. An animal was credited with a partial regression if the BLI signal fell below half the staging BLI signal level. Similarly, a complete regression (CR) was credited if the BLI signal fell below the background level, and a tumor-free survivor was if the BLI signal was below background on last day of imaging (Day 63), with no evidence of disease at necropsy.

### 7. Results

The mean estimated tumor burden for all groups in the experiment on the first day of treatment (for G1 and G2) was 7.53 E+06p/s and all of the groups in the experiment were well-matched (range of group means, 7.29 E+06-7.68 E+06p/s). All animals weighed at least 24.9 g at the initiation of therapy. Mean group body weights at first treatment were also well matched (range of group means, 27.7-28.8 g). BLI background signal for this study was measured at 1.20 E+05 p/s for this study. A tumor burden of 9.00 E+07 p/s was chosen for evaluation of efficacy by tumor growth delay. In the Control Group, the median time to evaluation size was 27.9 days, and the median tumor volume doubling time was 3.3 days. Control animals experienced 17.1% mean weight loss during the treatment regimen, likely due to disease progression. The median lifespan in the Control Group was 22 days. There were no spontaneous regressions in the Control Group. 3 of 3 thioglycolate cultures of cells used for implantation of this study were negative for gross bacterial contamination.

All of this information is consistent with historical norms and the experiment was judged to be technically satisfactory and the data appropriate for evaluation.

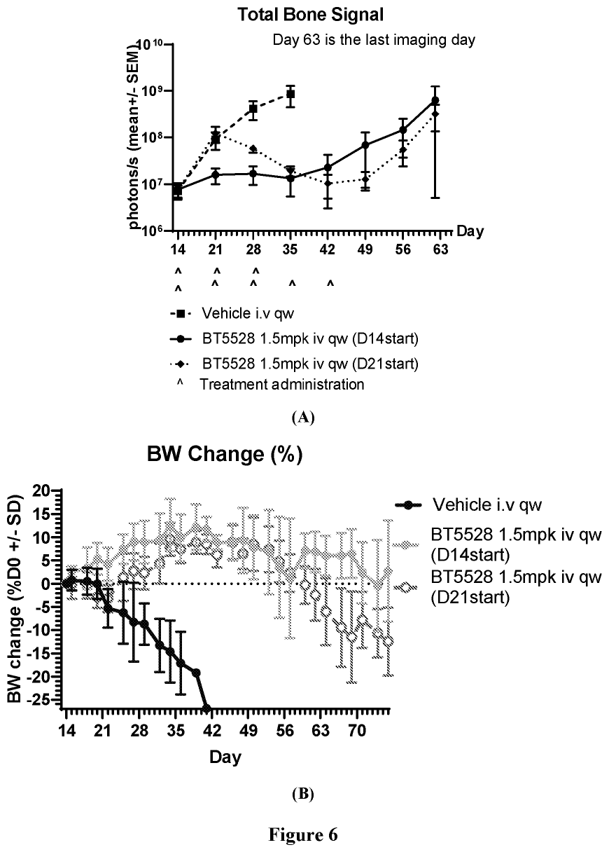

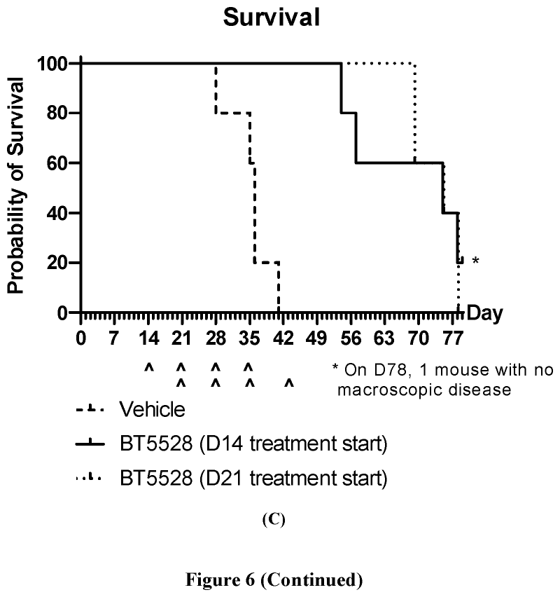

BT5528 demonstrates activity against metastatic disease: reduction of tumor cell burden in bone lesions. The total bone signal, BW change (%), and percentage survival of the mice after the vehicle and BT5528 treatment are shown in FIG. 6(A)-(C). It was found that:
PC3 metastatic lesions have the required enzymatic activity for payload release from BT5528 to yield significant anti-tumor activity;
˜16 fold difference in total cell burden (back calculated from in vitro data for photons/s per cell) in bone between D14 and D21 treatment initiation, BT5528 active in both settings
4 weekly BT5528 treatment cycles reduced the bone tumor cell burden significantly and extended the survival of the mice
1 mouse at the end of the study (D78) without observable (macroscopic) disease.

### 8. Definition of Terms

Day 0—The day on which the tumors are implanted.

Treatment related deaths (%)—An animal is presumed to experience a treatment-related death if it is found dead or is euthanized in moribund condition during or within two weeks after the last treatment with a tumor burden less than half that of the smallest lethal tumor in the control group, and only if the animal shows no evidence of infection, mechanical dosing trauma, or other obvious causes of morbidity at necropsy. Animals euthanized during the same period for other causes (sampling, accidental trauma, etc.) are excluded from this calculation. This designation is meant to help identify animals that may have experienced drug induced toxicity, but it does not directly imply causality. (Group toxicity parameter)

Treatment-related weight change—This is a group endpoint calculated from the group mean body weights. It is calculated differently for specific circumstances as follows:
If (at any point between the first day of treatment and two weeks after the final treatment) the mean group body weight decreases by more than 2%, the maximum weight loss is reported, even if body weight eventually rebounded during treatment to a net weight gain. In the special case of a rebound to a net gain, the recovery is thoroughly noted in the results section.
If mean group body weights do not decrease by more than 2% at any point, the body weight change is reported as the difference between the body weight on the first day of treatment and the date that is two weeks after the end of treatment.
The duration of treatment can vary by group, so direct comparison of weight gains (in particular) needs to account for that.
When weight loss occurs, in models that typically have tumor progression induced weight loss, multiple factors influence body weight change. To assess the contribution of test agents to weight loss, Net Treatment Related Weight Loss may be used. This is done in two different ways depending on the degree of efficacy observed in the study.
   When no efficacy is apparent, Net weight loss is calculated by subtracting the mean weight loss in the control group from the mean weight loss in the treated group for every day of the study.
   When efficacy is observed, widely differing tumor burdens can occur between the control and treated groups. When this occurs, net weight loss is calculated by normalizing for tumor burden. We do this by constructing a plot of control group mean tumor burden vs control group mean weight loss, using all of the weight data available for the control group. (Typically log/linear plots of tumor burden vs weight loss are easiest to use.) On any given study day, the net weight loss of the treated group is estimated by looking up the mean tumor burden of the treated group on the control group reference plot and reading off the implied/expected weight loss due to tumor burden. This value is then subtracted from the mean weight loss in the treated group to generate the net weight loss for the treated group on that day. The calculated net weight loss is then used to estimate the tolerance to the drug.
   Median T/C—Is a group endpoint. It is calculated for each day of treatment as:

$$Median \frac{T}{C} = ( \frac{median ( {T}_{t} )}{median ( {C}_{t} )} ) * 100$$

Time to Evaluation Size (TES)—TES is an individual mouse endpoint and it is expressed in days from tumor implant. It is the time it takes the tumor burden to reach a specified value, and it can be calculated from any method of evaluating tumor burden (caliper measurements, BLI, anatomical imaging, etc.). It is calculated by log-linear interpolation between the two closest data points that bracket the chosen tumor burden.

$${D}_{ES} = {D}_{h} - \frac{( ( log {V}_{h} - log ES ) * ( {D}_{h} - {D}_{l} ) )}{( log {V}_{h} - log {V}_{l} )}$$

where:

D_{ES}=TESᵢ—the day evaluation size is reached

Dₕ—the day of the first measurement greater than the ES was reached

Dₗ—the day of the last measurement before the ES was reached

Vₕ—The tumor volume on day Dₕ

Vₗ—the tumor volume on Dₗ

ES—the evaluation size

Tumor doubling time (Td)—Td is an individual and group parameter, typically expressed as the median Td of the group. It is measured in days. Td can be calculated from any type of volumetric data (caliper measurements, BLI signals, etc). For QC purposes it is calculated for the exponential portion of the tumor growth curve. Data points during any lag phase and in the Gompertzian advanced stage are not included. Typical tumor burden limits are between 100 and 1000 mm3, but actual selection is data driven. Td is calculated for each mouse from a least squares best fit of a log/linear plot of tumor burden vs day as:

Td=log 2/slope

On rare occasions the median Td is used as a potential indicator of efficacy. As such it is calculated as the median for every group, over a specified range of days thought to reflect a period of response to therapy.

Tumor growth delay (TGD, or T-C)—TGD is a group endpoint. Tumor growth delay is expressed in units of days and is calculated from the median times it takes the mice in a group to reach a specified tumor burden (time to evaluation size, TES). It can be calculated as:

TGD=median TES treated−median TES control

Tumor Regressions
Complete Regression (CR)—an animal is credited a complete regression if its tumor burden decreases to less than a declared background BLI signal level.
Partial regression—An animal is credited with a partial regression if its tumor burden decreases to less than half of the tumor burden at first treatment. The PR must be maintained for at least 2 consecutive measurements for caliper driven studies. (For BLI driven studies the required confirmation is waived because of the dynamic range of the measurements and typically longer intervals between imaging.) PRs are tabulated exclusive of CRs, thus an animal that achieves a CR is not also counted as a PR. (Individual efficacy parameter)

Tumor-free Survivor (TFS)—A TFS is any animal that (1) survives until termination of the study, and (2) has no reliably measurable evidence of disease at study termination. Mice that are tumor-free at some point during the study, but are then euthanized for sampling or other purposes prior to the end of the study are not considered TFS. They are excluded from calculation of the % TFS. TFS status does not imply “cure.”

Median Lifespan—Lifespan is an individual mouse endpoint. It is measured from the day of first treatment in the study (not the day of tumor implant) for each animal. It captures the day of death for all animals that either die or are euthanized for disease or treatment related causes. Animals euthanized for sampling or other causes unrelated to disease or therapy are excluded from this calculation. The median lifespan for the group is used to calculate the % Increase in lifespan (% ILS). When animals are euthanized for IACUC mandated maximum tumor burdens, Time to Progression (TP) is used instead of this variable.

% Increase in lifespan (ILS)−% ILS is a group endpoint. It is calculated as

$$\% ILS = \{ \frac{\begin{matrix}[ ( median Treated Lifespan ) - \\ ( median Control Lifespan ) ]\end{matrix}}{median Control Lifespan} \} * 100$$

### Example 7. In Vivo Evaluation in Low Passage Champions TumorGraft® Models of Human Non-Small Cell Lung Cancer in Immunocompromised Mice in Champions PDX Indication Screen

### List of Abbreviations

### CFR Code of Federal Regulations

### Champions Champions Oncology, Inc.

CR complete responder

### FDA Food and Drug Administration

### GLP Good Laboratory Practice

IV intravenous
L length
MTD maximum tolerated dose
MTV mean tumor volume
NSCLC non-small cell lung cancer
PO oral gavage
Q7D once every 7 days
SC subcutaneous(ly)
SEM standard error of the mean
TV tumor volume
W width

### Summary

This study was conducted to evaluate the in vivo antitumor activity of BT5528 as monotherapy in 15 low-passage Champions TumorGraft® models.

Mice were implanted subcutaneously into the left flank with tumor fragments from one of the various models. After tumors grew to an average of 150-300 mm³, mice were treated by IV administration Q7Dx4 (n=2) with vehicle or 3 mg/kg of BT5528. Effects on tumor growth were evaluated by measuring percent tumor growth inhibition (% TGI), and the number of complete responders (CR), partial responders (PR), and tumor-free survivors (TFS). Tolerability was assessed by body weight loss, lethality, and clinical signs of adverse treatment-related side effects. Tumor volumes and body weights were measured twice a week.

Weekly 3 mpk BT5528 treatment yields to a varied range of anti-tumor activity in a panel of 15 NSCLC patient derived tumor xenografts (FIG. 8). Notably 10 out of 15 models shows tumor growth inhibition of 50% or more. There was no PR, CR or TFS in any group. All treatments were tolerated in all models except one animal was found dead in model CTG-0170.

### 1. Objectives

The objective of this study was to determine the in vivo antitumor activity of BT5528 as monotherapy in 15 low-passage Champions TumorGraft® models CTG-0160, CTG-0170, CTG-0178, CTG-0192, CTG-0363, CTG-0808, CTG-0838, CTG-0848, CTG-1212, CTG-1502, CTG-1535, CTG-2011, CTG-2393, CTG-2539 and CTG-2540, representing human non-small cell lung cancer in immunocompromised mice.

### 2. Materials and Methods

### 2.1 Tumor Models

| Model | Tumor | Passage |
|---|---|---|
| Number | Type | Number |
| CTG-0160 | NSCLC | 6 |
| CTG-0170 | NSCLC | 5 |
| CTG-0178 | NSCLC | 7 |
| CTG-0192 | NSCLC | 6 |
| CTG-0363 | NSCLC | 8 |
| CTG-0808 | NSCLC | 5 |
| CTG-0838 | NSCLC | 7 |
| CTG-0848 | NSCLC | 7 |
| CTG-1212 | NSCLC | 8 |
| CTG-1502 | NSCLC | 5 |
| CTG-1535 | NSCLC | 8 |
| CTG-2011 | NSCLC | 5 |
| CTG-2393 | NSCLC | 6 |
| CTG-2539 | NSCLC | 8 |
| CTG-2540 | NSCLC | 7 |

### 2.2 Test and Control Articles

Dosing solutions of BT5528 0.3 mg/kg were pre-formulated. These dosing solutions were stored at −80° C. in the dark. On each day of dosing, a frozen aliquot of each test agent was thawed, stored at 2-8° C. and used for dosing.

The vehicle, 25 mM histidine and 10% sucrose, pH 7 was stored at −80° C. in the dark. On each day of dosing, an frozen aliquot was thawed and used for dosing.

All test agents and vehicle were stable for 1 year from the date of formulation when stored at −80° C. and were sufficiently stable for the duration of this study.

### 2.3 Study Animals

The animals used in this study are described below:
Species: Mus musculus
Strain: Athymic Nude-Foxn1^{nu} (immune-compromised)
Source: Envigo (Indianapolis, Ind., USA)
Number of animals per group: 2
Age and sex: At least 6-8 weeks of age at start of dosing, female
Weight: At least 18 grams at start of dosing
Acclimation period: 3 days

### 2.4 Animal Housing and Welfare

Immunocompromised female mice between 5-8 weeks of age were housed on irradiated corncob bedding (Teklad 7902, CS) and 100% virgin kraft nesting sheets (Innorichment™) in individual HEPA ventilated cages (Innocage® IVC, Innovive USA) on a 14-10-hour light-dark cycle at 68-74° F. (20-23° C.) and 30-70% humidity. Animals had access to water (reverse osmosis, 2 ppm C12) and an irradiated test rodent diet (Teklad 2919; 19% protein, 9% fat, and 4% fiber) ad libitum. Animals exhibiting ≥10% weight loss when compared to Day 0 were provided with DietGel™ (ClearH₂O®, Westbrook, Me.) ad libitum.

All experimental procedures were performed according to the guidelines of the Institutional Animal Care and Use Committee (IACUC).

### 2.5 Experimental Design

Stock mice were implanted with tumor cells from Champions TumorGraft® models CTG-0160, CTG-0170, CTG-0178, CTG-0192, CTG-0363, CTG-0808, CTG-0838, CTG-0848, CTG-1212, CTG-1502, CTG-1535, CTG-2011, CTG-2393, CTG-2539 or CTG-2540. After the tumors reached 1000-1500 mm³, they were harvested and the tumor fragments (approximately 5×5×5 mm³) were implanted SC in the left flank of the female study mice. Each animal was implanted with a specific passage lot and documented. Tumor growth was monitored twice a week using digital calipers and the tumor volume (TV) was calculated using the formula (0.52×[length×width²]). When the TV reached an average volume of 150-300 mm³, animals were matched by tumor size and assigned into Vehicle Control and treatment groups (n=2/group) and dosing was initiated on Day 0. After the initiation of dosing on Day 0, animals were weighed twice per week using a digital scale and TV was measured twice per week and also on the final day of study. The study was completed when the mean tumor volume of Vehicle Control reached 1500 mm³ or up to Day 60, whichever occurred first. The design of the study is summarized in Table 2.

TABLE 7-1
Design of Efficacy Study in NSCLC Models

| | | | | | | | Maximum |
|---|---|---|---|---|---|---|---|
| | | | | Dose | | | Total |
| | | | Dose | Volume | | Dosing | Number |
| Group | -n- | Agent | (mg/kg) | (mL/kg) | Route | Schedule* | of Doses |
| 1 | 2 | Vehicle | 0 | 10 | IV | Q7D × 4 | 4 |
| 2 | 2 | BT5528 | 3 | 10 | IV | Q7D × 4 | 4 |

*Dosing for models CTG-0192 and CTG-1535 were extended to Q7D × 5

### 2.6 Sample Collection

Blood Collection: At study completion (7 days post final dose which was Day 28 for most models), as much blood as possible was collected from all animals in each group by cardiac puncture (under isoflurane-induced anesthesia), transferred to K₂EDTA containing tubes and mixed by gentle inversion 8-10 times. Blood samples were kept on wet ice and centrifuged as soon as practical at 3500 rpm for 10 minutes at 2-4° C. The resultant plasma was collected and transferred to uniquely labelled clear polypropylene tubes and stored at −80° C. until shipment.

Tumor Collection: At study completion (7 days post final dose which was Day 28 for most models), tumors were collected from all animals in each group and bisected: half was flash frozen, placed on dry ice and stored at −80° C. until shipment; the other half was fixed in neutral buffered formalin for 18-24 hours, transferred to 70% ethanol at room temperature for 1-3 days and sent to be paraffin embedded. Tumors that were <250 mm³ were processed as a single flash frozen sample.

### 2.7 General Toxicity

Beginning on Day 0, animals were observed daily and weighed twice weekly using a digital scale; data including individual and mean gram weights, mean percent weight change versus Day 0 (% vD₀) were recorded for each group and % vD₀ plotted at study completion. Any animal exhibiting >20% net weight loss for a period lasting 7 days or displayed >30% net body weight loss when compared to Day 0 was considered moribund and euthanized. Treatment resulting in a mean % vD₀>20% and/or >10% mortality was considered above the maximum tolerated dose. Maximum mean % vD₀ (weight nadir) for each treatment group was reported at study completion.

### 2.8 Anti-Tumor Efficacy

Inhibition of tumor growth was determined by calculating the percent TGI (100%×[1−(final MTV−initial MTV of a treated group)/(final MTV−initial MTV of the control group)]). Treatment started on Day 0.

Additional endpoints used to evaluate efficacy were: the number of complete responders (CR), partial responders (PR), and tumor-free survivors (TFS). PRs were considered exclusive of CRs, as were TFS.

| Classification | Criteria |
|---|---|
| Partial responder (PR) | TV ≤30% of TV at Day 0 for 2 consecutive measurements |
| Complete responder (CR) | TV undetectable for 2 consecutive measurements |
| Tumor-free survivor (TFS) | A CR that persists until study completion |

### 2.9 Statistical Analysis

Statistical analyses of anti-tumor efficacy were performed using one-way ANOVA followed by Dunnett's multiple comparisons test (GraphPad Prism version 8.2.0). Significant p-values=<0.05.

### 3. Amendments and Deviations

There were 1 amendment and 1 deviation in this study.

Amendment 1: blood collection from all animals at study completion (7 days post final dose). Blood will be collected by cardiac puncture and placed into K₂EDTA containing tubes and centrifuged at 3500 rpm for 10 minutes at 2-4° C. Plasma samples were collected and stored at −80° C. until shipment.

Deviation 1: Plasma samples were not collected for Groups 1, 3 and 4 for CTG-2539 as required per amendment 1. Only tumor samples were collected for these groups per protocol for CTG-2539.

### 4. Results and Discussion

### 4.1 Efficacy Study in Non-Small Cell Lung Cancer Model CTG-0160

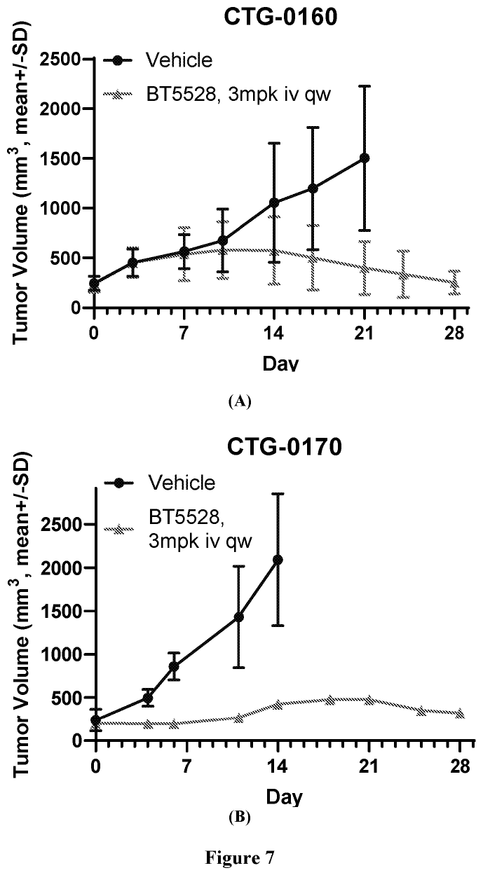

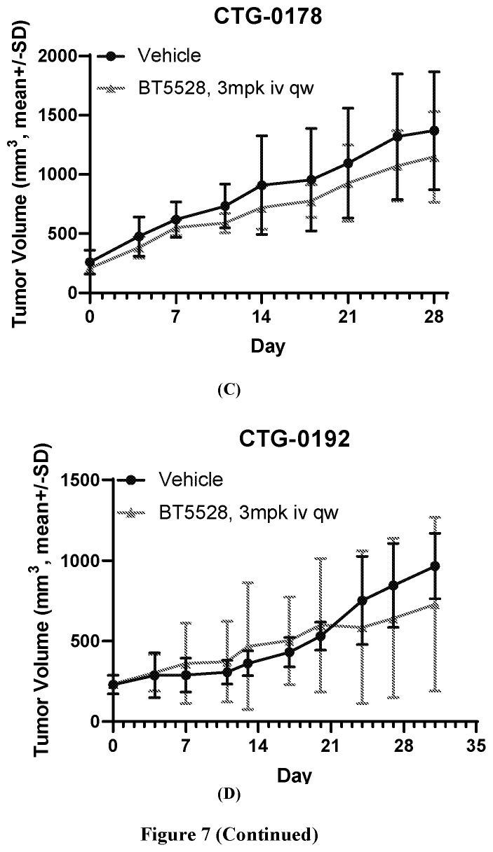

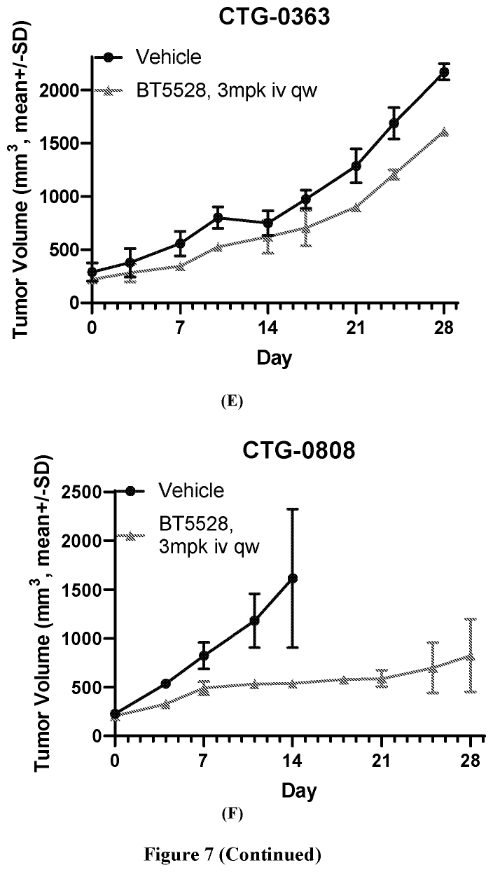

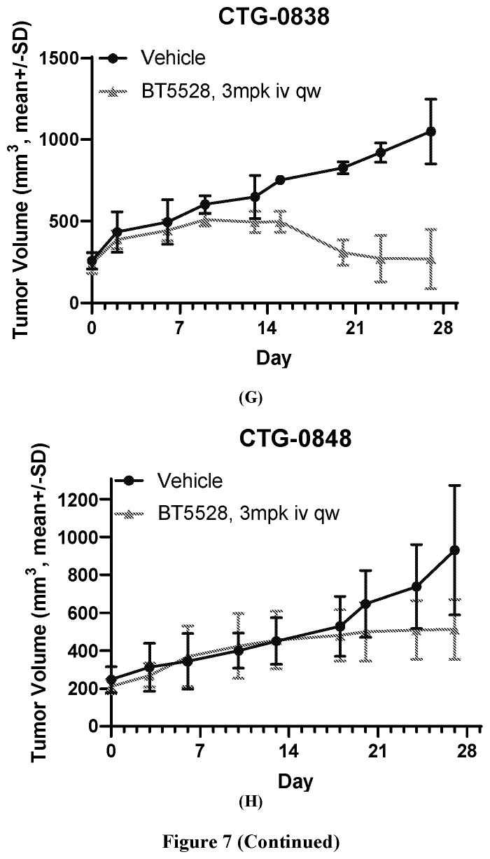

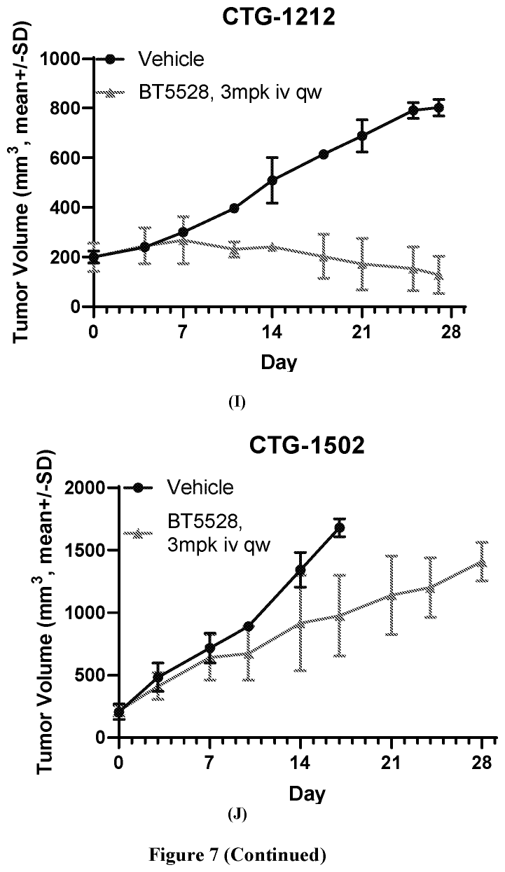

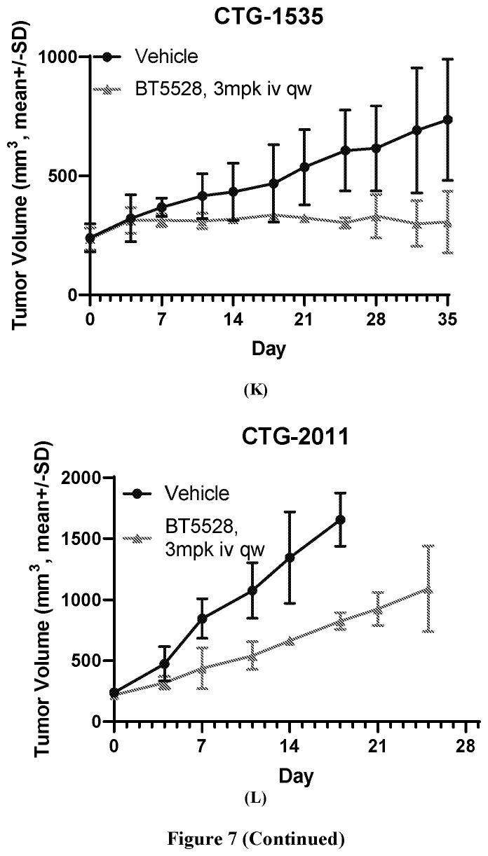

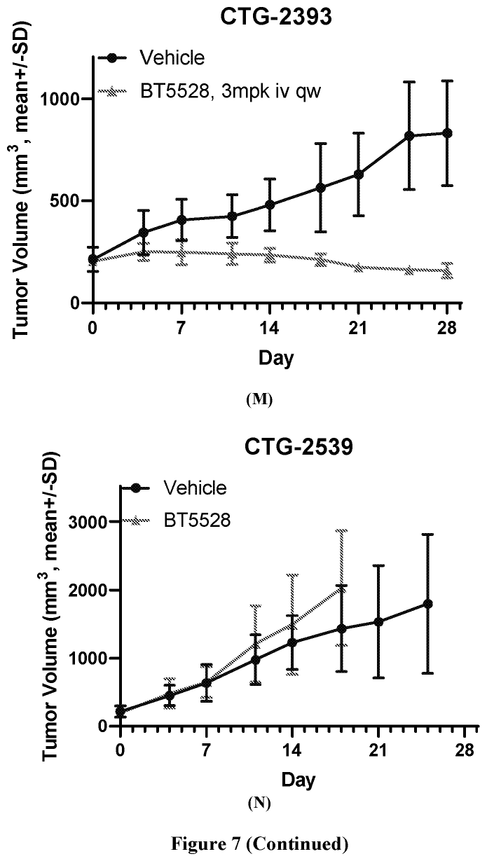

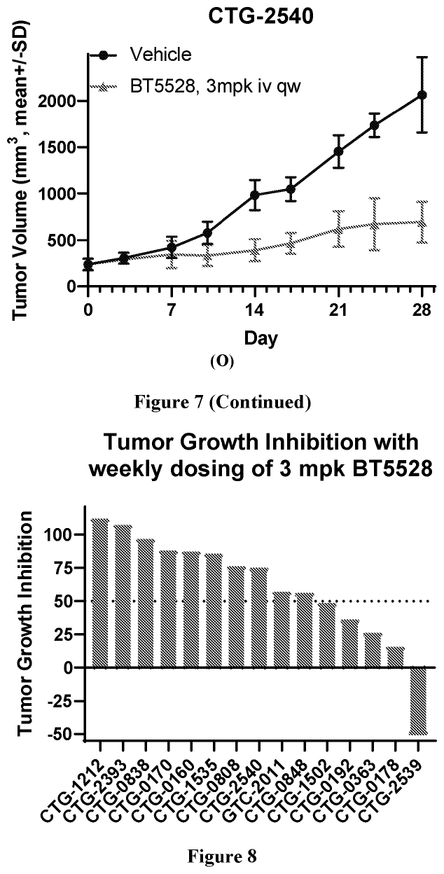

The doubling time of the tumor in the Vehicle Control was 8.0 days (FIG. 7A).

Animals were dosed according to the schedule on Table 7-1. The Vehicle Control group reached endpoint of average tumor volume >1500 mm³ on Day 21 and was removed from the study. The study was terminated on Day 28. Therefore, the tumor volumes on Day 21 were used for analysis of anti-tumor activity. The mean tumor volumes over the duration of the study are shown in FIG. 7A.

Intravenous treatment (n=2) with BT5528 at 3 mg/kg Q7Dx4 (MTV=400±187 mm³, TGI=87%, adjusted p=0.1021) decreased tumor volume compared to the Vehicle Control (MTV=1504±514 mm³) on Day 21 (Table 7-2 and FIG. 7A). There was no PR, CR or TFS in any group.

TABLE 7-2
Anti-Tumor Activity for NSCLC Model CTG-0160

| | | | | Day 21 Tumor Volume | | | |
|---|---|---|---|---|---|---|---|
| | | | IV Dose | Mean TV ± | p-Value vs. | | Day 0-28 |
| Group | Treatment | n | (mg/kg) | SEM | Vehicle* | % TGI | # PR/CR/TFS |
| 1 | Vehicle | 2 | 0, Q7Dx4 | 1504 ± 514 | — | — | 0/0/0 |
| 2 | BT5528 | 2 | 3, Q7Dx4 | 400 ± 187 | 0.1021 | 87 | 0/0/0 |

*One-way ANOVA followed by Dunnett's multiple comparisons test.

The Vehicle Control and treatment groups had no mean body weight loss. There was no death or moribund animal in any group. All treatment was tolerated in this study.

### 4.2 Efficacy Study in Non-Small Cell Lung Cancer Model CTG-0170

The doubling time of the tumor in the Vehicle Control was 4.5 days (FIG. 7B).

Animals were dosed according to the schedule on Table 7-1. The Vehicle Control group reached endpoint of average tumor volume >1500 mm³ on Day 14 and was removed from the study. The study was terminated on Day 28. However, one animal in the BT5528 group was found dead on Day 14. Therefore, the tumor volumes on Day 11 were used for analysis of anti-tumor activity in order to include all animals. The mean tumor volumes over the duration of the study are shown in FIG. 7B.

Intravenous treatment (n=2) with BT5528 at 3 mg/kg Q7Dx4 (MTV=268±15 mm³, TGI=94%, adjusted p=0.1283) decreased tumor volume compared to the Vehicle Control (MTV=1430±414 mm³) on Day 11 (Table 7-3 and FIG. 7B). There was no PR, CR or TFS in any group.

TABLE 7-3
Anti-Tumor Activity for NSCLC Model CTG-0170

| | | | | Day 11 Tumor Volume | | | |
|---|---|---|---|---|---|---|---|
| | | | IV Dose | Mean TV ± | p-Value vs. | | Day 0-28 |
| Group | Treatment | n | (mg/kg) | SEM | Vehicle* | % TGI | # PR/CR/TFS |
| 1 | Vehicle | 2 | 0, Q7Dx3 | 1430 ± 414 | — | — | 0/0/0 |
| 2 | BT5528 | 2 | 3, Q7Dx4 | 268 ± 15 | 0.1283 | 94 | 0/0/0 |

*One-way ANOVA followed by Dunnett's multiple comparisons test.

The Vehicle Control and treatment group had no mean body weight loss. One animal in the BT5528 was found dead on Day 14 with prior body weight loss of 24.8% and clinical observation of being thin. The cause of death was unknown. The other animal in the same group had no body weight loss and tolerated the BT5528 well.

### 4.3 Efficacy Study in Non-Small Cell Lung Cancer Model CTG-0178

The doubling time of the tumor in the Vehicle Control was 11.7 days (FIG. 7C).

Animals were dosed according to the schedule on Table 7-1. The study was terminated on Day 28. The mean tumor volumes over the duration of the study are shown in FIG. 7C.

Intravenous treatment (n=2) with BT5528 at 3 mg/kg Q7Dx4 (MTV=1145±271 mm³, TGI=16%, adjusted p=0.8566) decreased tumor volume compared to the Vehicle Control (MTV=1369±352 mm³) on Day 28 (Table 7-4 and FIG. 7C). There was no PR, CR or TFS in any group.

TABLE 7-4
Anti-Tumor Activity for NSCLC Mode CTG-0178

| | | | | Day 28 Tumor Volume | | | |
|---|---|---|---|---|---|---|---|
| | | | IV Dose | Mean TV ± | p-Value vs. | | Day 0-28 |
| Group | Treatment | n | (mg/kg) | SEM | Vehicle* | % TGI | # PR/CR/TFS |
| 1 | Vehicle | 2 | 0, Q7Dx4 | 1369 ± 352 | — | — | 0/0/0 |
| 2 | BT5528 | 2 | 3, Q7Dx4 | 1145 ± 271 | 0.8566 | 16 | 0/0/0 |

*One-way ANOVA followed by Dunnett's multiple comparisons test.

The Vehicle Control and BT5528 group had minor mean body weight losses on Day 18. There was no death or moribund animal in any group. All treatments were tolerated in this study.

### 4.4 Efficacy Study in Non-Small Cell Lung Cancer Model CTG-0192

The doubling time of the tumor in the Vehicle Control was 15.8 days (FIG. 7D).

Animals were dosed according to the schedule on Table 7-1. The study was terminated on Day 34. The mean tumor volumes over the duration of the study are shown in FIG. 7D.

Intravenous treatment (n=2) with BT5528 at 3 mg/kg Q7Dx5 (MTV=740±386 mm³, TGI=36%, adjusted p=0.7921) decreased tumor volume compared to the Vehicle Control (MTV=1020±259 mm³) on Day 34 (Table 7-5 and FIG. 7D). There was no PR, CR or TFS in any group.

TABLE 7-5
Anti-Tumor Activity for NSCLC Model CTG-0192

| | | | | Day 34 Tumor Volume | | | |
|---|---|---|---|---|---|---|---|
| | | | IV Dose | Mean TV ± | p-Value vs. | | Day 0-34 |
| Group | Treatment | n | (mg/kg) | SEM | Vehicle* | % TGI | # PR/CR/TFS |
| 1 | Vehicle | 2 | 0, Q7Dx5 | 1020 ± 259 | — | — | 0/0/0 |
| 2 | BT5528 | 2 | 3, Q7Dx5 | 740 ± 386 | 0.7921 | 36 | 0/0/0 |

*One-way ANOVA followed by Dunnett's multiple comparisons test.

The BT5528 group had minor mean body weight losses with maximum loss of 2.3% on Day 7. There was no death or moribund animal in any group. All treatments were tolerated in this study.

### 4.5 Efficacy Study in Non-Small Cell Lung Cancer Model CTG-0363

The doubling time of the tumor in the Vehicle Control was 9.6 days (FIG. 7E).

Animals were dosed according to the schedule on Table 7-1. The study was terminated on Day 28. The mean tumor volumes over the duration of the study are shown in FIG. 7E.

Intravenous treatment (n=2) with BT5528 at 3 mg/kg Q7Dx4 (MTV=1609±18 mm³, TGI=26%, adjusted p=0.2618) decreased tumor volume compared to the Vehicle Control on Day 28 (MTV=2170±53 mm³) on Day 28 (Table 7-6). There was no PR, CR or TFS in any group.

TABLE 7-6
Anti-Tumor Activity for NSCLC Model CTG-0363

| | | | | Day 28 Tumor Volume | | | |
|---|---|---|---|---|---|---|---|
| | | | IV Dose | Mean TV ± | p-Value vs. | | Day 0-28 |
| Group | Treatment | n | (mg/kg) | SEM | Vehicle* | % TGI | # PR/CR/TFS |
| 1 | Vehicle | 2 | 0, Q7Dx4 | 2170 ± 53 | — | — | 0/0/0 |
| 2 | BT5528 | 2 | 3, Q7Dx4 | 1609 ± 18 | 0.2618 | 26 | 0/0/0 |

*One-way ANOVA followed by Dunnett's multiple comparisons test.

The BT5528 group had no mean body weight loss. There was no death or moribund animal in any group. All treatments were tolerated in this study.

### 4.6 Efficacy Study in Non-Small Cell Lung Cancer Model CTG-0808

The doubling time of the tumor in the Vehicle Control was 5.0 days (FIG. 7F).

Animals were dosed according to the schedule on Table 7-1. The Vehicle Control reached endpoint on Day 14 and the study was terminated on Day 28. The mean tumor volumes over the duration of the study are shown in FIG. 7F.

Intravenous treatment (n=2) with BT5528 at 3 mg/kg Q7Dx4 (MTV=541±4 mm³, TGI=76%, adjusted p=0.0877) decreased tumor volume compared to the Vehicle Control on Day 14 (MTV=1616±501 mm³) on Day 14 (Table 7-7). There was no PR, CR or TFS in any group.

TABLE 7-7
Anti-Tumor Activity for NSCLC Model CTG-0808

| | | | | Day 14 Tumor Volume | | | |
|---|---|---|---|---|---|---|---|
| | | | IV Dose | Mean TV ± | p-Value vs. | | Day 0-28 |
| Group | Treatment | n | (mg/kg) | SEM | Vehicle* | % TGI | # PR/CR/TFS |
| 1 | Vehicle | 2 | 0, Q7Dx3 | 1616 ± 501 | — | — | 0/0/0 |
| 2 | BT5528 | 2 | 3, Q7Dx4 | 541 ± 4 | 0.0877 | 76 | 0/0/0 |

*One-way ANOVA followed by Dunnett's multiple comparisons test.

The Vehicle Control and treatment groups had no mean body weight loss. There was no death or moribund animal in any group. All treatments were tolerated in this study.

### 4.7 Efficacy Study in Non-Small Cell Lung Cancer Model CTG-0838

The doubling time of the tumor in the Vehicle Control was 13.3 days (FIG. 7G).

Animals were dosed according to the schedule on Table 7-1. The study was terminated on Day 27. The mean tumor volumes over the duration of the study are shown in FIG. 7G.

Intravenous treatment (n=2) with BT5528 at 3 mg/kg Q7Dx4 (MTV=269±128 mm³, TGI=97%, adjusted p=0.0153) decreased tumor volume compared to the Vehicle Control on Day 27 (MTV=1049±142 mm³) on Day 27 (Table 7-8). There was no PR, CR or TFS in any group.

TABLE 7-8
Anti-Tumor Activity for NSCLC Mode CTG-0838

| | | | | Day 27 Tumor Volume | | | |
|---|---|---|---|---|---|---|---|
| | | | IV Dose | Mean TV ± | p-Value vs. | | Day 0-27 |
| Group | Treatment | n | (mg/kg) | SEM | Vehicle* | % TGI | # PR/CR/TFS |
| 1 | Vehicle | 2 | 0, Q7Dx4 | 1049 ± 142 | — | — | 0/0/0 |
| 2 | BT5528 | 2 | 3, Q7Dx4 | 269 ± 128 | 0.0153 | 97 | 0/0/0 |

*One-way ANOVA followed by Dunnett's multiple comparisons test.

The Vehicle Control and the BT5528 group had no mean body weight loss. There was no death or moribund animal in any group. All treatments were tolerated in this study.

### 4.8 Efficacy Study in Non-Small Cell Lung Cancer Model CTG-0848

The doubling time of the tumor in the Vehicle Control was 14.1 days (FIG. 7H).

Animals were dosed according to the schedule on Table 7-1. The study was terminated on Day 27. The mean tumor volumes over the duration of the study are shown in FIG. 7H.

Treatment with BT5528 at 3 mg/kg Q7Dx4 (MTV=512±113 mm³, TGI=56%, adjusted p=0.1967) decreased tumor volume compared to the Vehicle Control on Day 27 (Table 7-9 and FIG. 7H). There was no PR, CR or TFS in any group.

TABLE 7-9
Anti-Tumor Activity for NSCLC Model CTG-0848

| | | | | Day 27 Tumor Volume | | | |
|---|---|---|---|---|---|---|---|
| | | | IV Dose | Mean TV ± | p-Value vs. | | Day 0-27 |
| Group | Treatment | n | (mg/kg) | SEM | Vehicle* | % TGI | # PR/CR/TFS |
| 1 | Vehicle | 2 | 0, Q7Dx4 | 931 ± 242 | — | — | 0/0/0 |
| 2 | BT5528 | 2 | 3, Q7Dx4 | 512 ± 113 | 0.1967 | 56 | 0/0/0 |

*One-way ANOVA followed by Dunnett's multiple comparisons test.

There was no death or moribund animal in any group. All treatments were tolerated in this study.

### 4.9 Efficacy Study in Non-Small Cell Lung Cancer Model CTG-1212

The doubling time of the tumor in the Vehicle Control was 13.5 days (FIG. 7I).

Animals were dosed according to the schedule on Table 7-1. The study was terminated on Day 27. The mean tumor volumes over the duration of the study are shown in FIG. 7I.

Intravenous treatment (n=2) with BT5528 at 3 mg/kg Q7Dx4 (MTV=128±53 mm³, TGI=112%, adjusted p=0.0020) decreased tumor volume compared to the Vehicle Control on Day 27 (MTV=802±24 mm³) on Day 27 (Table 7-10). There was no PR, CR or TFS in any group.

TABLE 7-10
Anti-Tumor Activity for NSCLC Model CTG-1212

| | | | | Day 27 Tumor Volume | | | |
|---|---|---|---|---|---|---|---|
| | | | IV Dose | Mean TV ± | p-Value vs. | | Day 0-27 |
| Group | Treatment | n | (mg/kg) | SEM | Vehicle* | % TGI | # PR/CR/TFS |
| 1 | Vehicle | 2 | 0, Q7Dx4 | 802 ± 24 | — | — | 0/0/0 |
| 2 | BT5528 | 2 | 3, Q7Dx4 | 128 ± 53 | 0.0020 | 112 | 0/0/0 |

*One-way ANOVA followed by Dunnett's multiple comparisons test.

The Vehicle Control and BT5528 group had minor mean body weight losses. There was no death or moribund animal in any group. All treatments were tolerated in this study.

### 4.10 Efficacy Study in Non-Small Cell Lung Cancer Model CTG-1502

The doubling time of the tumor in the Vehicle Control was 5.6 days (FIG. 7J).

Animals were dosed according to the schedule on Table 7-1. The Vehicle Control reached endpoint on Day 17 and the study was terminated on Day 28. The mean tumor volumes over the duration of the study are shown in FIG. 7J.

Intravenous treatment (n=2) with BT5528 at 3 mg/kg Q7Dx4 (MTV=976±229 mm³, TGI=48%, adjusted p=0.0484) decreased tumor volume compared to the Vehicle Control on Day 17 (MTV=1679±51 mm³) on Day 17 (Table 7-11). There was no PR, CR or TFS in any group.

TABLE 7-11
Anti-Tumor Activity for NSCLC Model CTG-1502

| | | | | Day 17 Tumor Volume | | | |
|---|---|---|---|---|---|---|---|
| | | | IV Dose | Mean TV ± | p-Value vs. | | Day 0-28 |
| Group | Treatment | n | (mg/kg) | SEM | Vehicle* | % TGI | # PR/CR/TFS |
| 1 | Vehicle | 2 | 0, Q7Dx3 | 1679 ± 51 | — | — | 0/0/0 |
| 2 | BT5528 | 2 | 3, Q7Dx4 | 976 ± 229 | 0.0484 | 48 | 0/0/0 |

*One-way ANOVA followed by Dunnett's multiple comparisons test.

The Vehicle Control and treatment groups had no mean body weight loss. There was no death or moribund animal in any group. All treatments were tolerated in this study.

### 4.11 Efficacy Study in Non-Small Cell Lung Cancer Model CTG-1535

The doubling time of the tumor in the Vehicle Control was 21.6 days (FIG. 7K).

Animals were dosed according to the schedule on Table 7-1. The study was terminated on Day 35. The mean tumor volumes over the duration of the study are shown in FIG. 7K.

Intravenous treatment (n=2) with BT5528 at 3 mg/kg Q7Dx5 (MTV=307±91 mm³, TGI=85%, adjusted p=0.2136) decreased tumor volume compared to the Vehicle Control on Day 35 (MTV=736±181 mm³) on Day 35 (Table 7-12). There was no PR, CR or TFS in any group.

TABLE 7-12
Anti-Tumor Activity for NSCLC Model CTG-1535

| | | | | Day 35 Tumor Volume | | | |
|---|---|---|---|---|---|---|---|
| | | | IV Dose | Mean TV ± | p-Value vs. | | Day 0-35 |
| Group | Treatment | n | (mg/kg) | SEM | Vehicle* | % TGI | # PR/CR/TFS |
| 1 | Vehicle | 2 | 0, Q7Dx5 | 736 ± 181 | — | — | 0/0/0 |
| 2 | BT5528 | 2 | 3, Q7Dx5 | 307 ± 91 | 0.2136 | 85 | 0/0/0 |

*One-way ANOVA followed by Dunnett's multiple comparisons test.

The Vehicle Control and treatment groups had no mean body weight loss. There was no death or moribund animal in any group. All treatments were tolerated in this study.

### 4.12 Efficacy Study in Non-Small Cell Lung Cancer Model CTG-2011

The doubling time of the tumor in the Vehicle Control was 6.5 days (FIG. 7L).

Animals were dosed according to the schedule on Table 7-1. The Vehicle Control reached endpoint on Day 18 and the study was terminated on Day 28. The mean tumor volumes over the duration of the study are shown in FIG. 7L.

Intravenous treatment (n=2) with BT5528 at 3 mg/kg Q7Dx4 (MTV=826±48 mm³, TGI=57%, adjusted p=0.0852) decreased tumor volume compared to the Vehicle Control on Day 18 (MTV=1655±155 mm³) on Day 18 (Table 7-13). There was no PR, CR or TFS in any group.

TABLE 7-13
Anti-Tumor Activity for NSCLC Model CTG-2011

| | | | | Day 18 Tumor Volume | | | |
|---|---|---|---|---|---|---|---|
| | | | IV Dose | Mean TV ± | p-Value vs. | | Day 0-28 |
| Group | Treatment | n | (mg/kg) | SEM | Vehicle* | % TGI | # PR/CR/TFS |
| 1 | Vehicle | 2 | 0, Q7Dx3 | 1655 ± 155 | — | — | 0/0/0 |
| 2 | BT5528 | 2 | 3, Q7Dx4 | 826 ± 48 | 0.0852 | 57 | 0 /0/0 |

*One-way ANOVA followed by Dunnett's multiple comparisons test.

The Vehicle Control and treatment groups had no mean body weight loss. All treatments were tolerated in this study.

### 4.13 Efficacy Study in Non-Small Cell Lung Cancer Model CTG-2393

The doubling time of the tumor in the Vehicle Control was 14.3 days (FIG. 7M).

Animals were dosed according to the schedule on Table 7-1. The study was terminated on Day 28. The mean tumor volumes over the duration of the study are shown in FIG. 7M.

Intravenous treatment (n=2) with BT5528 at 3 mg/kg Q7Dx4 (MTV=158±25 mm³, TGI=107%, adjusted p=0.0185) decreased tumor volume compared to the Vehicle Control on Day 28 (MTV=831±182 mm³) on Day 28 (Table 7-14). There was no PR, CR or TFS in any group.

TABLE 7-14
Anti-Tumor Activity for NSCLC Model CTG-2393

| | | | | Day 28 Tumor Volume | | | |
|---|---|---|---|---|---|---|---|
| | | | IV Dose | Mean TV ± | p-Value vs. | | Day 0-28 |
| Group | Treatment | n | (mg/kg) | SEM | Vehicle* | % TGI | # PR/CR/TFS |
| 1 | Vehicle | 2 | 0, Q7Dx4 | 831 ± 182 | — | — | 0/0/0 |
| 2 | BT5528 | 2 | 3, Q7Dx4 | 158 ± 25 | 0.0185 | 107 | 0/0/0 |

*One-way ANOVA followed by Dunnett's multiple comparisons test.

There was no death or moribund animal in any group. All treatments were tolerated in this study.

### 4.14 Efficacy Study in Non-Small Cell Lung Cancer Model CTG-2539

The doubling time of the tumor in the Vehicle Control was 8.3 days (FIG. 7N).

Animals were dosed according to the schedule on Table 7-1. The Vehicle Control reached endpoint on Day 21 and the BT5528 group reached endpoint on Day 18. The study was terminated on Day 25. The mean tumor volumes over the duration of the study are shown in FIG. 7N.

Intravenous treatment (n=2) with BT5528 at 3 mg/kg Q7Dx3 (MTV=2029±594 mm³, TGI=−51%, adjusted p=0.7171) did not decrease tumor volume compared to the Vehicle Control on Day 18 (MTV=1433±448 mm³) on Day 18 (Table 7-15). There was no PR, CR or TFS in any group.

The Vehicle Control and treatment groups had no mean body weight loss. There was no death or moribund animal in any group. All treatments were tolerated in this study.

TABLE 7-15
Anti-Tumor Activity for NSCLC Model CTG-2539

| | | | | Day 18 Tumor Volume | | | |
|---|---|---|---|---|---|---|---|
| | | | IV Dose | Mean TV ± | p-Value vs. | | Day 0-25 |
| Group | Treatment | n | (mg/kg) | SEM | Vehicle* | % TGI | # PR/CR/TFS |
| 1 | Vehicle | 2 | 0, Q7Dx4 | 1433 ± 448 | — | — | 0/0/0 |
| 2 | BT5528 | 2 | 3, Q7Dx3 | 2029 ± 594 | 0.7171 | −51 | 0/0/0 |

*One-way ANOVA followed by Dunnett's multiple comparisons test.

### 4.15 Efficacy Study in Non-Small Cell Lung Cancer Model CTG-2540

The doubling time of the tumor in the Vehicle Control was 9.0 days (FIG. 7O).

Animals were dosed according to the schedule on Table 7-1. The study was terminated on Day 28. The mean tumor volumes over the duration of the study are shown in FIG. 7O.

Intravenous treatment (n=2) with BT5528 at 3 mg/kg Q7Dx4 (MTV=692±156 mm³, TGI=75%, adjusted p=0.0324) decreased tumor volume compared to the Vehicle Control on Day 28 (MTV=2065±289 mm³) on Day 28 (Table 7-16). There was no PR, CR or TFS in any group.

TABLE 7-16
Anti-Tumor Activity for NSCLC Model CTG-2540

| | | | | Day 28 Tumor Volume | | | |
|---|---|---|---|---|---|---|---|
| | | | IV Dose | Mean TV ± | p-Value vs. | | Day 0-28 |
| Group | Treatment | n | (mg/kg) | SEM | Vehicle* | % TGI | # PR/CR/TFS |
| 1 | Vehicle | 2 | 0, Q7Dx4 | 2065 ± 289 | — | — | 0/0/0 |
| 2 | BT5528 | 2 | 3, Q7Dx4 | 692 ± 156 | 0.0324 | 75 | 0/0/0 |

*One-way ANOVA followed by Dunnett's multiple comparisons test.

The Vehicle Control and the BT5528 group had no mean body weight loss. There was no death or moribund animal in any group. All treatments were tolerated in this study.

1. A method of preventing or treating a disease, disorder, or condition characterised by overexpression of EphA2 in a patient, comprising administering to the patient Bicycle toxin conjugate BT5528, or a pharmaceutically acceptable salt thereof, or a pharmaceutical composition thereof.

2. The method of claim 1, wherein Bicycle toxin conjugate BT5528, or a pharmaceutically acceptable salt thereof, or a pharmaceutical composition thereof, is administered to a patient to provide a system exposure of BT5528 and/or MMAE for about 2 hours or less.

3. The method of claim 2, wherein the system exposure of BT5528 is measured by the time when the concentration of BT5528 in plasma is about 2% or more of the maximum concentration of BT5528 in plasma.

4. The method of claim 2 or 3, wherein the system exposure of BT5528 is measured by the time when the concentration of BT5528 in plasma is about 15 pmol/gram or more.

5. The method of claim 2, wherein the system exposure of MMAE is measured by the time when the concentration of MMAE in plasma is about 10% or more of the maximum concentration of MMAE in plasma.

6. The method of claim 2 or 5, wherein the system exposure of MMAE is measured by the time when the concentration of MMAE in plasma is about 2 pmol/gram or more.

7. The method of any one of the preceding claims, wherein Bicycle toxin conjugate BT5528, or a pharmaceutically acceptable salt thereof, or a pharmaceutical composition thereof, is administered to a patient to provide a tumor MMAE concentration of about 50 pmol/gram or more.

8. The method of any one of the preceding claims, wherein Bicycle toxin conjugate BT5528, or a pharmaceutically acceptable salt thereof, or a pharmaceutical composition thereof, is administered to a patient to induce mitotic arrest in tumor within about 24 hours.

9. The method of claim 8, wherein mitotic arrest in tumor is induced when there is about 15% or more pHH3+ nuclei in tumor.

10. The method of any one of the preceding claims, wherein Bicycle toxin conjugate BT5528, or a pharmaceutically acceptable salt thereof, or a pharmaceutical composition thereof, is administered to a patient to induce measurable tumor regression by day 4 post dosing.

11. The method of any one of the preceding claims, wherein Bicycle toxin conjugate BT5528, or a pharmaceutically acceptable salt thereof, or a pharmaceutical composition thereof, is administered to a patient at about 0.3 mg/m² to about 9 mg/m² each dose.

12. The method of any one of the preceding claims, wherein Bicycle toxin conjugate BT5528, or a pharmaceutically acceptable salt thereof, or a pharmaceutical composition thereof, is administered to a patient by an intravenous bolus injection.

13. The method of any one of claims 1-11, wherein Bicycle toxin conjugate BT5528, or a pharmaceutically acceptable salt thereof, or a pharmaceutical composition thereof, is administered to a patient by an intravenous infusion.

14. The method of claim 13, wherein the intravenous infusion is an about 1 hour infusion.

15. The method of any one of claims 1-11, wherein Bicycle toxin conjugate BT5528, or a pharmaceutically acceptable salt thereof, or a pharmaceutical composition thereof, is administered to a patient by a subcutaneous infusion.

16. The method of claim 15, wherein the subcutaneous infusion is an about 24 hour infusion.

17. The method of any one of the preceding claims, wherein Bicycle toxin conjugate BT5528, or a pharmaceutically acceptable salt thereof, or a pharmaceutical composition thereof, is administered to a patient two or more times, with at least 48 hours in between two consecutive administrations.

18. The method of any one of the preceding claims, wherein Bicycle toxin conjugate BT5528, or a pharmaceutically acceptable salt thereof, or a pharmaceutical composition thereof, is administered to a patient two or more times, with about one week in between two consecutive administrations.

19. A Bicycle toxin conjugate, which is BT5528 as shown herein, or a pharmaceutically acceptable salt thereof.

20. A pharmaceutical composition comprising Bicycle toxin conjugate BT5528, or a pharmaceutically acceptable salt thereof, and a pharmaceutically acceptable carrier, adjuvant, or vehicle.

21. A Bicycle toxin conjugate, which is BCY10188 as shown herein, or a pharmaceutically acceptable salt thereof.

22. A pharmaceutical composition comprising Bicycle toxin conjugate BCY10188, or a pharmaceutically acceptable salt thereof, and a pharmaceutically acceptable carrier, adjuvant, or vehicle.

23. A method of preventing or treating a disease, disorder, or condition characterised by overexpression of EphA2 in a patient, comprising administering to the patient Bicycle toxin conjugate BCY10188, or a pharmaceutically acceptable salt thereof, or a pharmaceutical composition thereof.

24. The method of claim 23, wherein Bicycle toxin conjugate BCY10188, or a pharmaceutically acceptable salt thereof, or a pharmaceutical composition thereof, is administered to a patient at about 0.3 mg/m² to about 9 mg/m² each dose.

25. The method of claim 23 or 24, wherein Bicycle toxin conjugate BCY10188, or a pharmaceutically acceptable salt thereof, or a pharmaceutical composition thereof, is administered to a patient by an intravenous bolus injection.

26. The method of any one of claims 1-18 and 23-25, comprising administering one or more other therapeutic agent.

27. The method of any one of claims 1-18 and 23-26, wherein the disease, disorder, or condition characterised by overexpression of EphA2 is a cancer.

28. The method of claim 27, wherein the cancer is selected from prostate cancer, lung cancer (such as non-small cell lung carcinomas (NSCLC)), breast cancer (such as triple negative breast cancer), gastric cancer, ovarian cancer, oesophageal cancer, multiple myeloma, pancreatic cancer, and fibrosarcoma.


SEQUENCE LISTING (2 sequences)

SEQ ID NO: 1 - protein, 20 aa, Artificial
  Synthetic peptide
  Xaa (1)..(1): Xaa is beta-Ala
  Xaa (2)..(2): Xaa is Sar10
  Xaa (4)..(4): Xaa is HArg
  Xaa (7)..(7): Xaa is HyP
  Xaa (17)..(17): Xaa is D-Asp
  Xaa (19)..(19): Xaa is HArg
XXAXDCXLVNPLCLHPXWXC

SEQ ID NO: 2 - protein, 15 aa, Artificial
  Synthetic peptide
  misc_feature (2)..(7): Xaa can be any naturally occurring amino acid
  misc_feature (9)..(14): Xaa can be any naturally occurring amino acid
CXXXXXXCXXXXXXC